SEQUENCE LISTING (40 sequences)

SEQ ID NO: 1 - protein, 379 aa, Artificial Sequence
  Polypeptide sequence of Amatilin
MKYLLPTAAAGLLLLAAQPAMAMGRICRCICGRGICRCICGVPGVGVPGVGGATGSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPWALWKTMLKELGTMALHAGKAALGAAADTISQGTQVPGVGVPGVGKLAAALEHHHHHH

SEQ ID NO: 2 - DNA, 1140 bp, Artificial Sequence
  Coding sequence of Amatilin
atgaaatacctgctgccgaccgctgctgctggtctgctgctcctcgctgcccagccggcgatggccatggggcgtatttgccgttgcatttgcggccgtggcatttgccgctgcatctgtggcgtgccgggtgttggtgttccgggtgtgggtggtgcgaccggatccgatgtgaactttgatctgagcaccgcgaccgcgaaaacctataccaaattcatcgaagattttcgtgcgaccctgccgtttagccataaagtgtatgatatcccgctgctgtatagcaccattagcgatagccgtcgttttattctgctggatctgaccagctatgcgtatgaaaccattagcgtggcgattgatgtgaccaacgtgtatgtggtggcgtatcgtacccgtgatgtgagctactttttcaaagaaagcccgccggaagcgtacaacattctgtttaaaggcacccgtaaaattaccctgccgtataccggcaactatgaaaacctgcagaccgcggcgcataaaattcgtgaaaacatcgatctgggcctgccggccctgagcagcgcgattaccaccctgttttattataacgcgcagagcgcgccgagcgcgctgctggtgctgattcagaccaccgcggaagcggcgcgttttaaatatattgaacgccacgtggcgaaatatgtggcgaccaactttaaaccgaacctggccattattagcctggaaaaccagtggagcgccctgagcaaacaaatttttctggcccagaaccagggcggcaaatttcgtaatccggtggatctgattaaaccgaccggcgaacgttttcaggtgaccaatgtggatagcgatgtggtgaaaggcaacattaaactgctgctgaacagccgtgcgagcaccgcggatgaaaactttattaccaccatgaccctgctgggcgaaagcgtggtggaattcccgtgggcgctgtggaaaaccatgctgaaagaactgggcaccatggcgctgcatgcgggtaaagcggcgctgggtgcggcagcggataccattagccagggcacccaggttccgggcgtgggcgttccgggcgttggtaagcttgcggccgcactcgagcaccaccaccaccaccactga

SEQ ID NO: 3 - protein, 5 aa, Artificial Sequence
  A polypeptide of the present invention
VPXVG

SEQ ID NO: 4 - protein, 18 aa, Artificial Sequence
  A polypeptide of the present invention
GRICRCICGRGICRCICG

SEQ ID NO: 5 - protein, 268 aa, Artificial Sequence
  A polypeptide of the present invention
GSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPW

SEQ ID NO: 6 - protein, 34 aa, Artificial Sequence
  A polypeptide of the present invention
ALWKTMLKELGTMALHAGKAALGAAADTISQGTQ

SEQ ID NO: 7 - protein, 18 aa, Artificial Sequence
  A polypeptide sequence of naturally occurring and synthetic theta Defensin proteins
GFCRCLCRRGVCRCICTR

SEQ ID NO: 8 - protein, 18 aa, Artificial Sequence
  A polypeptide sequence of naturally occurring and synthetic theta Defensin proteins
RCLCRRGVCRCLCRRGVC

SEQ ID NO: 9 - protein, 18 aa, Artificial Sequence
  A polypeptide sequence of naturally occurring and synthetic theta Defensin proteins
RCICTRGFCRCICTRGFC

SEQ ID NO: 10 - protein, 18 aa, Artificial Sequence
  A polypeptide sequence of naturally occurring and synthetic theta Defensin proteins
GICRCICGRGICRCICGR

SEQ ID NO: 11 - protein, 18 aa, Artificial Sequence
  A polypeptide sequence of naturally occurring and synthetic theta Defensin proteins
GICRCICGRGICRCICGR

SEQ ID NO: 12 - protein, 18 aa, Artificial Sequence
  A polypeptide sequence of naturally occurring and synthetic theta Defensin proteins
RICRCICGRRICRCICGR

SEQ ID NO: 13 - protein, 18 aa, Artificial Sequence
  A polypeptide sequence of naturally occurring and synthetic theta Defensin proteins
GICRCICGRGICRCICGR

SEQ ID NO: 14 - protein, 18 aa, Artificial Sequence
  A polypeptide sequence of naturally occurring and synthetic theta Defensin proteins
GICRCICGKGICRCICGR

SEQ ID NO: 15 - protein, 18 aa, Artificial Sequence
  A polypeptide sequence of naturally occurring and synthetic theta Defensin proteins
GICRCYCGRGICRCICGR

SEQ ID NO: 16 - protein, 18 aa, Artificial Sequence
  A polypeptide sequence of naturally occurring and synthetic theta Defensin proteins
GICRCICGRGICRCYCGR

SEQ ID NO: 17 - protein, 18 aa, Artificial Sequence
  A polypeptide sequence of naturally occurring and synthetic theta Defensin proteins
GYCRCICGRGICRCICGR

SEQ ID NO: 18 - protein, 18 aa, Artificial Sequence
  A polypeptide sequence of naturally occurring and synthetic theta Defensin proteins
GICRCICGRGYCRCICGR

SEQ ID NO: 19 - protein, 18 aa, Artificial Sequence
  A polypeptide sequence of naturally occurring and synthetic theta Defensin proteins
GICYCICGRGICRCICGR

SEQ ID NO: 20 - protein, 18 aa, Artificial Sequence
  A polypeptide sequence of naturally occurring and synthetic theta Defensin proteins
GICICICGYGICRCICGR

SEQ ID NO: 21 - protein, 18 aa, Artificial Sequence
  A polypeptide sequence of naturally occurring and synthetic theta Defensin proteins
GICICICGRGICYCICGR

SEQ ID NO: 22 - protein, 18 aa, Artificial Sequence
  A polypeptide sequence of naturally occurring and synthetic theta Defensin proteins
GICICICGRGICYCICGR

SEQ ID NO: 23 - protein, 18 aa, Artificial Sequence
  A polypeptide sequence of naturally occurring and synthetic theta Defensin proteins
RGCICRCIGRGCICRCIG

SEQ ID NO: 24 - protein, 18 aa, Artificial Sequence
  A polypeptide sequence of naturally occurring and synthetic theta Defensin proteins
RGCICRCIGRGCICRCIG

SEQ ID NO: 25 - protein, 18 aa, Artificial Sequence
  A polypeptide sequence of naturally occurring and synthetic theta Defensin proteins
GICRCICGRGICRCICGR

SEQ ID NO: 26 - protein, 18 aa, Artificial Sequence
  A polypeptide sequence of naturally occurring and synthetic theta Defensin proteins
GICRCICGKGICRCYCGR

SEQ ID NO: 27 - protein, 4 aa, Artificial Sequence
  An example of fusion proteins according to the present invention
GGGS

SEQ ID NO: 28 - protein, 360 aa, Artificial Sequence
  An example of fusion proteins according to the present invention
SFGLCRLRRGFCAHGRCRFPSIPIGRCSRFVQCCRRVWVPGVGVPGVGGATGSDVNFDLSTATAKTYTKFIEDFRATLPFSHKVYDIPLLYSTISDSRRFILLNLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVNSCASRCKGHCRARRCGYYVSVLYRGRCYCKCLRCVPGVGVPGVG

SEQ ID NO: 29 - protein, 401 aa, Artificial Sequence
  An example of fusion proteins according to the present invention
LEKRKWKLFKKIEKVGQRVRDAVISAGPAVATVAQATALAKNVPGVGVPGVGGATGSDVSFRLSGATSKKKVYFISNLRKALPNEKKLYDIPLVRSSSGSKATAYTLNLANPSASQYSSFLDQIRNNVRDTSLIYGGTDVAVIGAPSTTDKFLRLNFQGPRGTVSLGLRRENLYVVAYLAMDNANVNRAYYFKNQITSAELTALFPEVVVANQKQLEYGEDYQAIEKNAKITTGDQSRKELGLGINLLITMIDGVNKKVRVVKDEARFLLIAIQMTAEAARFRYIQNLVTKNFPNKFDSENKVIQFQVSWSKISTAIFGDCKNGVFNKDYDFGFGKVRQAKDLQMGLLKYLGRPKSSSIEANSTDDTADVLVPGVGVPGVGKTCENLADTFRGPCFATSNC

SEQ ID NO: 30 - protein, 297 aa, Artificial Sequence
  An example of fusion proteins according to the present invention
MGRICRCICGRGICRCICGVPGVGVPGVGGSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPW

SEQ ID NO: 31 - protein, 297 aa, Artificial Sequence
  An example of fusion proteins according to the present invention
MGSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPWVPGVGVPGVGGRICRCICGRGICRCICG

SEQ ID NO: 32 - protein, 313 aa, Artificial Sequence
  An example of fusion proteins according to the present invention
MGSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPWVPGVGVPGVGALWKTMLKELGTMALHAGKAALGAAADTISQGTQ

SEQ ID NO: 33 - protein, 313 aa, Artificial Sequence
  An example of fusion proteins according to the present invention
MALWKTMLKELGTMALHAGKAALGAAADTISQGTQVPGVGVPGVGGSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPW

SEQ ID NO: 34 - protein, 328 aa, Artificial Sequence
  An example of fusion proteins according to the present invention
VPGVGVPGVGKWCFRVCYRGICYRRCRVPGVGVPGVGGATGSDVNFDLSTATAKTYTKFIEDFRATLPFSHKVYDIPLLYSTISDSRRFILLNLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVNVPGVGVPGVGHGVSGHGQHGVHG

SEQ ID NO: 35 - protein, 336 aa, Artificial Sequence
  An example of fusion proteins according to the present invention
VPGVGVPGVGFLPLLAGLAANFLPTIICFISYKCVPGVGVPGVGGATGSDVNFDLSTATAKTYTKFIEDFRATLPFSHKVYDIPLLYSTISDSRRFILLNLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVNVPGVGVPGVGKLAKLAKKLAKLAK

SEQ ID NO: 36 - protein, 325 aa, Artificial Sequence
  Polypeptide sequence of RetroGAD1
GICRCIGRGICRCICGRVPGVGVPGVGGATGSGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPKGLWSKIKEAAKAAGKAALNAVTGLVNQGDQPSVPGVGVPGVG

SEQ ID NO: 37 - DNA, 1185 bp, Artificial Sequence
  Coding sequence of Amatilin
gggcagtgagcggaaggcccatgaggccagttaattaagaggtaccgaattctcattcggtttgtgtagattgagaagaggtttctgtgctcacggtagatgtagattcccatccatcccaatcggtagatgttccagattcgttcagtgttgtagaagagtttgggtcccaggtgttggtgttccaggtgttggaggtgctactggttctgatgttaacttcgacttgtccactgctactgctaagacttacactaagttcatcgaggacttcagagctactttgccattctcccacaaggtttacgacatccctttgttgtactccactatctccgactccagaagattcatcttgttgaacttgacttcctacgcttacgagactatctccgttgctatcgacgttacaaacgtttacgttgttgcttacagaactagagatgtttcctacttcttcaaagagtccccaccagaggcttacaacatcttgttcaagggtactagaaagatcactttgccatacactggtaactacgagaacttgcagactgctgctcacaagatcagagagaacatcgacttgggtttgccagctttgtcctccgctatcactactttgttctactacaacgctcagtccgctccatccgctttgttggttttgatccagactactgctgaggctgctagattcaagtacatcgagagacacgttgctaagtacgttgctacaaacttcaagccaaacttggctatcatctccttggagaaccagtggtctgctttgtccaagcagatcttcttggctcaaaaccagggtggtaagttcagaaacccagtcgacttgatcaagccaaccggtgagagattccaggttactaatgttgactccgacgttgttaagggtaacatcaagttgttgttgaactccagagcttccactgctgacgagaacttcatcactactatgactttgttgggtgagtccgttgttaactcctgtgcttccagatgtaagggtcactgtagagctagaagatgtggttactacgtttccgttctgtacagaggtagatgttactgtaaatgtttgagatgtgtccccggtgttggagtccctggtgtcggtgcggccgcgagctcatggcgcgcctaggccttgacggccttccgccaattcgc

SEQ ID NO: 38 - DNA, 1084 bp, Artificial Sequence
  Coding sequence for RetroGAD1
cgaattggcggaaggccgtcaaggccacgtgtcttgtccaggtaccgaattcggaatctgtagatgcatctgcggtagaggtatctgcagatgtatttgtggaagagtcccaggtgttggtgttccaggtgttggaggtgctactggttctggtttggacactgtttcattctccactaagggtgctacttacatcacttacgttaactttttgaacgagttgagagttaagttgaagccagagggtaactcccacggtatccctttgttgagaaagaagtgtgacgacccaggtaagtgtttcgttttggttgctttgtccaacgacaacggtcagttggctgagattgctatcgacgttacttccgtttacgttgttggttaccaggttagaaacagatcctacttcttcaaggacgctccagacgctgcttacgaaggtttgttcaagaacactatcaagactagattgcacttcggtggttcctacccatctttggaaggtgagaaggcttacagagagactactgacttgggtatcgagccattgagaatcggtatcaagaagttggacgagaacgctatcgacaactacaagccaactgagatcgcttcctccttgttggttgttatccagatggtttccgaggctgctagattcactttcatcgagaaccagatcagaaacaacttccagcagagaatcagaccagctaacaacactatttccttggagaacaagtggggtaagttgtccttccagatcagaacatccggtgctaacggtatgttctctgaggctgttgagttggagagagctaacggtaagaagtactacgttactgctgttgaccaggttaagccaaagatcgctttgttgaagttcgttgacaaggacccaaagggtttgtggtccaagatcaaagaggctgctaaggctgctggtaaggctgctttgaatgctgttactggtttggttaaccagggtgaccaaccatctgtccctggtgttggagtccctggtgtcggtgcggccgcgagctctggagcacaagactggcctcatgggccttccgctcactgc

SEQ ID NO: 39 - DNA, 998 bp, Artificial Sequence
  Coding sequence of Tampal 1
ggatccgttccgggtgtgggtgttccgggtgttggtaaatggtgtttcgtgtttgttatcgcggtatttgttatcgtcgttgtcgtgtgccaggcgttggcgttccaggcgtgggtggtgcaaccggtagtgatgttaattttgatctgagcaccgcaaccgcaaaaacctataccaaatttatcgaagattttcgtgcaaccctgccgtttagccataaagtttatgatattccgctgctgtatagcaccattagcgatagccgtcgttttattctgctgaatctgaccagctatgcctatgaaaccattagcgttgcaattgatgtgaccaatgtttatgttgttgcatatcgtacccgtgatgtgagctattttttcaaagaaagccctccggaagcctataacattctgtttaaaggcacccgcaaaatcaccctgccgtataccggtaattatgaaaatctgcagaccgcagcacataaaattcgcgaaaatattgatctgggtctgcctgcactgagcagcgcaattaccaccctgttttattacaatgcacagagcgcaccgagcgcactgctggttctgattcagaccaccgcagaagcagcacgctttaaatacattgaacgtcatgttgccaaatacgtggccaccaactttaaaccgaatctggcaattattagcctggaaaatcagtggtcagcactgagcaaacaaatttttctggcacagaatcagggtggcaaatttcgtaatccggttgatctgattaaaccgaccggtgaacgttttcaggttaccaatgttgatagtgatgtggtgaaaggcaacattaaactgctgctgaatagccgtgcaagcaccgcagatgaaaactttattaccaccatgaccctgctgggtgaaagcgttgttaatgttcctggtgttggcgtgcctggtgttggtcatggtgttagcggtcatggtcagcatggtgttcatggttaaaagctt

SEQ ID NO: 40 - DNA, 1023 bp, Artificial Sequence
  Coding sequence of K5
ggatccgttccgggtgtgggtgttccgggtgttggctttctgggtgcactgtttaaagttgcaagcaaagttctgccgagcgttaaatgtgcaattaccaaaaaatgtgttcctggcgttggtgttccaggcgtgggtggtgcaaccggtagtgatgttaattttgatctgagcaccgcaaccgcaaaaacctataccaaatttatcgaagattttcgtgcaaccctgccgtttagccataaagtttatgatattccgctgctgtatagcaccattagcgatagccgtcgttttattctgctgaatctgaccagctatgcctatgaaaccattagcgttgcaattgatgtgaccaatgtttatgttgttgcatatcgtacccgtgatgtgagctattttttcaaagaaagccctccggaagcctataacattctgtttaaaggcacccgcaaaatcaccctgccgtataccggtaattatgaaaatctgcagaccgcagcacataaaattcgcgaaaatattgatctgggtctgcctgcactgagcagcgcaattaccaccctgttttattacaatgcacagagcgcaccgagcgcactgctggttctgattcagaccaccgcagaagcagcacgctttaaatacattgaacgtcatgttgccaaatacgtggccaccaactttaaaccgaatctggcaattattagcctggaaaatcagtggtcagcactgagcaaacaaatttttctggcacagaatcagggtggcaaatttcgtaatccggttgatctgattaaaccgaccggtgaacgttttcaggttaccaatgttgatagtgatgtggtgaaaggcaacattaaactgctgctgaatagccgtgcaagcaccgcagatgaaaactttattaccaccatgaccctgctgggtgaaagcgttgttaatgttccaggtgttggtgtgcctggtgtgggtaaactggcaaaactggccaaaaaactggctaagctggcgaaataaaagctt

(19) United States
(12) Patent Application Publication
(10) Pub. No.: US 2015/0202250 A1
(43) Pub. Date: Jul. 23, 2015

(54) ANTICANCER TREATMENT

(71) Applicant: BIOVALENCE SDN. BHD., Petaling Jaya (MY)

(72) Inventors: Shamala Devi K C Sekaran, Petaling Jaya (MY); Hussin A. Rothan, Kuala Lumpur (MY); Eng Huan Ung, Sabah (MY); Muhammad Sagaf Abu Bakar, Ag., Sabah (MY)

(21) Appl. No.: 14/408,780

(22) PCT Filed: Jun. 25, 2013
(86) PCT No.: PCT/MY2013/000115

§ 371 Date: Dec. 17, 2014

(30) Foreign Application Priority Data
Jun. 26, 2012 (MY) PI2012002925

(51) Int. Cl. A61K 38/16; A61K 38/02; A61K 38/10

(52) U.S. Cl. CPC A61K 38/16; A61K 38/10; A61K 38/02

(57) ABSTRACT

Use of fusion protein comprising at least one polypeptide B, comprising Type 1 Ribosome Inactivating Protein, and at least one C having anticancer properties in the manufacture of a medicament for treating cancer in a subject in need thereof.

### FIELD OF THE INVENTION

The present invention relates to the use of fusion compounds in cancer treatments.

### BACKGROUND TO THE INVENTION

There are over 200 different known cancers that afflict human beings. Cancer causes millions of deaths a year worldwide and rates are also rising as more people live to an older age and urbanization causes more stress. In is anticipated that one in eight people currently alive will eventually die of cancer. In 2008, an estimated 12.7 million people were diagnosed with cancer. In that same year, of the 56,888,000 deaths due to disease as recorded by the World Health Organization, 13.3% or 7,538,000 died of cancer, after cardiovascular disease (30.5%) and infections (15.3%). Of these cancer deaths, about 10% or 748,300 were due to liver cancer, the 5th most common cancer in males and 7th most common in females. At the moment, Sorafenib/Nexavar® is the only approved liver cancer drug on the market (Di Francesco, C (2007)).

Tumours are now recognized as comprising of a mosaic of genetically different and actively mutating cells rather than a single type. Thus combination drug therapies are being advocated to combat tumour cellular heterogenecity. The use of a sequential 1-2 or 1-2-3 therapy can also address the issue of immunogenicity in the case of biologic protein drugs as well as address the issue of drug resistance.

Also, 12-17.8% of all human cancers are caused by viral infections. For example, liver cancer is often associated with Hepatitis C and Flavivirus 7, and prostate cancer may be associated with HSV-2 etc.

Phosphatidylinositol 3-kinase (PI3K) signaling impacts cancer cell growth, survival, migration and metabolism. This pathway is activated by several different mechanisms in cancers and is a prime target for drug discovery especially with combination treatments, such as using mitogen-activated protein kinase kinase (MEK) with PI3K inhibitors to treat cancers with mutations in K-RAS1 and combining antibodies and PI3K in treatment of breast cancer with HER2 gene amplification. Combination therapy involving PI3K and the PARP inhibitor, Olaparib, for BRCA-mutant breast tumours have been shown to be effective in vivo models. PI3K mutations also induce increased cell migration independent of PTEN (phosphotase and tensin homolog deleted on chromosome 10) which directly opposes PI3K activity as a central negative regulator. Inhibition of PI3K also blocks the generation of leukemia-initiating cells. PI3K inhibition has also been shown to block proliferation of glioma cells.

In spite of great advances in understanding pathways related to cancer and cancer therapy, cancer treatment still goes back to its former modes of treatment which include chemotherapy, surgery, radiation therapy, and the like. These treatment methods are not specific and only partially effective with several side effects.

There is thus always a need for new and more effective and efficient methods of treatment in the world. In fact, there is considerable current interest in developing anticancer agents with novel modes of action because of the development of resistance by cancer cells towards current anticancer drugs and also non-specific toxicity of many current cancer drugs.

Specifically, there is a need to provide a new anticancer treatment that does not cause non-specific toxicity to healthy cells and that is effective in treating and/or curing cancer.

### SUMMARY OF THE INVENTION

The present invention is defined in the appended independent claims. Some optional features of the present invention are defined in the appended dependent claims.

According to one aspect of the present invention, there is provided a use of a fusion protein comprising at least one polypeptide B, which is a Type 1 Ribosome Inactivating Protein (RIP) or fragment thereof; and
(i) at least one polypeptide A which is an antimicrobial peptide; and/or
(ii) at least one polypeptide C which is a Cationic Antimicrobial Peptide (CAP) or fragment thereof

for the preparation of a medicament for treating a tumour and/or cancer in a subject.

In another aspect of the present invention; there is provided the use of the fusion protein according to any aspect of the present invention for the preparation of a medicament for regulating the MHC Class I pathway.

According to a further aspect of the present invention, there is provided a method of treating a tumour and/or cancer in a subject in need thereof comprising a step of administering the fusion protein according to any aspect of the present invention.

According to another aspect of the present invention, there is provided a fusion protein according to any aspect of the present invention for use in the treatment of a tumour and/or cancer in a patient in need thereof.

As will be apparent from the following description, preferred embodiments of the present invention allow for a fusion protein with an optimal effectiveness with a broad spectrum therapy and/or allowing oral delivery of the protein as some of the several applications.

### BRIEF DESCRIPTION OF THE FIGURES

Preferred embodiments of the fusion protein will now be described by way of example with reference to the accompanying figures in which:

FIG. 1 is a translation map of RetroMAD1 (SEQ ID NO:1 and SEQ ID NO:2).

FIG. 2 is a gel image showing A) Time course expression and B) Solubility of RetroMAD1 expression in E. Coli BL21(DE3) cells. Cells harbouring pRMD were harvested before induction (0 h), and after induction for 1 h, 2 h and 3 h represents the pellet phase, the hours with asterisk (*) represents the supernatant phase. Proteins were analysed on a 15% SDS-PAGE. M: PageRuler™ Protein Ladder Fermentas, U: uninduced, IND: induced and IB: purified inclusion bodies. Arrow indicates E. coli produced RetroMAD1 (41.2 kDa)

FIG. 3 is a graph showing the cell number of simultaneously treated normal PBMC at post-72 hours incubation with RetroMAD1.

FIG. 4 is a graph showing the cell number of simultaneously treated Non-Hodgkin's Lymphoma PBMC at post-72 hours incubation.

FIGS. 5A and B are standard curves to determine the concentration of RetroMAD1 in cat serum using capture ELISA.

FIG. 5C is a graph showing the concentration of RetroMAD1 in stomach of guinea pig against time

FIG. 6A is a graph showing the concentration of RetroMAD1 in the serum of control and treated mice derived from capture ELISA.

FIG. 6B is a graph showing the triplicate data confirming the excellent conformity of results used to derive RetroMAD1 concentration in the serum in FIG. 6(A).

FIG. 7A-C are graphs showing the concentration of RetroMAD1 in treated Guinea Pig serum (A), Small Intestine (B) and Stomach (C) against time

FIG. 8A-C are SDS-page results showing Day 1 (A), Day 3 (B), Day 7 (C) and Day 30 (D) thermostability of RetroMAD1.

FIG. 9A is SDS-page results showing the 6th month thermostability of RetroMAD1 in various temperatures.

FIG. 9B is SDS-page results showing the 6th Month thermostability with various temperatures, using β-mercaptoethanol (BME) as reducing agent onto RetroMAD1. In this SDS PAGE, sample of same stock from −20° C. was introduced as a control as well as sample from 4° C.

FIG. 10 is the pathway of antigen processing and presentation.

FIG. 11 are MRI scans of subject before—Dec. 18, 2010 (left) and after—Sep. 5, 2011 (right) RetroMAD1 treatment

FIG. 12A-C are graphs showing the concentration of RetroMAD1 (A), RetroGAD1 (B), Tamapal1 (C) (μg/ml) in mice blood serum after oral administration of RetroMAD1 (A), RetroGAD1 (B), Tamapal1 (C) at 0.5, 1, 2, 4, 8, 12 hours for Day 1 and 30 minutes post feeding for Day 2, Day 3, Day 4, Day 5, Day 6, Day 7 and Day 10.

FIG. 13A-C are graphs showing the concentration of RetroMAD1 (A), RetroGAD1 (B), Tamapal1 (C) (μg/ml) in stomach, liver, intestine and kidney against Time

FIG. 14A-D are images of SDS-page results showing Day 1 (A), Day 7 (B), Day 1 and Day 7 at 50° C. (C) and Day 30 (D) thermostability of RetroGAD1 (temperatures stated on the top of image and the different time points stated on the bottom of the wells). Protein ladder is the molecular weight markers; sample incubated at −20° C. is the control for respective drugs; BME is 2×β-mercaptoethanol, each sample is loaded with (+) or without (−) BME.

FIG. 15A-D are images of SDS-page results showing Day 1 (A), Day 7 (B), Day 1 and Day 7 at 50° C. (C) and Day 30 (D) thermostability of Tamapal1 (temperatures stated on the top of image and the different time points stated on the bottom of the wells). Protein ladder is the molecular weight markers; sample incubated at −20° C. is the control for respective drugs; BME is 2×β-mercaptoethanol, each sample is loaded with (+) or without (−) BME.

FIG. 16A-D are images of results of SDS-page proteolytic digestion of RetroGAD1 with pepsin (pH2), trypsin (pH8) and chymotrypsin (pH8) for 1 hour, 2 hours, 3 hours and 4 hours at 37° C. Sample without presence of enzymes and pre-dissolved RetroGAD1 (stock) were used as negative controls (no digestion). 20 uL of each protein sample with 4× sample buffer was loaded onto SDS-PAGE gels and fragments of protein was analysed.

FIG. 17A-D are images of results of SDS-page proteolytic digestion of Tamapal1 with pepsin (pH2), trypsin (pH8) and chymotrypsin (pH8) for 1 hour, 2 hours, 3 hours and 4 hours at 37° C. Sample without presence of enzymes and pre-dissolved Tamapal1 (stock) were used as negative controls (no digestion). 20 uL of each protein sample with 4× sample buffer was loaded onto SDS-PAGE gels and fragments of protein was analysed.

FIG. 18 are images of results of a SDS-page proteolytic digestion of RetroMAD1 by pepsin (pH2), trypsin (pH8) and chymotrypsin (pH8) for 1 hour, 2 hours and 3 hours at 37° C. Sample without presence of enzymes and pre-dissolved Tamapal1 (stock) were used as negative controls (no digestion). 20 uL of each protein sample with 4× sample buffer was loaded onto SDS-PAGE gels and fragments of protein was analysed.

FIG. 19 is a graph showing the percentage of viral reduction by RetroGAD1, RetroMAD1 and Tamapal1 in simultaneous treatment at 72 h determined by PCR.

FIG. 20A-C are graphs showing the inhibition of NS2B-NS3 polyprotein protease by RetroMAD1 (A) RetroGAD1 (B), and Tamapal1 (C).

FIG. 21 is a graph showing the percentage of viral reduction caused by RetroGAD1, RetroMAD1 and Tamapal1 in simultaneous treatment at 72 h determined by PCR.

FIG. 22A-B are graphs showing cell viability of HepG2 when treated with Tamapal1 (A) compared with normal cell lines such as Vero, RWPE and 184B5 and when treated with RetroGAD1 (B) compared with normal cell lines such as Vero, RWPE, 184B5 and PBMC.

FIG. 23 is a graph showing cell viability of PC3 prostate cancer cell line when treated with Tamapal1 compared against normal prostate cell line RWPE.

FIG. 24 is a graph showing cell viability of HepG2 liver cancer cell line when treated with K5 compared with normal Vero cells.

FIGS. 25 A and B are plots depicting the treatment of HepG2 cells with RetroGAD1 with concentration of 30 μg/m1 (A) and treatment of prostate cancer PC3 cells with Tamapal1 at 5 μg/ml (B). The results showed that Tamapal1 did not induce the caspase pathway

FIG. 26A-C are plots showing the percentage of inactivation of the PI3 Kinase by K5 at 5 82 g/ml (A), 13 μg/ml (B) and 40 μg/ml (C).

FIG. 27A-C are plots showing the percentage of inactivation of the PI3 Kinase by Tamapal1 at 5 μg/ml (A), 15 μg/ml (B) and 30 μg/ml (C).

FIG. 28 is a plot showing the percentage of inactivation of the PI3 Kinase by RetroGAD1 at 15 μg/ml.

FIG. 29 is a plot showing that 19.48% of MAPK pathway is inactivated in total population of HepG2 treated with RetroGAD1 at 30 μg/ml.

FIG. 30 is a plot showing that 36% of EGFR pathway is inactivated in total population of HepG2 treated with RetroGAD1 at 30 μg/ml

FIG. 31 is a plot showing that 3.54% of EGFR pathway is inactivated in total population of HepG2 treated with K5 at 5 μg/ml

FIG. 32 are images of HepG2 cells untreated (A) and treated with 7 μg/ml of RetroGAD1(B), where the cells are lysed.

FIG. 33 are images of PC3 cells untreated (A) and treated with 5 μg/ml of Tamapal1 (B),

FIG. 34 are images of vero cells untreated (A) and treated with 20 μg/ml of Tamapal1 (B) (The IC₅₀ of Tamapal1 on vero cells),

FIG. 35 are images of HepG2 cells untreated (A) and treated with 20 μg/ml of Tamapal1 (B) which showed cytotoxicity effects. Cells morphology and integrity compared to the control has changed looking circular and less intact.

FIG. 36 is a schematic diagram of the mechanism of Tamapal1 on cancer

FIG. 37 is a gel image of a protein profile of RetroMAD1 against HSV2; cells as control, cells treated with RetroMAD1, Cells infected with HSV2 and HSV2 infected cells treated with RetroMAD1

FIG. 38 is a schematic diagram of the pathway of HSV2 infection in cells (i) Entry (ii) Uncoating and nuclear transport (iii) Replication (iv) Translation (v) Transport to cytoplasm and (vi) Egress. Proteins involved are mainly in viral entry, replication and translation.

FIG. 39 a gel image of a protein profile of RetroGAD1, Tamapal1 and K5 against HSV2; cells as control, cells treated with RetroGAD1, Tamapal1 and K5, Cells infected with HSV2 and HSV2 infected cells treated with RetroGAD1, Tamapal1 and K5.

### DETAILED DESCRIPTION OF THE INVENTION

For convenience, certain terms employed in the specification, examples and appended claims are collected here.

The term “adjuvant”, as used in the context of the invention refers to an immunological adjuvant.

By this, an adjuvant is meant to be a compound that is able to enhance or facilitate the immune system's response to the ingredient in question, thereby inducing an immune response or series of immune responses in the subject. The adjuvant can facilitate the effect of the therapeutic composition by forming depots (prolonging the half-life of the ingredient), provide additional T-cell help and stimulate cytokine production. Facilitation of antigen survival and unspecific stimulation by adjuvants may, in some cases, be required if the antigenic molecule are only weakly antigenic or only exerts weak to moderate interactions with compounds, molecules, or cells of the immune system.

The term “analogue” as used in the context of the invention refers to a peptide that may be modified by varying the amino acid sequence to comprise one or more naturally-occurring and/or non-naturally-occurring amino acids, provided that the peptide analogue is capable of reducing or preventing growth of a tumour or cancer. For example, the term “analogue” encompasses an inhibitory peptide comprising one or more conservative amino acid changes. The term “analogue” also encompasses a peptide comprising, for example, one or more D-amino acids. Such an analogue has the characteristic of, for example, protease resistance. Analogues also include peptidomimetics, e.g., in which one or more peptide bonds have been modified. Preferred analogues include an analogues of a peptide as described according to any embodiment here comprising one or more non-naturally-occurring amino acid analogues.

The terms “anticancer” or “antitumour” may be used interchangeably and as used in the context of the invention refers to the biological activity of a peptide or analogue or derivative thereof of the present invention, and means that the proteins of the present invention has the capacity to destroy, disrupt proliferation or otherwise reduce tumour or cancerous growth in a subject in need thereof. The peptide or analogue or derivative thereof of the present invention is capable of destroying a tumour or cancer and/or reducing or preventing growth of a tumour or cancer i.e., the peptide may have chemotherapeutic activity and/or antineoplastic activity. The peptide may be a drug, compound or molecule, which includes the fusion protein according to any aspect of the present invention for use in treating tumour or cancer. Methods for determining anticancer activity of a peptide or analogue or derivative thereof will be apparent to a skilled person and/or described herein. For example, the peptide or analogue or derivative is applied to a substrate upon which a tumour or cancerous growth or cell lines and, after a suitable period of time, the level of growth inhibition and/or cell death of tumour or cancer cell is determined.

The term “comprising” as used in the context of the invention refers to where the various components, ingredients, or steps, can be conjointly employed in practicing the present invention. Accordingly, the term “comprising” encompasses the more restrictive terms “consisting essentially of” and “consisting of.” With the term “consisting essentially of” it is understood that the epitope/antigen of the present invention “substantially” comprises the indicated sequence as “essential” element. Additional sequences may be included at the 5′ end and/or at the 3′ end. Accordingly, a polypeptide “consisting essentially of” sequence X will be novel in view of a known polypeptide accidentally comprising the sequence X. With the term “consisting of” it is understood that the polypeptide, polynucleotide and/or antigen according to the invention corresponds to at least one of the indicated sequence (for example a specific sequence indicated with a SEQ ID Number or a homologous sequence or fragment thereof).

The term “derivative” as used in the context of the invention includes e.g., a fragment or processed form of the stated peptide, a variant or mutant comprising one or more amino acid substitutions, deletions of additions relative to the stated peptide, a fusion protein comprising the stated peptide or a peptide comprising one or more additional non-peptide components relative to the stated peptide e.g., a chemical component, e.g., polyethylene glycol (PEG). The term “derivative” also encompasses polypeptides comprising the fusion protein according to the invention. For example, the polypeptide comprises a label, such as, for example, an epitope, e.g., a FLAG epitope or a V5 epitope or an HA epitope. For example, the epitope is a FLAG epitope. Such a tag is useful for, for example, purifying the polypeptide. A preferred derivative of an antitumour or anticancer fusion protein of the invention has enhanced stability. For example, a cleavage site of a protease active in a subject to which a fusion protein is to be administered is mutated and/or deleted to produce a stable derivative of an antitumour or anticancer fusion protein of the invention. The term “derivative” also encompasses a derivatized peptide, such as, for example, a peptide modified to contain one or more-chemical moieties other than an amino acid. The chemical moiety may be linked covalently to the peptide e.g., via an amino terminal amino acid residue, a carboxy terminal amino acid residue, or at an internal amino acid residue. Such modifications include the addition of a protective or capping group on a reactive moiety in the peptide, addition of a detectable label, and other changes that do not adversely destroy the activity of the peptide compound.

Accordingly, acceptable amino acid substitutions are generally therefore based on the relative similarity of the amino acid side-chain substituents, for example, their hydrophobicity, hydrophilicity, charge, size, and the like. Exemplary substitutions which take several of the foregoing characteristics into consideration are well known to those of skill in the art and include: arginine and lysine; glutamate and aspartate; serine and threonine; glutamine and asparagine; and valine, leucine and isoleucine. The isolated peptides of the present invention can be prepared in a number of suitable ways known in the art including typical chemical synthesis processes to prepare a sequence of polypeptides.

The term “fragment” as used in the context of the invention refers to an incomplete or isolated portion of the full sequence of the fusion protein according to any aspect of the present invention which comprises the active site(s) that confers the sequence with the characteristics and function of the protein. In particular, it may be shorter by at least one amino acid. For example a fragment of the fusion protein according to the present invention comprises the active site(s) that enable the protein to recognise an aberrant cell such as a tumour cell or cancer cell. The fragment may at least be 10 amino acids in length. For example, a non-limiting fragment of RIP may at least comprise the core or the bioactive site of the RIP which may be approximately 5 kDa in size.

The term “fusion protein(s)” as used in the context of the invention refers to proteins created through the joining of two or more genes, which originally coded for separate proteins. Translation of this fusion gene results in a single polypeptide with functional properties derived from each of the original proteins. Recombinant fusion proteins are created artificially by recombinant DNA technology for use in biological research or therapeutics. For example, the fusion protein according to any aspect of the present invention may comprise a polypeptide B; and a polypeptide C which is a CAP. The fusion protein may have anticancer properties. The fusion protein according to any aspect of the present invention may further comprise a polypeptide A and/or a polypeptide D. Each individual part and/or the whole the fusion protein may have anticancer properties. For example, polypeptide A, B, C and/or D may have anticancer properties. As a whole A-B-C and/or A-B-C-D may have anticancer properties. The structure of the fusion protein may be A-B-C, A-C-B, C-A-B, C-B-A, B-A-C, B-C-A, A-B-C-C, A-B, B-C, B-C-C, C-C-B-C-C, C-B-C, C-B-D, C-D-B, B-D-C, B-C-D, D-C-B or D-B-C. In particular, the fusion protein may comprise dimers and/or tandem repeats. More in particular, the structure of the fusion protein according to any aspect of the present invention may be repeats of the structure mentioned above. For example, the structure may be A-A-B-C-C, C-C-B-C-C, A-A-B-A-A and the like. The polypeptide A, B or C in each fusion protein may be the same protein or may be a different protein when repeated. Polypeptide A may be theta defensin, an analogue, or a fragment thereof. A fusion protein according to the present invention may comprise the sequence of SEQ ID NO:1, a variant, derivative or fragment thereof. The term “RetroMAD1” is used in the present invention to refer to a fusion protein with the structure A-B-C and with amino acid sequence SEQ ID NO:1. In particular, in RetroMAD1 polypeptide A may be Retrocyclin 101, polypeptide B may be MAP30 and polypeptide C may be Dermaseptin 1. These peptides may be directly fused to one another or connected to one another by a linker peptide.

The term “linker peptide”, as used in the context of the invention is used interchangeably with the term “linker” herein. A linker peptide is a peptide that covalently or non-covalently connects two or more molecules or peptides, thereby creating a larger complex consisting of all molecules or peptides including the linker peptide. A non-limiting example of a linker peptide may be SEQ ID NO:3 and/or SEQ ID NO:27.

The term “polypeptide” as used in the context of the invention may refer to a long, continuous, and unbranched peptide and may include cyclic polypeptides. Proteins consist of one or more polypeptides arranged in a biologically functional way and may often be bound to cofactors, or other proteins. In particular, the protein according to any aspect of the present invention may be naturally occurring, de novo and/or synthetic.

The terms “subject”, “patient” and “individual” are used interchangeably and are used in the context of the invention refers to either a human or a non-human animal. These terms include mammals, such as humans, primates, livestock animals (including bovines, porcines, etc.), companion animals (e.g. canines, felines, etc) and rodents (e.g. mice and rats). In particular, the subject is a human that may develop a tumour or cancer against which a fusion protein analogue or derivative of the invention is cytotoxic.

The term “treating”, as used in the context of the invention refers to reversing, alleviating, or inhibiting the progress of a tumour or cancerous growth. The term “treatment”, as used in the context of the invention may also refer to prophylactic, ameliorating, therapeutic or curative treatment.

The term “tumour” or “cancer”, as used in the context of the invention refers to an abnormal mass of tissue as a result of abnormal proliferation of cells. The term “tumour” refers to a mass of cells which may not necessarily be cancer. Cancer is a type of malignant tumour. The term “tumour” or “cancer” as used herein may be used to describe a disease selected from the group consisting of Non-Hodgkin's Lymphoma, brain, lung, colon, epidermoid, squamous cell, bladder, gastric, pancreatic, breast, head, neck, renal, kidney, liver, ovarian, prostate, colorectal, uterine, rectal, oesophageal, testicular, gynecological, thyroid cancer, melanoma, hematologic malignancies such as acute myelogenous leukemia, multiple myeloma, chronic myelogneous leukemia, myeloid cell leukemia, glioma, pontine glioblastoma, Kaposi's sarcoma, or any other type of solid or liquid cancer.

The term “variant”, as used in the context of the invention can alternatively or additionally be characterised by a certain degree of sequence identity to the parent polypeptide from which it is derived. More precisely, a variant in the context of the present invention exhibits at least 30% sequence identity, in particular at least 40%, 50%, 60%, 70%, 80% or 90% sequence identity. More in particular, a variant in the context of the present invention exhibits at least 95% sequence identity to its parent polypeptide. The variants of the present invention exhibit the indicated sequence identity, and preferably the sequence identity is over a continuous stretch of 100, 150, 200, 300, 315, 320, 330, 340, 344 or more amino acids. The similarity of nucleotide and amino acid sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL available e.g. on http://www.ebi.ac.uk/Tools/clustalw/. Preferred parameters used are the default parameters as they are set on http://www.ebi.ac.uk/Tools/clustalw/ or http://www.ebi.ac.uk/Tools/clustalw2/index.html. The grade of sequence identity (sequence matching) may be calculated using e.g. BLAST, BLAT or BlastZ (or BlastX). Preferably, sequence matching analysis may be supplemented by established homology mapping techniques like Shuffle-LAGAN (Brudno M., Bioinformatics 2003b, 19 Suppl 1:154-162) or Markov random fields. When percentages of sequence identity are referred to in the present application, these percentages are calculated in relation to the full length of the longer sequence, if not specifically indicated otherwise.

A person skilled in the art will appreciate that the present invention may be practiced without undue experimentation according to the method given herein. The methods, techniques and chemicals are as described in the references given or from protocols in standard biotechnology and molecular biology textbooks.

In one aspect of the present invention, there is provided the use of at least one fusion protein comprising at least one polypeptide B, which is a Ribosome Inactivating Protein (RIP) or fragment thereof; and
(i) at least one polypeptide A which is an antimicrobial peptide; and/or
(ii) at least one polypeptide C which is a Cationic AntiMicrobial Peptide (CAP) or a fragment thereof,

for the preparation of a medicament for treating a tumour or cancer in a subject in need thereof.

In another aspect of the present invention, there is provided the use of the fusion protein according to any aspect of the present invention for the preparation of a medicament for regulating the MHC Class I pathway. The MHC class I pathway regulation may involve the upregulation of at least one gene associated with an antigen presenting cell.

In particular, the fusion protein further comprises at least one polypeptide D, which is a synthetic anticancer polypeptide, or a fragment thereof.

The fusion protein according to any aspect of the present invention may be an anticancer compound capable of a broad spectrum of applications and that may be economically produced without any limitation of raw material supply unlike certain anticancer compounds known in the art. The fusion protein according to any aspect of the present invention may thus be economically produced in a large scale without any limitations of raw material supply.

In order to achieve broad-spectrum activity, the fusion peptide according to any aspect of the present invention may be able to interfere with tumour and/or cancer cell growth or proliferation in a number of different pathways, that is to say, in cell division or DNA synthesis. The fusion may thus have a multi-domain and/or multifunctional ability. An entire new class of anticancer drugs may thus be produced from the fusion protein according to any aspect of the present invention. The number of combinations and permutations that may be obtained from expressed polypeptides A, B, C and D as fusion antitumour or anticancer proteins potentially numbers in the tens of thousands.

The use of the fusion proteins according to any aspect of the present invention, involve combining anticancer properties from 2, or more likely 3 genes, to produce potent anticancer chimeric proteins that are capable of oral administration and are stable at room temperature to avoid costly cold-chain transportation. Also, the fusion products according to any aspect of the present invention may have potent antiviral activities that can be useful a significant percentage of human cancers are caused by viral infections. In particular, these fusion products may be capable of inhibition of polyprotein serine proteases as demonstrated by their inhibition of the NS2B NS3 protease of another Flavivirus i.e. that of Dengue Virus. Also, these fusion products may be capable of killing HSV-2 as shown in the Examples.

In particular, the fusion protein may comprise at least one formula selected from the group consisting of formulas I-XIX:

A-B-C, Formula I

A-B-C-C, Formula II

A-B, tm Formula III

A-C-B, Formula IV

C-A-B, Formula V

C-B-A, Formula VI

C-B, Formula VII

B-A-C, Formula VIII

B-A-C-C, Formula IX

B-C-A, Formula X

B-C, Formula XI

B-A, Formula XII

C-C-B-C-C, Formula XIII

C-B-C, Formula XIV

C-B-D, Formula XV

B-C-D, Formula XVI

B-D-C, Formula XVII

D-C-B, Formula XVIII

D-B-C Formula XIX

B-D, Formula XX

D-B. Formula XXI

Polypeptide A may be an antimicrobial peptide. In particular, polypeptide A may be an viral entry inhibitory protein. More in particular, polypeptide A may be a defensin, an analogue, or a fragment thereof. Even more in particular, the defensin may be an alpha, a beta, a theta or a big defensin, an analogue, or a fragment thereof, polypeptide B may be Type 1 RIP, or a fragment thereof, polypeptide C may be Cationic AntiMicrobial Peptide (CAP), or a fragment thereof, polypeptide D may be synthetic anticancer sequence; and—may be a direct linkage or a linker peptide.

In particular, the linker peptide may comprise a polypeptide sequence: [VPXVG]ₙ, (SEQ ID NO:3) wherein X is an unknown or other amino acid and n is the number of repeats of SEQ ID NO:3 in each linker peptide. For example, n may be 1, 2, 3, 4 or 5. More in particular, X in SEQ ID NO:3 is G and n is 2.

In another example, the linker peptide may be a glycine-serine linker. In particular, the glycine-serine linker may have a sequence of [G-G-G-S]ₙ (SEQ ID NO:27).

In particular, the fusion protein may comprise the formula I:

A-B-C-

wherein, polypeptide A is a defensin (α, β, θ or big) an analogue, or a fragment thereof. In particular, polypeptide A may be a theta defensin, an analogue, or a fragment thereof, polypeptide B is Type 1 RIP, or a fragment thereof, and polypeptide C may be CAP, or a fragment thereof and—may be a direct linkage or a linker peptide.

More in particular, polypeptide A may be fused to polypeptide B via at least one first linker peptide of SEQ ID NO: 3. Even more in particular, polypeptide A may be fused to polypeptide B via a peptide of SEQ ID NO: 3, wherein X is G and n is 2. Polypeptide B may be directly linked to polypeptide C with no linker peptide in-between. Polypeptide C in formula I may comprise a second linker peptide on the free end not linked to B. The second linker peptide may comprise the formula SEQ ID NO: 3. Even more in particular, in the second linker peptide X is G and n is 2.

Polypeptide A may be an viral entry inhibitor protein. In particular, polypeptide A may be a defensin (α, β, θ or big). Defensins are known to be up-regulated in tumors and exhibit anti-angiogenic antitumor effects. In particular, polypeptide A may be a theta Defensin of a vertebrate or invertebrate origin. In particular, theta Defensin may be from a bacterium, fungus, mammal, amphibian or reptile. The mammal may be a non-human primate and/or the invertebrate may be a Horseshoe crab and/or an insect. The theta Defensin may be selected from the group consisting of Rhesus minidefensin (RTD-1), RTD-2, RTD-3, Retrocyclin-1, Retrocyclin-2, Retrocyclin-3 from Macaca mulatta of SEQ ID Nos: 7-12 respectively and the like (Tang Y Q, 1999; Leonava L, 2001; Wang W, 2004).

The theta Defensin may be synthetic and may be selected from a group of retrocyclin congeners RC100-RC108 and RC110-RC114 of SEQ ID NO:13-25 respectively (Cole et. al. 2002: PNAS, V99(4):1813-1818; Wang et. al. 2003: J. Immunol. 170:4708-4716). The sequences of Retrocyclin (RC) 100-108 and RC110-RC114 are shown in Table 1a below.

TABLE 1a
Polypeptide sequences of naturally
occurring and synthetic theta
Defensin proteins.

| SEQ | |
|---|---|
| ID | |
| NO: | Sequences |
| 7 | GFCRCLCRRGVCRCICTR |
| 8 | RCLCRRGVCRCLCRRGVC |
| 9 | RCICTRGFCRCICTRGFC |
| 10 | GICRCICGRGICRCICGR |
| 11 | GICRCICGRGICRCICGR |
| 12 | RICRCICGRRICRCICGR |
| 13 | GICRCICGRGICRCICGR |
| 14 | GICRCICGKGICRCICGR |
| 15 | GICRCYCGRGICRCICGR |
| 16 | GICRCICGRGICRCYCGR |
| 17 | GYCRCICGRGICRCICGR |
| 18 | GICRCICGRGYCRCICGR |
| 19 | GICYCICGRGICRCICGR |
| 20 | GICICICGYGICRCICGR |
| 21 | GICICICGRGICYCICGR |
| 22 | GICICICGRGICYCICGR |
| 23 | RGCICRCIGRGCICRCIG |
| 24 | RGCICRCIGRGCICRCIG |
| 25 | GICRCICGRGICRCICGR |
| 26 | GICRCICGKGICRCYCGR |

Polypeptide A may be an alpha-defensin selected from the group consisting of human neutrophil protein 1 (HNP-1), HNP-2, HNP-3, HNP-4, Human defensin 5 and Human defensin 6, an analogue, or a fragment thereof. The alpha defensin may be from mice, monkeys, rats, rabbits, guinea pigs, hamster, horse, elephant, baboon, hedgehog, horse, chimpanzee, orangutan, macaque, marmoset and the like from any mammalian origin.

In another example, the polypeptide A may be a beta-defensin selected from the group consisting of DEFB 1, DEFB 4A, DEFB 4B, DEFB 103A, DEFB 103B, DEFB 104A, DEFB 104B,

DEFB 105A, DEFB 105B, DEFB 106A, DEFB 106B, DEFB 107A, DEFB 107B, DEFB 108B, DEFB108 P1-4, DEFB 109 P1, DEFB 109 P1B, DEFB 109 P2-3, DEFB 110, DEFB 112-119, DEFB 121-136 and the like from any mammalian origin.

Polypeptide A may be a Big defensins originating from (i) Amphioxus—Branchiostoma florida and Branchiostoma belched; (ii) Horseshoecrab—Tachypleus tridentatus; (iii) Mussel—Mytilus galloprovincialis; (iv) Clam—Ruditapes philippinarum, (v) Oyster—Crassostrea gigas and the like from any arthropod origin.

Polypeptide B may be a Type 1 Ribosome Inactivating Protein selected from the group consisting of Ebulitins, Nigritins, Amarandins, Amaranthus antiviral/RIP, Amaranthin, Atriplex patens RIP, Beta vulgaris RIP, β-vulgin, Celosia cristata RIP, Chenopodium album RIP, CAP30B, Spinacea oleracea RIP, Quinqueginsin, Asparins, Agrostin, Dianthins, DAPs, Dianthus chinensis', Lychnin, Petroglaucin, Petrograndin, Saponaria ocymoides RIP, Vacuolas saporin, Saporins, Vaccaria hispanica RIP, Benincasins, Hispin, Byrodin's, Colocins, Cucumis figarei RIP, Melonin, C. moschata RIP, Cucurmosin, Moschatins, Pepocin, Gynostemmin, Gynostemma pentaphyllum RIP, Gypsophilin, Lagenin, Luffaculin, Luffangulin, Luffin, MORs, Momordin II, Momorcharin's, Momorcochin, Momorcochin-S, Sechiumin, Momorgrosvin, Trichoanguin, Kirilowin, α-trichosanthin, TAP-29, Trichokirin, Trichomislin, Trichosanthin, Karasurin, Trichomaglin, Trichobakin, Crotin, Euserratin Antiviral Protein GAP-31, Gelonin, Hura crepitans RIP, Curcin, Jathropa curcas RIP, Mapalmin, Manutins, α-pisavin, Charibdin, Hyacinthus orientalis RIP, Musarmin, Iris hollandica RIP, Cleroendrum aculeatum RIP, CIPs,) Crip-31, Bouganin, Bougainvilla spectbilis RIP, Bougainvillea×buttiana Antiviral protein 1 (BBAP1), Malic enzymes, MAP-S, pokeweed antiviral proteins (PAP), PD-SI, DP-S2, Dodecandrin, PIP, PIP2, Phytolacca octandra anti-viral proteins, Hordeum vulgare RIPs, Hordeum vulgare sub sp. Vulgare Translational inhibitor II, Secale cereale RIP, Tritin, Zea diploperemis RIPs, Malus×domestica RIP, Momordica Anti-HIV Protein, Gelonium multiflorum, Mirabilis expansa 1, phage MU1, betavulgin (Bvg), curcin 2, saporin 6, Maize RIP (B-32), Tobacco RIP (TRIP), Beetins, Mirabilis antiviral protein (MAP), Trichosanthin (TCS), luffins, Momorcharins, Ocymoidin, Bryodin, Pepopsin, β-trichosanthin, Camphorin, YLP, Insularin, Barley RIP, Tritins, Lamjarin, and Volvariella volvacea RIP and the like from any plant origin.

Polypeptide C may be selected from the group consisting of Cyclotides, Siamycins, NP-06, Gramicidin A, Circulins, Kalatas, Ginkbilobin, Alpha-Basrubin, Lunatusin, Sesquin, Tricyclon A, Cycloviolacins, Polyphemusins, hfl-B5, Protegrins (Pig Cathelicidin), Rat Defensins, Human β-defensins, Temporins, Caerins, Ranatuerins, Reptile Defensin, Piscidin's, Lactoferricin B, Rabbit Neutrophils, Rabbit α-Defensin, Retrocyclins, Human α-Defensins, Human β-defensin III (HBD3), Rhesus minidefensin (RTD-1, θ-defensin), rhesus θ-defensins, Human neutrophil peptides, Cecropin As, Melittin, EP5-1, Magainin 2s, hybrid (CE-MA), hepcidin TH1-5, Epinecidin-1, Indolicidin, Cathelicidin-4, LL-37 Cathelicidin, Dermaseptins, Maximins, Brevinins, Ranatuerins, Esculentins, Maculatin 1.3, Maximin H5 and Piscidins, Mundticin KS Enterocin CRL-35, Lunatusin, FK-13 (GI-20 is a derivative), Tachyplesins, Alpha-MSH, Antiviral protein Y3, Palustrin-3AR, Ponericin L2, Spinigerin, Melectin, Clavanin B, Cow cathelicidin's, Guinea pig cathelicidin CAP11, Sakacin 5X, Plectasin, Fungal Defensin, GLK-19, lactoferrin (Lf) peptide 2, Alloferon 1, Uperin 3.6, Dahlein 5.6, Ascaphin-8, Human Histatin 5, Guineapig neutrophils, Mytilins, EP5-1,Hexapeptide (synthetic) Corticostatin IV Rabbit Neutrophil 2, Aureins, Latarcin, Plectasin, Cycloviolins, Vary Peptide E, Palicourein, VHL-1, Gaegurin 5, Gaegurin 6 and the like (U.S. Pat. No. 8,076,284 B2; Kim, S. et al, Peptides, 2003, 24, 945-953).

In particular, polypeptide C may be Gaegurin 5, Gaegurin 6, their analogues, derivatives or fragments thereof, which may have pro-apoptotic properties that may act upon drug sensitive and multidrug resistant tumour cell lines.

Polypeptide D may be bi-functional peptides i.e. 2-domain fusion molecules that act on 2 separate active sites. Polypeptide D may be pro-apoptotic peptide. In particular, polypeptide D may be a Bax-derived membrane-active peptide. Bax-derived membrane-active peptides are apoptosis-inducing peptides that may be capable of causing apoptosis in cancer cells. For example, polypeptide D may be (KLAKLAK)2, SSX2, D-K₄R₂L₉ (Hoskin D. W. et al, 2008), p18 (Tang C et al, 2010) and the like.

In particular, (KLAKLAK)2 may be conjugated with leukemia cell differentiating peptide motifs; with bcl-2 antisense oligonucleotides targeting mitochondrial outer membrane permeability; to αᵥ β₃ integrin receptors targeting endothelial cell apoptosis; to self-assembling cylindrical nanofibres targeting breast cancer cells and to CGKRK glioblastoma-homing peptide motifs together with (KLAKLAK)2 being coated on iron oxide ‘nanoworms’. More particularly, (KLAKLAK)2 may be conjugated with MAP30.

A Cationic Antimicrobial Peptide (CAP) may be an anti-microbial CAP that may have anticancer and/or antiviral properties. CAPs may be a maximum of 100 amino acids in length. CAPs may either be a naturally occurring CAP with sequence with reported anticancer properties or a synthetic CAP sequence with anticancer properties. CAPs may mostly be of animal origin. However, there may also be CAPs, which are from plants, which include but are not limited to cyclotides. For example, bacteria CAPs may include but are not limited to Siamycin, NP-06 and Gramicidin A. Plant CAPs may include Circulin A, B, Kalata B1 and B8; Plant CAPs which may function as entry inhibitors may include Kalata B8, Ginkbilobin, Alpha-Basrubin, Lunatusin and Sesquin, Circulin A, C and D, Tricyclon A and Cycloviolacin H4. Animal CAPs may include Polyphemusin I and II, hfl-B5, Protegrin (Pig Cathelicidin), Rat Defensin NP1, NP2, NP3 and NP4, Human β-defensin I and II, Temporin A, Temporin-LTc, Temporin-Pta, Caerin 1.1, Ranatuerin 6 and 9, Reptile Defensin and Piscidin 1 and 2, Lactoferricin B, Rabbit Neutrophil-1 Corticostatin III a, Rabbit Neutrophil-3A, Rabbit α-Defensin, Retrocyclin-1, Retrocyclin-2, Retrocyclin-3, Human α-Defensin HNP-1, 2, 3,4,5 & 6, Human β-defensin III (HBD3), Rhesus minidefensin (RTD-1,θ-defensin), RTD-2 rhesus θ-defensin, RTD-3 rhesus θ-defensin, Human neutrophil peptide-2, Human neutrophil peptide-3 and human neutrophil peptide-4, Cecropin A, Melittin, EP5-1, Magainin 2, hepcidin TH1-5, and Epinecidin-1, Indolicidin, Cathelicidin-4, Human neutrophil peptide-1, LL-37 Cathelicidin, Dermaseptin-S1, S4 and S9, Maximin 1, 3, 4 and 5, Brevinin 1, Ranatuerin 2P, 6 and 9 Esculentin 2P, Esculentin-1 Arb, Caerin 1.1, 1.9 and 4.1, Brevinin-2-related, Maculatin 1.3, Maximin H5 and Piscidin 1 and 2. Other CAPs may include Mundticin KS Enterocin CRL-35, Lunatusin, FK-13 (GI-20 is a derivative), Tachyplesin I, Alpha-MSH, Antiviral protein Y3, Piscidin 3, Palustrin-3AR, Ponericin L2, Spinigerin, Melectin,

Clavanin B, Cow cathelicidin BMAP-27, BMAP-28, Guinea pig cathelicidin CAP11, Sakacin 5X, Plectasin, Fungal Defensin, GLK-19, lactoferrin (Lf) peptide 2, Kalata B8, Tricyclon A, Alloferon 1, Uperin 3.6, Dahlein 5.6, Ascaphin-8, Human Histatin 5, Guineapig neutrophil CAP2 & CAP1, Mytilin B & C, EP5-1, and Hexapeptide (synthetic) Corticostatin IV Rabbit Neutrophil 2.

Cationic antimicrobial peptides (CAP) may exhibit cytotoxic activity against cancer cells as the electrostatic attraction between negatively charged components of cancer cells are attracted to positively charged CAPs resulting first in binding and then further on in cell disruption. Cancer cells may carry a net negative charge due to over-expression of phosphatidylserine, O-glycosylated mucins and heparin sulphate. Furthermore, cancer cells may have increased numbers of microvilli leading to an increase in cell surface area, which may in turn enhance their vulnerability to CAP action. CAPs are also known for various antiviral properties and some of them also possess anticancer properties.

The Type 1 RIP may:
(i) act as a pro-apoptotic polypeptide which upregulate pro-apoptotic genes that may include but not limited to caspase-12, Bax and the like, or downregulate anti-apoptotic gene including but not limited to BcI-2 and the like in tumour or cancer cells (Fan, J-M., et al, Mol Biotechnol, 2008, 39, 79-86);
(ii) act as a DNA glycosylase/apurinic (AP) lyase capable of irreversibly relaxing tumour or cancer cell supercoiled DNA and catalyzing double-stranded breakage to form inactive products;
(iii) act in alternative cytochrome patways as well as Mn²⁺ and Zn²⁺ interactions with negatively charged surfaces next to catalytic sites, facilitating DNA substrate binding instead of directly participating in catalysis (Wang et al, Cell, 1999, 99, 433-442);
(iv) as an RNA N-Glycosidase which hydrolyses the N-C glycosidic bond of adenosine at position 4324 of the universally conserved sarcin/ricin domain(S/R domain) of the 28S-rRNA in the eukaryotic ribosome and render it incapable of carrying out protein synthesis thus, functionally, blocking translation.

In particular, the type 1 RIP may be selected from the group consisting of α-Ebulitin, β-Ebulitin, γ-Ebulitin, Nigritin f1, Nigritin f2, Amarandin-S, Amaranthus antiviral/RIP, Amarandin-1, Amarandin-2, Amaranthin, Atriplex patens RIP, Beta vulgaris RIP, β-vulgin, Celosia cristata RIP, Chenopodium album RIP, CAP30B, Spinacea oleracea RIP, Quinqueginsin, Asparin 1, Asparin 2, Agrostin, Dianthin 29, DAP-30, DAP-32, Dianthin 30, Dianthus chinensis RIP1, Dianthus chinensis RIP2, Dianthus chinensis RIP3, Lychnin, Petroglaucin, Petrograndin, Saponaria ocymoides RIP, Vacuolas saporin, Saporin-1, Saporin-2, Saporin-3, Saporin-5, Saporin-6, Saporin-7, Saporin-9, Vaccaria hispanica RIP, Benincasin, α-benincasin, β-benincasin, Hispin, Byrodin I, Byrodin II, Colocin I, Colocin 2, Cucumis figarei RIP, Melonin, C. moschata RIP, Cucurmosin, Moschatin, Moschatin I, Moschatin II, Moschatin III, Moschatin IV, Moschatin V, Pepocin, Gynostemmin I, Gynostemmin II, Gynostemmin III, Gynostemmin IV, Gynostemmin V, Gynostemma pentaphyllum RIP, Gypsophilin, Lagenin, Luffaculin, Luffangulin, Luffin-alpha, Luffin-B, MOR-I, MOR-II, Momordin II, Alpha-momorcharin, β-momorcharin, γδ-momorcharin, γ-momorcharin, Momorcochin, Momorcochin-S, Sechiumin, Momorgrosvin, Trichoanguin, α-kirilowin, β-kirilowin, α-trichosanthin, TAP-29, Trichokirin, Trichomislin, Trichosanthin, Karasurin-A, Karasurin-B, Trichomaglin, Trichobakin, Crotin 2, Crotin 3, Euserratin 1, Euserratin 2, Antiviral Protein GAP-31, Gelonin, Hura crepitans RIP, Curcin, Jathropa curcas RIP, Mapalmin, Manutin 1, Manutin 2, α-pisavin, Charibdin, Hyacinthus orientalis RIP, Musarmin 1, Musarmin 2, Musarmin 3, Musarmin 4, Iris hollandica RIP, Cleroendrum aculeatum RIP, CIP-29, CIP-34, Crip-31, Bouganin, Bougainvilla spectbilis RIP, Bougainvillea×buttiana Antiviral protein 1 (BBAP1), malic enzyme 1 (ME1), ME2, MAP-S, pokeweed antiviral protein (PAPa-1), PAPa-2, PAP-alpha, PAP-I, PAP-II, PAP-S, PD-SI, DP-S2, Dodecandrin, Anti-viral protein PAP, PIP, PIP2, Phytolacca octandra anti-viral protein, Phytolacca, octandra anti-viral protein II, Hordeum vulgare RIP-I, Hordeum vulgare RIP-II, Hordeum vulgare sub sp. Vulgare Translational inhibitor II, Secale cereale RIP, Tritin, Zea, diploperemis RIP-I, Zea diploperemis RIP-II, Malus×domestica RIP, Momordica Anti-HIV Protein (MAP30), Gelonium multiflorum (GAP31), pokeweed antiviral protein (PAP), Mirabilis expansa 1 (ME1), malic enzyme 2 (ME2), Bougainvillea×buttiana antiviral protein 1 (BBAP1), phage MU1, betavulgin (Bvg), curcin 2, saporin 6, Maize RIP (B-32), Tobacco RIP (TRIP), beetin (BE), BE27, Mirabilis antiviral protein (MAP), Trichosanthin (TCS), α-luffin, α-Momorcharin (α-MMC), β-MMC luffin, Ocymoidin, Bryodin, Pepopsin, β-trichosanthin, Camphorin, YLP, Insularin, Barley RIP, Tritins, Lamjarin, and Volvariella volvacea RIP and the like from any plant origin.

MAP30 polypeptide or Ribosomal Inactivating Protein may act in a pro-apoptotic manner to destroy tumour or cancer cells selectively. In particular, MAP30 polypeptide may be selectively pro-apoptotic to Non-Hodgkin's Lymphoma cells. The anti-HIV and antitumor peptides and truncated polypeptides of MAP30 are disclosed in US Patent 6,652,861. Table 4 in U.S. Pat. No. 6,652,861 lists the various MAP30 fragments and those with either a positive or negative antitumor effect. In particular, Type 1 Ribosomal Inhibiting Proteins (RIP) especially MAP30, are known to have robust and broad spectrum anticancer activity against a range of cancer cell types.

In particular, polypeptide A may be a Retrocyclin, polypeptide B may be MAP30 and polypeptide C may be a Dermaseptin. More in particular, polypeptide A may be Retrocyclin 101 (RC101) and polypeptide C may be Dermaseptin 1. A polypeptide comprising RC101, MAP30 and Dermaseptin 1 as polypeptide A, B and C respectively is termed RetroMAD1 in the present invention.

In particular, polypeptide A may comprise amino acid sequence with SEQ ID NO: 4, a fragment or variant thereof, polypeptide B may comprise amino acid sequence with SEQ ID NO:5, a fragment or variant thereof, and polypeptide C may comprise amino acid sequence with SEQ ID NO:6, a fragment or variant thereof.

The fusion protein according to any aspect of the present invention may further comprise at least one aptamer that may be linked to the peptide. For example, the aptamer may be at least one G-rich oligonucleotide. The peptide may be fused to an siRNA.

More in particular, the fusion protein according to any aspect of the present invention may comprise the amino acid sequence SEQ ID NO:1. The fusion protein or the basic unit of the fusion protein may have a molecular weight of about 30-50 kDa. In particular, the molecular weight of the fusion protein may be 31, 32, 33, 34, 35, 36, 37, 38, 39, 40, 41, 42, 43, 44, 36.5, 37, 37.5, 37.8, 38, 39, 40, 41, 42, 43, 44, 45, 46, 47, 48 or 49 kDa. The fusion protein may comprise repeats of the basic unit. A skilled person would understand that the weight of the fusion protein would be dependent on the multiples of the basic unit present in the protein. The nucleic acid coding for the fusion protein of SEQ ID NO:1 may be found in SEQ ID NO:2. The sequences are provided in Table 1 b below.

In particular, polypeptide B may be Type 1 RIP, or a fragment thereof, and polypeptide C may be Cationic AntiMicrobial Peptide, or a fragment thereof; and—may be a direct linkage or a linker peptide.

In particular, the fusion protein may comprise the formula XIV:

C-B-C

wherein, polypeptide C is CAP, an analogue, or a fragment thereof, polypeptide B is Type 1 RIP, or a fragment thereof, and—may be a direct linkage or a linker peptide.

In particular, the fusion protein may comprise the formula XX or XXI:

B-D or D-B

Respectively, wherein, polypeptide B is MAP30, an analogue, or a fragment thereof, polypeptide D is a synthetic anticancer sequence (KLAKLAK)2, or a fragment thereof, and—may be a direct linkage or a linker peptide.

TABLE 1b
Sequences of polypeptides and polynucleotides of the
present invention.

| SEQ | |
|---|---|
| ID | |
| NO. | Sequences |
| 1 | MKYLLPTAAAGLLLLAAQPAMAMGRICRCICGRGICRCICGVPGVGVPGVGGATGSDVNFDLSTATAKTY |
| | TKFIEDFRATLPFSHKVYDIPLLYSTISDSRRFILLDLTSYAYETISVAIDVTNVYVVAYRTRDVSYFFK |
| | ESPPEAYNILFKGTRKITLPYTGNYENLQTAAHKIRENIDLGLPALSSAITTLFYYNAQSAPSALLVLIQ |
| | TTAEAARFKYIERHVAKYVATNFKPNLAIISLENQWSALSKQIFLAQNQGGKFRNPVDLIKPTGERFQVT |
| | NVDSDVVKGNIKLLLNSRASTADENFITTMTLLGESVVEFPWALWKTMLKELGTMALHAGKAALGAAADT |
| | ISQGTQVPGVGVPGVGKLAAALEHHHHHH |
| 2 | atgaaatacctgctgccgaccgctgctgctggtctgctgctcctcgctgcccagccggcgatggccatgg |
| | ggcgtatttgccgttgcatttgcggccgtggcatttgccgctgcatctgtggcgtgccgggtgttggtgt |
| | tccgggtgtgggtggtgcgaccggatccgatgtgaactttgatctgagcaccgcgaccgcgaaaacctat |
| | accaaattcatcgaagattttcgtgcgaccctgccgtttagccataaagtgtatgatatcccgctgctgt |
| | atagcaccattagcgatagccgtcgttttattctgctggatctgaccagctatgcgtatgaaaccattag |
| | cgtggcgattgatgtgaccaacgtgtatgtggtggcgtatcgtacccgtgatgtgagctactttttcaaa |
| | gaaagcccgccggaagcgtacaacattctgtttaaaggcacccgtaaaattaccctgccgtataccggca |
| | actatgaaaacctgcagaccgcggcgcataaaattcgtgaaaacatcgatctgggcctgccggccctgag |
| | cagcgcgattaccaccctgttttattataacgcgcagagcgcgccgagcgcgctgctggtgctgattcag |
| | accaccgcggaagcggcgcgttttaaatatattgaacgccacgtggcgaaatatgtggcgaccaacttta |
| | aaccgaacctggccattattagcctggaaaaccagtggagcgccctgagcaaacaaatttttctggccca |
| | gaaccagggcggcaaatttcgtaatccggtggatctgattaaaccgaccggcgaacgttttcaggtgacc |
| | aatgtggatagcgatgtggtgaaaggcaacattaaactgctgctgaacagccgtgcgagcaccgcggatg |
| | aaaactttattaccaccatgaccctgctgggcgaaagcgtggtggaattcccgtgggcgctgtggaaaac |
| | catgctgaaagaactgggcaccatggcgctgcatgcgggtaaagcggcgctgggtgcggcagcggatacc |
| | attagccagggcacccaggttccgggcgtgggcgttccgggcgttggtaagcttgcggccgcactcgagc |
| | accaccaccaccaccactga |
| 3 | [VPXVG]ₙ |
| 4 | GRICRCICGRGICRCICG |
| 5 | GSDVNFDLSTATAKTYTKFIEDFRATLPFSHKVYDIPLLYSTISDSRRFILLDLTSYAYETISVAIDVTN |
| | VYVVAYRTRDVSYFFKESPPEAYNILFKGTRKITLPYTGNYENLQTAAHKIRENIDLGLPALSSAITTLF |
| | YYNAQSAPSALLVLIQTTAEAARFKYIERHVAKYVATNFKPNLAIISLENQWSALSKQIFLAQNQGGKFR |
| | NPVDLIKPTGERFQVTNVDSDVVKGNIKLLLNSRASTADENFITTMTLLGESVVEFPW |
| 6 | ALWKTMLKELGTMALHAGKAALGAAADTISQGTQ |

Modifications and changes may be made in the structure of the peptides of the present invention and DNA segments, which encode them and still obtain a functional molecule that encodes a protein or peptide with desirable characteristics. The amino acids changes may be achieved by changing the codons of the DNA sequence. For example, certain amino acids may be substituted for other amino acids in a protein structure without appreciable loss of interactive binding capacity with structures such as, for example, tumour or cancer cell-binding regions of fusion proteins. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. Various changes may be made in the peptide sequences of the disclosed compositions, or corresponding DNA sequences, which encode said proteins without appreciable loss of their biological utility or activity. Amino acid substitutions of the fusion protein according to the present invention may be possible without affecting the antitumour or anticancer effect of the isolated peptides of the invention, provided that the substitutions provide amino acids having sufficiently similar properties to the ones in the original sequences.

Examples of polypeptides according to any aspect of the present invention may be found in Table 1c and the DNA and protein sequences may be found in Tables 1d and 1e respectively.

TABLE 1c
Examples of fusion peptides

| | Example | | | |
|---|---|---|---|---|
| | Polypeptide A | Polypeptide B | Polypeptide C | Sequence listing |
| Fusion peptide | Defensin | RIP | CAP | — |
| RetroMAD1 | Retrocyclin 101 | MAP30 | Dermaseptin1 | SEQ ID NO: 1 |
| RetroGAD1 | Retrocyclin 101 | GAP31 | Dermaseptin1 | SEQ ID NO: 36 |
| Tamapal1 | Tachyplesin | MAP30 | Alloferon1 | SEQ ID NO: 34 |
| | Example | | | |
| | | | Polypeptide D | |
| | Polypeptide C | Polypeptide B | Pro-apoptotic | Sequence listing |
| Fusion peptide | CAP | RIP | peptide | — |
| K5 | Gaegurin 5 | MAP30 | (KLAKLAK)2 | SEQ ID NO: 35 |

TABLE 1d
DNA sequences of Amatilin, RetroGAD1, Tamapal1 and K5

| | SEQ | |
|---|---|---|
| Fusion | ID | |
| Protein | NO. | DNA Sequence |
| Amatilin | 37 | GGGCAGTGAGCGGAAGGCCCATGAGGCCAGTTAATTAAGAGGTACCGAATTCTCAT |
| | | TCGGTTTGTGTAGATTGAGAAGAGGTTTCTGTGCTCACGGTAGATGTAGATTCCCA |
| | | TCCATCCCAATCGGTAGATGTTCCAGATTCGTTCAGTGTTGTAGAAGAGTTTGGGT |
| | | CCCAGGTGTTGGTGTTCCAGGTGTTGGAGGTGCTACTGGTTCTGATGTTAACTTCG |
| | | ACTTGTCCACTGCTACTGCTAAGACTTACACTAAGTTCATCGAGGACTTCAGAGCT |
| | | ACTTTGCCATTCTCCCACAAGGTTTACGACATCCCTTTGTTGTACTCCACTATCTC |
| | | CGACTCCAGAAGATTCATCTTGTTGAACTTGACTTCCTACGCTTACGAGACTATCT |
| | | CCGTTGCTATCGACGTTACAAACGTTTACGTTGTTGCTTACAGAACTAGAGATGTT |
| | | TCCTACTTCTTCAAAGAGTCCCCACCAGAGGCTTACAACATCTTGTTCAAGGGTAC |
| | | TAGAAAGATCACTTTGCCATACACTGGTAACTACGAGAACTTGCAGACTGCTGCTC |
| | | ACAAGATCAGAGAGAACATCGACTTGGGTTTGCCAGCTTTGTCCTCCGCTATCACT |
| | | ACTTTGTTCTACTACAACGCTCAGTCCGCTCCATCCGCTTTGTTGGTTTTGATCCA |
| | | GACTACTGCTGAGGCTGCTAGATTCAAGTACATCGAGAGACACGTTGCTAAGTACG |
| | | TTGCTACAAACTTCAAGCCAAACTTGGCTATCATCTCCTTGGAGAACCAGTGGTCT |
| | | GCTTTGTCCAAGCAGATCTTCTTGGCTCAAAACCAGGGTGGTAAGTTCAGAAACCC |
| | | AGTCGACTTGATCAAGCCAACCGGTGAGAGATTCCAGGTTACTAATGTTGACTCCG |
| | | ACGTTGTTAAGGGTAACATCAAGTTGTTGTTGAACTCCAGAGCTTCCACTGCTGAC |
| | | GAGAACTTCATCACTACTATGACTTTGTTGGGTGAGTCCGTTGTTAACTCCTGTGC |
| | | TTCCAGATGTAAGGGTCACTGTAGAGCTAGAAGATGTGGTTACTACGTTTCCGTTC |
| | | TGTACAGAGGTAGATGTTACTGTAAATGTTTGAGATGTGTCCCCGGTGTTGGAGTC |
| | | CCTGGTGTCGGTGCGGCCGCGAGCTCATGGCGCGCCTAGGCCTTGACGGCCTTCCG |
| | | CCAATTCGC |
| RetroGAD1 | 38 | CGAATTGGCGGAAGGCCGTCAAGGCCACGTGTCTTGTCCAGGTACCGAATTCGGAA |
| | | TCTGTAGATGCATCTGCGGTAGAGGTATCTGCAGATGTATTTGTGGAAGAGTCCCA |
| | | GGTGTTGGTGTTCCAGGTGTTGGAGGTGCTACTGGTTCTGGTTTGGACACTGTTTC |
| | | ATTCTCCACTAAGGGTGCTACTTACATCACTTACGTTAACTTTTTGAACGAGTTGA |
| | | GAGTTAAGTTGAAGCCAGAGGGTAACTCCCACGGTATCCCTTTGTTGAGAAAGAAG |
| | | TGTGACGACCCAGGTAAGTGTTTCGTTTTGGTTGCTTTGTCCAACGACAACGGTCA |
| | | GTTGGCTGAGATTGCTATCGACGTTACTTCCGTTTACGTTGTTGGTTACCAGGTTA |
| | | GAAACAGATCCTACTTCTTCAAGGACGCTCCAGACGCTGCTTACGAAGGTTTGTTC |
| | | AAGAACACTATCAAGACTAGATTGCACTTCGGTGGTTCCTACCCATCTTTGGAAGG |
| | | TGAGAAGGCTTACAGAGAGACTACTGACTTGGGTATCGAGCCATTGAGAATCGGTA |
| | | TCAAGAAGTTGGACGAGAACGCTATCGACAACTACAAGCCAACTGAGATCGCTTCC |
| | | TCCTTGTTGGTTGTTATCCAGATGGTTTCCGAGGCTGCTAGATTCACTTTCATCGA |
| | | GAACCAGATCAGAAACAACTTCCAGCAGAGAATCAGACCAGCTAACAACACTATTT |
| | | CCTTGGAGAACAAGTGGGGTAAGTTGTCCTTCCAGATCAGAACATCCGGTGCTAAC |
| | | GGTATGTTCTCTGAGGCTGTTGAGTTGGAGAGAGCTAACGGTAAGAAGTACTACGT |
| | | TACTGCTGTTGACCAGGTTAAGCCAAAGATCGCTTTGTTGAAGTTCGTTGACAAGG |
| | | ACCCAAAGGGTTTGTGGTCCAAGATCAAAGAGGCTGCTAAGGCTGCTGGTAAGGCT |
| | | GCTTTGAATGCTGTTACTGGTTTGGTTAACCAGGGTGACCAACCATCTGTCCCTGG |
| | | TGTTGGAGTCCCTGGTGTCGGTGCGGCCGCGAGCTCTGGAGCACAAGACTGGCCTC |
| | | ATGGGCCTTCCGCTCACTGC |
| Tamapal1 | 39 | GGATCCGTTCCGGGTGTGGGTGTTCCGGGTGTTGGTAAATGGTGTTTCGTGTTTGT |
| | | TATCGCGGTATTTGTTATCGTCGTTGTCGTGTGCCAGGCGTTGGCGTTCCAGGCGT |
| | | GGGTGGTGCAACCGGTAGTGATGTTAATTTTGATCTGAGCACCGCAACCGCAAAAA |
| | | CCTATACCAAATTTATCGAAGATTTTCGTGCAACCCTGCCGTTTAGCCATAAAGTT |
| | | TATGATATTCCGCTGCTGTATAGCACCATTAGCGATAGCCGTCGTTTTATTCTGCT |
| | | GAATCTGACCAGCTATGCCTATGAAACCATTAGCGTTGCAATTGATGTGACCAATG |
| | | TTTATGTTGTTGCATATCGTACCCGTGATGTGAGCTATTTTTTCAAAGAAAGCCCT |
| | | CCGGAAGCCTATAACATTCTGTTTAAAGGCACCCGCAAAATCACCCTGCCGTATAC |
| | | CGGTAATTATGAAAATCTGCAGACCGCAGCACATAAAATTCGCGAAAATATTGATC |
| | | TGGGTCTGCCTGCACTGAGCAGCGCAATTACCACCCTGTTTTATTACAATGCACAG |
| | | AGCGCACCGAGCGCACTGCTGGTTCTGATTCAGACCACCGCAGAAGCAGCACGCTT |
| | | TAAATACATTGAACGTCATGTTGCCAAATACGTGGCCACCAACTTTAAACCGAATC |
| | | TGGCAATTATTAGCCTGGAAAATCAGTGGTCAGCACTGAGCAAACAAATTTTTCTG |
| | | GCACAGAATCAGGGTGGCAAATTTCGTAATCCGGTTGATCTGATTAAACCG |
| | | ACCGGTGAACGTTTTCAGGTTACCAATGTTGATAGTGATGTGGTGAAAGGCAACAT |
| | | TAAACTGCTGCTGAATAGCCGTGCAAGCACCGCAGATGAAAACTTTATTACCACCA |
| | | TGACCCTGCTGGGTGAAAGCGTTGTTAATGTTCCTGGTGTTGGCGTGCCTGGTGTT |
| | | GGTCATGGTGTTAGCGGTCATGGTCAGCATGGTGTTCATGGTTAAAAGCTT |
| K5 | 40 | GGATCCGTTCCGGGTGTGGGTGTTCCGGGTGTTGGCTTTCTGGGTGCACTGTTTAAA |
| | | GTTGCAAGCAAAGTTCTGCCGAGCGTTAAATGTGCAATTACCAAAAAATGTGTTCCT |
| | | GGCGTTGGTGTTCCAGGCGTGGGTGGTGCAACCGGTAGTGATGTTAATTTTGATCTG |
| | | AGCACCGCAACCGCAAAAACCTATACCAAATTTATCGAAGATTTTCGTGCAACCCTG |
| | | CCGTTTAGCCATAAAGTTTATGATATTCCGCTGCTGTATAGCACCATTAGCGATAGC |
| | | CGTCGTTTTATTCTGCTGAATCTGACCAGCTATGCCTATGAAACCATTAGCGTTGCA |
| | | ATTGATGTGACCAATGTTTATGTTGTTGCATATCGTACCCGTGATGTGAGCTATTTT |
| | | TTCAAAGAAAGCCCTCCGGAAGCCTATAACATTCTGTTTAAAGGCACCCGCAAAATC |
| | | ACCCTGCCGTATACCGGTAATTATGAAAATCTGCAGACCGCAGCACATAAAATTCGC |
| | | GAAAATATTGATCTGGGTCTGCCTGCACTGAGCAGCGCAATTACCACCCTGTTTTAT |
| | | TACAATGCACAGAGCGCACCGAGCGCACTGCTGGTTCTGATTCAGACCACCGCAGAA |
| | | GCAGCACGCTTTAAATACATTGAACGTCATGTTGCCAAATACGTGGCCACCAACTTT |
| | | AAACCGAATCTGGCAATTATTAGCCTGGAAAATCAGTGGTCAGCACTGAGCAAACAA |
| | | ATTTTTCTGGCACAGAATCAGGGTGGCAAATTTCGTAATCCGGTTGATCTGATTAAA |
| | | CCGACCGGTGAACGTTTTCAGGTTACCAATGTTGATAGTGATGTGGTGAAAGGCAAC |
| | | ATTAAACTGCTGCTGAATAGCCGTGCAAGCACCGCAGATGAAAACTTTATTACCACC |
| | | ATGACCCTGCTGGGTGAAAGCGTTGTTAATGTTCCAGGTGTTGGTGTGCCTGGTGTG |
| | | GGTAAACTGGCAAAACTGGCCAAAAAACTGGCTAAGCTGGCGAAATAAAAGCTT |

TABLE 1e
Polypeptide sequences of Amatilin, RetroGAD1, Tamapal1 and K5

| | SEQ | |
|---|---|---|
| Fusion | ID | |
| Protein | NO. | Protein Sequence |
| Amatilin | 28 | SFGLCRLRRGFCAHGRCRFPSIPIGRCSRFVQCCRRVWVPGVGVPGVGGATGSDVNF |
| | | DLSTATAKTYTKFIEDFRATLPFSHKVYDIPLLYSTISDSRRFILLNLTSYAYETIS |
| | | VAIDVTNVYVVAYRTRDVSYFFKESPPEAYNILFKGTRKITLPYTGNYENLQTAAHK |
| | | IRENIDLGLPALSSAITTLFYYNAQSAPSALLVLIQTTAEAARFKYIERHVAKYVAT |
| | | NFKPNLAIISLENQWSALSKQIFLAQNQGGKFRNPVDLIKPTGERFQVTNVDSDVVK |
| | | GNIKLLLNSRASTADENFITTMTLLGESVVNSCASRCKGHCRARRCGYYVSVLYRGR |
| | | CYCKCLRCVPGVGVPGVG |
| RetroGAD1 | 36 | GICRCIGRGICRCICGRVPGVGVPGVGGATGSGLDTVSFSTKGATYITYVNFLNELR |
| | | VKLKPEGNSHGIPLLRKKCDDPGKCFVLVALSNDNGQLAEIAIDVTSVYVVGYQVRN |
| | | RSYFFKDAPDAAYEGLFKNTIKTRLHFGGSYPSLEGEKAYRETTDLGIEPLRIGIKK |
| | | LDENAIDNYKPTEIASSLLVVIQMVSEAARFTFIENQIRNNFQQRIRPANNTISLEN |
| | | KWGKLSFQIRTSGANGMFSEAVELERANGKKYYVTAVDQVKPKIALLKFVDKDPKGL |
| | | WSKIKEAAKAAGKAALNAVTGLVNQGDQPSVPGVGVPGVG |
| Tamapal1 | 34 | VPGVGVPGVGKWCFRVCYRGICYRRCRVPGVGVPGVGGATGSDVNFDLSTATAKTYT |
| | | KFIEDFRATLPFSHKVYDIPLLYSTISDSRRFILLNLTSYAYETISVAIDVTNVYVV |
| | | AYRTRDVSYFFKESPPEAYNILFKGTRKITLPYTGNYENLQTAAHKIRENIDLGLPA |
| | | LSSAITTLFYYNAQSAPSALLVLIQTTAEAARFKYIERHVAKYVATNFKPNLAIISL |
| | | ENQWSALSKQIFLAQNQGGKFRNPVDLIKPTGERFQVTNVDSDVVKGNIKLLLNSRA |
| | | STADENFITTMTLLGESVVNVPGVGVPGVGHGVSGHGQHGVHG |
| K5 | 35 | VPGVGVPGVGFLPLLAGLAANFLPTIICFISYKCVPGVGVPGVGGATGSDVNFDLST |
| | | ATAKTYTKFIEDFRATLPFSHKVYDIPLLYSTISDSRRFILLNLTSYAYETISVAID |
| | | VTNVYVVAYRTRDVSYFFKESPPEAYNILFKGTRKITLPYTGNYENLQTAAHKIREN |
| | | IDLGLPALSSAITTLFYYNAQSAPSALLVLIQTTAEAARFKYIERHVAKYVATNFKP |
| | | NLAIISLENQWSALSKQIFLAQNQGGKFRNPVDLIKPTGERFQVTNVDSDVVKGNIK |
| | | LLLNSRASTADENFITTMTLLGESVVNVPGVGVPGVGKLAK KLAKLAK |

K5 and Tamapal1 have been shown to be capable of close to 99% inhibition of PI3K at low concentrations of 5 μg/ml, Both these peptide drugs could be a potential medical drug that functions by inhibiting a Phosphoinositide 3-kinase enzyme which may be part of this pathway and therefore, through inhibition, often results in tumour suppression. This high level of inhibition of PI3K at such low drug concentrations may also be very useful in combinatorial anticancer drug regimes that may involve other drugs outside of this class or also with drugs within this class that work primarily on other pathways. PI3K/AKT mediated signal transduction molecules and effects on gene expression that contribute to tumorigenesis may also be more selective, more effective and less toxic compared with existing methods. Current evidence has suggested that the PI3K/AKT pathway is visible target for novel antitherapeutic drugs of the present invention.

The fusion peptide according to any aspect of the present invention may be thermostable over a prolonged period of time even in the harshest conditions. Thermostability is an industrially significant attribute as cold-chain transportation will greatly increase logistics and handling costs that will contribute to the overall total cost of the medication. Also, if the drug is to be carried about to be consumed before meals, patient compliance will suffer if the requirement of low temperature storage in an absolute necessity. Thus, the ability to remain stable for 7 days even at elevated temperatures will allow for a wider usage and application of the therapeutic protein. The fusion protein may also be stable for short-term (about 15mins) exposure at 70° C.

In particular, there is provided that the fusion protein may be in a form of a medicament that may further comprise a pharmaceutically acceptable carrier, excipient, adjuvant, diluent and/or detergent. Such formulations therefore include, in addition to the fusion protein, a physiologically acceptable carrier or diluent, possibly in admixture with one or more other agents such as other antibodies or drugs, such as an antibiotic. Suitable carriers include, but are not limited to, physiological saline, phosphate buffered saline, phosphate buffered saline glucose and buffered saline. Alternatively, the fusion protein may be lyophilized (freeze dried) and reconstituted for use when needed by the addition of an aqueous buffered solution as described above. Routes of administration are routinely parenteral, including intravenous, intramuscular, subcutaneous and intraperitoneal injection or oral delivery. The administration can be systemic and/or local.

In particular, the medicament according to the present invention may comprise at least one fusion protein according to the present invention and a pharmaceutically acceptable carrier as above.

The medicament may be used for topical or parenteral administration, such as subcutaneous, intradermal, intraperitoneal, intravenous, intramuscular or oral administration. For this, the fusion protein may be dissolved or suspended in a pharmaceutically acceptable, preferably aqueous carrier. The medicament may contain excipients, such as buffers, binding agents, blasting agents, diluents, flavours, lubricants, etc. The composition can be used for a prevention, prophylaxis and/or therapy as an antitumour or anticancer agent.

In particular, the medicament according to any aspect of the present invention may be suitable for oral administration as the medicament may have a high resistance to pepsin & trypsin proteolysis. In particular, the presence of MAP30 surprisingly renders the fusion protein according to any aspect of the present invention stable for oral administration.

The medicament may further comprise a detergent. The detergent may be selected from the group consisting of sodium-ursodeoxycholate, sodium glycylursodeoxycholate, potassium-ursodeoxycholate, potassium glycylursodeoxycholate, ferrous-ursodeoxycholate, ferrous glycylursodeoxycholate, ammonium-ursodeoxycholate, ammonium glycylursodeoxycholate, sodium-tauroursodeoxycholate, sodium-N-methylglycylursodeoxycholate, potassium-tauroursodeoxycholate, potassium-N-methyglycylursodeoxy-cholate, ferrous-tauroursodeoxycholate, ferrous-N-methyglycylursodeoxycholate, ammonium-tauroursodeoxycholate, ammonium-N-methyglycylursodeoxycholate, sodium-N-methyltauroursodeoxycholate, potassium-N-methyltauroursodeoxycholate, ferrous-N-methyltauroursodeoxycholate, ammonium-N-methyltauroursodeoxycholate, sodium-cholate, sodium-deoxycholate, potassium-cholate, potassium-deoxycholate, ferrous-cholate, ferrous-deoxycholate, ammonium-cholate, ammonium-deoxycholate, sodium-chenodeoxycholate, sodium-glycylcholate, potassium-chenodeoxycholate, potassium-glycylcholate, ferrous-chenodeoxycholate, ferrous-glycylcholate, ammonium-chenodeoxycholate, ammonium-glycylcholate, sodium-taurocholate, sodium-N-methylglycylcholate, potassium-taurocholate, potassium-N-methylglycylcholate, ferrous-taurocholate, ferrous-N-methylglycylcholate, ammonium-taurocholate, ammonium-N-methylglycylcholate, sodium-N-methyltaurocholate, sodium-glycyldeoxycholate, potassium-N-methyltaurocholate, potassium-glycyldeoxycholate, ferrous-N-methyltaurocholate, ferrous-glycyldeoxycholate, ammonium-N-methyltaurocholate, ammonium-glycyldeoxycholate, sodium-taurodeoxycholate, sodium-N-methylglycyldeoxycholate, potassium-taurodeoxycholate, potassium-N-methylglycyldeoxycholate, ferrous-taurodeoxycholate, ferrous-N-methyl glycyldeoxycholate, ammonium-taurodeoxycholate, ammonium-N-methylglycyldeoxycholate, sodium-N-methyltaurodeoxycholate, sodum-N-methylglycylchenodeoxycholate, potassium-N-methyltaurodeoxycholate, potassium-N-methylglycylchenodeoxycholate, ferrous-N-methyltaurodeoxycholate, ferrous-N-methylglycylchenodeoxycholate, ammonium-N-methyltaurodeoxycholate, ammonium-N-methylglycylchenodeoxycholate, sodium-N-methyltaurochenodeoxycholate, potassium-N-methyltaurochenodeoxycholate, ferrous-N-methyltaurochenodeoxycholate, ammonium-N-methyltaurochenodeoxycholate, ethyl esters of ursodeoxycholate, propyl esters of ursodeoxycholate, sodium-glycylchenodeoxycholate, potassium-glycylchenodeoxycholate, ferrous-glycylchenodeoxycholate, ammonium-glycylchenodeoxycholate, sodium-taurochenodeoxycholate, potassium-taurochenodeoxycholate, ferrous-taurochenodeoxycholate, ammonium-taurochenodeoxycholate, sodium deoxycholate and the like. In particular, the detergent may be sodium deoxycholate that allows for oral administration as it may result in the fusion protein not being digested in the gastrointestinal tract when consumed. This is a convenient mode of administration.

The detergent may be present at a concentration of 0.003-5% by weight. In particular, the concentration may be 0.01-4.5 wt %, 0.05-4 wt %, 0.1-3.5 wt %, 0.5-2 wt %, 1-1.5 wt %, and the like. In particular, the concentration of the detergent may be about 0.05 wt %.

The medicament according to the present invention may comprise at least one of the fusion proteins of the present invention and may be administered to a patient having tumour and/or a cancerous growth.

The dosage of the ligand according to the present invention to be administered to a patient having tumour or cancer may vary with the precise nature of the condition being treated and the recipient of the treatment. The dose will generally be in the range of about 0.005 to about 1000 mg for an adult patient, usually administered daily for a period between 1 day to 2 years. In particular, the daily dose may be 0.5 to 100 mg per day. In particular the daily dose may be about 0.8, 1, 1.2, 1.5, 2, 2.5, 3.2, 4, 4.5, 5, 10, 15, 20, 30, 45, 50, 75, 80, 90, 95 mg per day. The dosage may be applied in such a manner that the ligand may be present in the medicament in concentrations that provide in vivo concentrations of said ligand in a patient to be treated of between 0.001 mg/kg/day and 5 mg/kg/day. In one embodiment, the medicament, the peptide or ligand according to the invention is present in an amount to achieve a concentration in vivo of 1 μg/ml or above with a maximum concentration of 100 μg/ml. the dosage regime may be varied depending on the results on the patient.

In one example, the patient may be given at least one medicament comprising at least a first fusion protein for a period of 1 month to 2 years. The first fusion protein may be taken for a period of 1, 2, 3, 4, 5, 6, 7, 8, 9, 10, 11, 12, 13, 14, 15 months. Once the first fusion protein appears less effective or not as effective as before on treating the cancer and/or tumour, a second fusion protein according to any aspect of the present invention may be administered to the patient. The second fusion protein may be different from the first fusion protein. Once the second fusion protein appears less effective or not as effective as before on treating the cancer and/or tumour, a third, fourth fifth, sixth etc. fusion protein according to any aspect of the present invention may be administered to the patient each protein may be different from the earlier protein. This dosage regime may prevent resistant cancer cells from proliferating thus providing an effective and efficient cancer therapy.

The medicament of the present invention can further contain at least one host defence molecule, such as lysozyme, lactoferrin and/or Reverse-Transcriptase inhibitor.

The fusion protein according to any aspect of the present invention may be capable of maintaining its form in the digestive tract without fragmentation or enzymatic digestion. In one example, the fusion protein may be in a liquid form. In particular, the fusion protein may be ingested, as a drink diluted with water, or the like, and the retention time in either stomach or duodenum is only a matter of minutes allowing the protein to reach its target point without being digested.

The fusion protein and medicament according to any aspect of the present invention may be used for treatment and/or prevention of cancer. The cancer may be a microbe induced cancer. Microbes which induce cancer may include by are not limited to bacteria, viruses and the like. These microbes may be classified as Class A, B or C microbes. Class A microbes induce cancers including lymphomas by targeting immunocytes leading to immunosuppression. This immunosuppression also contributes to the cancer-inducing effects of class B microbes, which include local effects on parenchymal cells and induction of host responses. Class B microbes may induce the most commonly recognized microbe-associated cancers. Class C microbes are a postulated class in which a microbe produces local effects on epithelial tissues that change the regulation of a systemic operator (e.g., a hormone) that promotes cancer at a distant site.

Non-limiting examples of class A agents include human T-cell lymphotrophic virus type 1, which may promote adult T-cell leukemia/lymphoma, and HIV, which may promote lymphoma development and, through immunosuppression, other microbe-induced malignancies including human herpesvirus-8 induced Kaposi's sarcoma and HPV-induced anogenital cancers.

The numerous examples of class B processes include carcinomas due to the hepatitis viruses, H. pylori and the like. Class C agents, with local effects that can lead to either distant or other local effects may include H. pylori—induced development of atrophic gastritis which could lead to repopulation with microbiota that are toxic to gastric tissue and directly oncogenic, or microbiome-induced disturbances in hormonal regulation could lead to cancers distant from the locus of the change.

In particular, cancer bacteria may include Salmonella typhi which may be associated with gallbladder cancer, Streptococcus bovis which may be associated with colorectal cancer, Chlamydia pneumoniae which may be associated with lung cancer, Mycoplasma which may be associated with formation of different types of cancer, Helicobacter pylori which may be linked to stomach cancer, gastric cancer, MALT lymphoma, esophageal cancer and the like.

Cancer viruses may be known as oncoviruses that may include DNA viruses and/or RNA viruses. The DNA viruses may include but are not limited by Human papilloma virus (HPV) which may cause transformation in cells through interfering with tumor suppressor proteins such as p53 and thus causing cancers such as cancers of cervix, anus, penis, vulva/vagina, and some cancers of the head and neck. Other DNA viruses include Kaposi's sarcoma-associated herpesvirus (KSHV or HHV-8) which may be associated with Kaposi's sarcoma, a type of skin cancer, Epstein-Barr virus (EBV or HHV-4) which may be associated with Burkitt's lymphoma, Hodgkin's lymphoma, post-transplantation lymphoproliferative disease, Nasopharyngeal carcinoma and the like, Merkel cell polyomavirus—a polyoma virus—may be associated with the development of Merkel cell carcinoma, Human cytomegalovirus (CMV or HHV-5) which may be associated with mucoepidermoid carcinoma and possibly other malignancies, HSV-1 or HSV-2 which may be associated with oral cancers, SV40 which may be associated to Non-Hodgkin's Lymphoma and the like.

RNA viruses include but are not limited to hepatitis A, B and C viruses which are associated with Hepatocellular carcinoma (liver cancer), human T-lymphotropic virus (HTLV-1) which is associated with Tropical spastic paraparesis and adult T-cell leukemia and the like.

The cancer may be selected from the group consisting of Non-Hodgkin's Lymphoma, brain, lung, colon, epidermoid, squamous cell, bladder, gastric, pancreatic, breast, head, neck, renal, kidney, liver, ovarian, prostate, colorectal, uterine, rectal, oesophageal, testicular, gynecological, thyroid cancer, melanoma, hematologic malignancies such as acute myelogenous leukemia, multiple myeloma, chronic myelogneous leukemia, myeloid cell leukemia, glioma, pontine glioblastoma, Kaposi's sarcoma, and any other type of solid or liquid cancer.

The fusion protein may be pegylated to aid in the medicament being suitable for oral delivery. In particular, the fusion protein may be pegylated with any PEG known in the art. The PEG may be selected from the group consisting of but not limited to PEG200, 300, 400, 500, 600, 700, 800, 900, 1000, 1100, 1200, 1300, 1400, 1500, 1600, 1700,1800,1900, 2000, 2100, 2200, 2300, 2400, 2500, 2600, 2700, 2800, 3000, 3250, 3350, 3500, 3750, 4000, 4250, 4500, 4750, 5000, 5500, 6000, 6500, 7000, 7500, 8000 and the like.

In one aspect of the present invention there is provided a method of treating a tumour or cancer in a subject in need thereof, comprising administering to the subject an effective amount of the fusion protein or the medicament according to any aspect of the present invention.

In yet another aspect of the present invention there is provided the fusion protein or the medicament according to any aspect of the present invention for treating a tumour or cancer in a subject in need thereof.

A person skilled in the art will appreciate that the present invention may be practised without undue experimentation according to the method given herein. The methods, techniques and chemicals are as described in the references given or from protocols in standard biotechnology and molecular biology text books.

The fusion protein and/or pharmaceutical composition according to any aspect of the present invention may result in no or substantially no toxic side effects when taken by the subject.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Sambrook and Green, Molecular Cloning: A Laboratory Manual, Cold Springs Harbor Laboratory (Fourth Edition), New York (2012).

### Example 1

### Construction and Design of Expression Vector

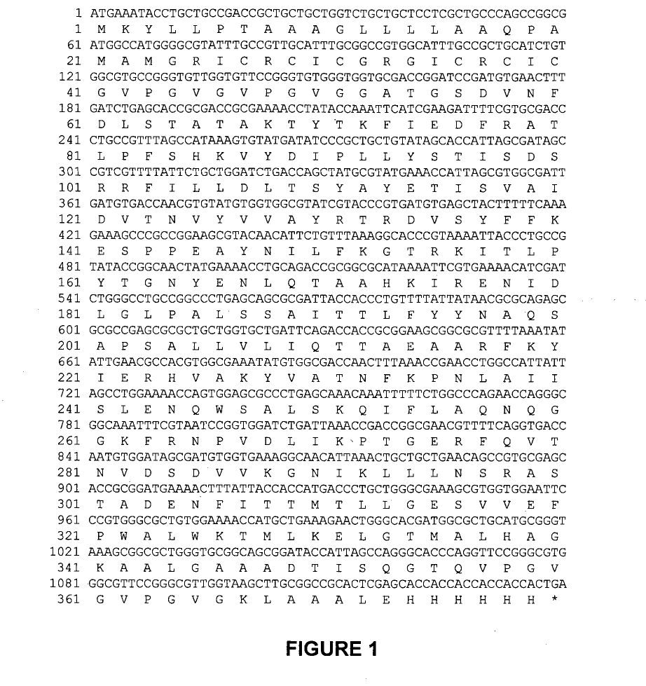

The gene encoding RetroMAD1 A-B-C with SEQ ID NO:1 was synthesized and cloned into backbone of vector pGA4 at the KpnI/SacI site by contract service (GeneArt AG, Germany). The expected product size was 1140 bp, which encoded a 379 amino acid and an expected size of 41.2 kDa. The polynucleotide sequence and the translated polypeptide sequence are shown in FIG. 1 from PCT. The gene was sub-cloned into a pET expression vector (Novagen), pET-26(b) at the NcoI/HindIII sites. Kanamycin was used as a marker for selection and maintenance of culture purposes. This vector was inducible under the addition of isopropyl-beta-D-thiogalactopyranoside (IPTG). The plasmid, pRMD1 was then transformed into BL21(DE23) cells (Novagen) and plated on a selective media with Kanamycin.

### Expression of RetroMAD1 from E. coli

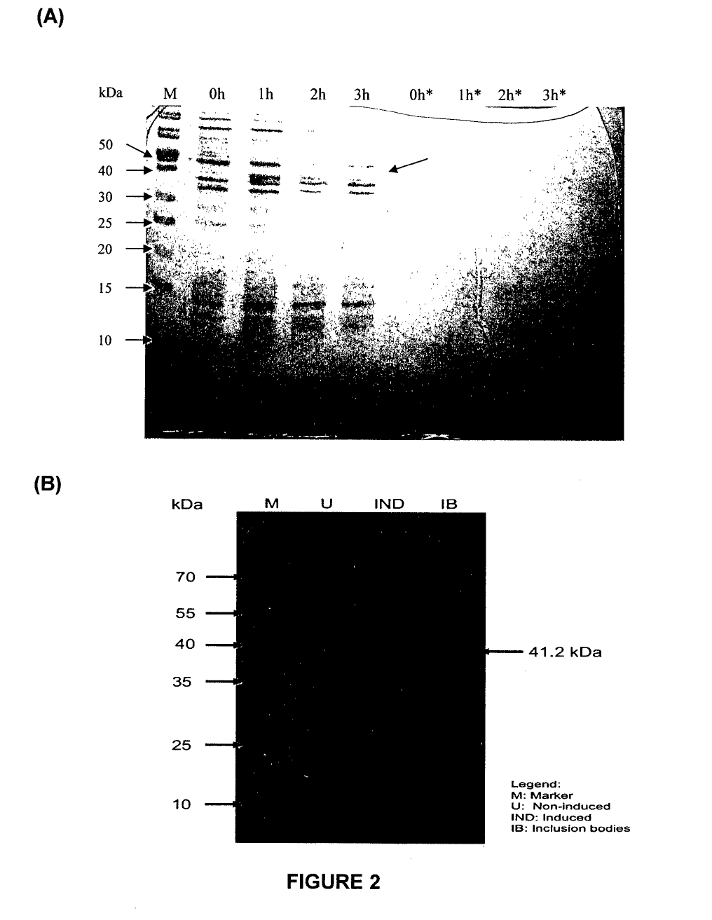

One recombinant clone was grown in 10 ml of LB Bertani (DIFCO) medium, supplemented with 30 μg/ml kanamycin, at 37° C. overnight. This culture was used to inoculate 100 ml of LB Bertani supplemented with 30 μg/m1 kanamycin and grown at 37° C. until the optical reading was 0.4-0.6 at 600 nm. IPTG was added at 1.0 mM final concentration. The growth period continued for 3 hours. An SDS-PAGE analysis of the fraction of RetroMAD1 in cells extracted in electrophoresis loading buffer showed that a protein had a molecular mass of about 37.5 kDa, the expected molecular size of RetroMAD1 was produced in the induced cells only (FIG. 2A). Further solubility analysis by SDS-PAGE revealed that RetroMAD1 was found in the pellet fraction and not in the supernatant fraction of the E. coli indicating that the protein was expressed and produced as inclusion bodies as shown in FIG. 2B.

### Isolation and Purification of RetroMAD1

Cells from 100 ml of induced culture were harvested by centrifugation for 10 min at 5000×g at 15° C. The cells were suspended in a lysis buffer containing 20 mM Tris-HCl (pH 7.5), 10 mM EDTA and 1% Triton-X 100. Cells were disrupted by sonication. The insoluble fraction was isolated from the soluble fraction by centrifugation at 8,000×g for 20 min. The supernatant was discarded and the pellet was further washed by repeating the same step. The pellet was further washed twice with RO water by resuspension via sonication and separation by centrifugation.

### Solubilization of RetroMAD1

The insoluble material was dissolved and sonicated in 10 ml of 5-8 Urea or 6M Guanidine Hydrochloride and supplemented with 2-5% of Sodium-lauryl sarcosine and 100 mM β-mercaptoethanol. The solubilisation was carried out overnight. The solubilised protein was separated from the bacterial cell wall by centrifugation at 8,000×g for 20 minutes.

### Refolding of RetroMAD1

Renaturation of the protein was carried out by using dialysis. The protein (10 ml) was dialysed in a 15 kDa molecular weight cut-off dialysis membrane (Spectra/Por Lab). The protein was dialysed in 5L of RO water with the pH of 11.0 adjusted by NaOH. Incubation was done at room temperature for 15-20 hours. The refolded protein was transferred to a 50 ml tube and centrifuged at 8,000×g to separate any insoluble material. Renatured protein was stored at −20° C. until further use. The bioactivity of RetroMAD1 in the following examples is proof of successful refolding of the protein.

### Example 2

### Preparation of Peripheral Blood Mononuclear Cells (PBMCs)

PBMC were isolated and blood samples collected into a 10 ml ethylenediaminetetraacetic acid (EDTA)-coated tube by density gradient centrifugation method. It was diluted at the ratio of 1:3 with RPMI-1640 (HyClone), layered onto Lymphoprep (Axis-Shield) and centrifuged at 2000 rpm for 30 minutes. During centrifugation, the PBMCs moved from the plasma and were suspended in density gradient. The PBMCs was washed twice with RPMI-1640 and subsequently were with RPMI-1640 medium. Cell viability was determined by tryphan blue exclusion method. The PBMC cell density used in this study was 1×10⁶ cells/well of the 96-well tissue culture plate. PBMC of Non-Hodgkins' Lymphoma patient was incubated with twelve different concentrations of RetroMAD1 for a period of 72 hours. Cell viability was found to decrease as the range of drug concentration increases from 0.05 μg/ml to 3.13 μg/ml. Cells are found to be most viable at the drug concentration range between 6.25 μg/ml to 50 μg/ml (Table 2).

TABLE 2
Simultaneous treatment with twelve dilutions of RetroMAD1
and its respective percentage of cell viability.

| Concentration (μg/ml) | Cell count | Cell viability (%) |
|---|---|---|
| 0.00 | 475366 | 100.0 |
| 0.05 | 194738 | 41.08 |
| 0.10 | 233484 | 49.26 |
| 0.20 | 195111 | 41.16 |
| 0.39 | 212544 | 44.84 |
| 0.78 | 284545 | 60.03 |
| 1.56 | 311700 | 65.75 |
| 3.13 | 382244 | 80.64 |
| 6.25 | 298088 | 62.89 |
| 12.50 | 325501 | 68.67 |
| 25.0 | 329405 | 69.49 |
| 50.0 | 460283 | 97.10 |
| 100.0 | 423347 | 89.31 |

### In vitro Virus Inhibition Assay

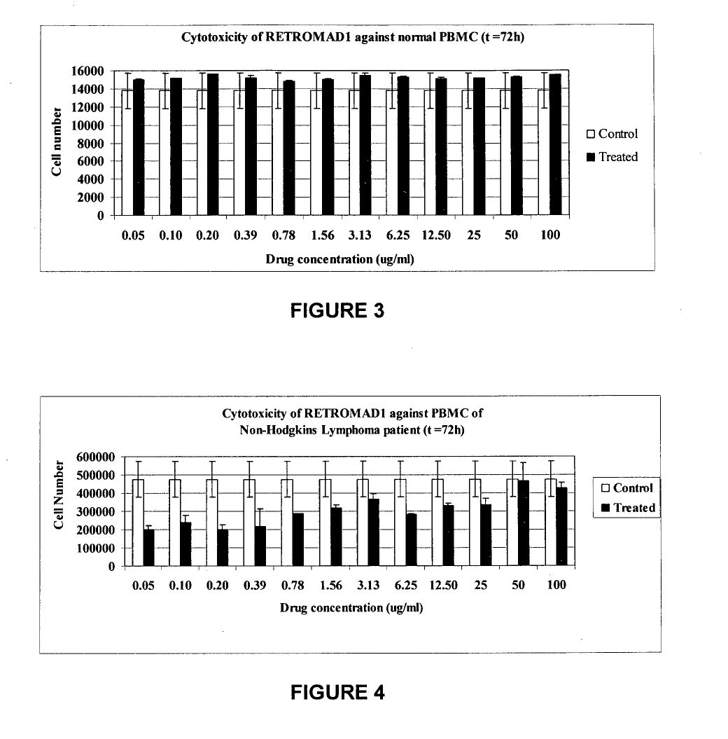

The in vitro virus inhibition assay of RetroMAD1 was carried out in triplicates of wells of a 96 wells plate with the cells were treated simultaneously. Twelve dilutions of RetroMAD1 (concentration of stock: 100 μg/ml) were used to treat both normal and infected PBMC simultaneously and the plate was incubated for 72 hours. At post-72 hours incubation time, the culture was collected. The results are shown in FIGS. 3 and 4. RetroMAD1 was shown not to affect the viability of PBMC isolated from normal donor of the same gender and similar age group (FIG. 4). Therefore, it appears that RetroMAD1 is able to selectively cause the decline of anomalous PBMCs due to its reported ability to target cells where the ultrastructure were altered by viral infection or cancer or both. This is because the MAP30 part of RetroMAD1 has been shown to display 10× more selective toxicity to specific leukemia cells compared to normal PBMCs (Lee-Huang, S. et al.,2000).

The selective cytotoxicity observed in PBMCs isolated from NHL patients may also have been due to the ability of cationic antimicrobial peptides to form ion channels through membrane bilayers that could selectively target the NHL PBMC that had increased permeability due to cancer related cell surface abnormalities. Increased permeability of cancer cells is has been shown by increased uptake of 67 [Ga] citrate. Atomic Force Microscopy (AFM) has also shown major differences in cell surface morphology between normal and cancer cells also providing further evidence to confirm the difference in uptake between cancer and normal cells.

### Example 3

### Teratogenicity Studies

Thirty, Day 1 pregnant Sprague Dawley (SD) adult female rats were randomly divided into 3 groups and each group fed orally with (a) sterile distilled water (Control) (1 ml/kg bodyweight, 0.2 ml/200 g rat); (b) 5 mg/kg of RetroMAD1 prepared in normal saline (low dose) and (c) 10 mg/kg of RetroMAD1 prepared in normal saline (high dose). The above mentioned regime was carried out for the adult female rats from day 1 pregnancy to day 20 and continued for 21 days post-delivery.

There are no signs of maternal toxicity or embryogenicity at 10 mg drug/kg body weight of pregnant rats treated from day 1 to day 20. There are no external fetal abnormalities, no growth delay, and no fetal death. The dam's (mother) weight gain after dosing, low and high dose of drug (gestational days 1 to 20) were comparable to normal control group. None of the pregnant rats delivered prematurely. The duration of gestation was unaffected by RetroMAD1.

There was no difference observed in dam-pup interactions between the drug-treated groups and normal control group. Each dam was able to nurse, and each pup was able to suckle. There were no observed differences between the groups as to when the offspring began to grow hair, crawl, sit, or wean. Prenatal drug treatment does not significantly change maternal behaviour toward pups because the frequency of active and passive nursing and pup grooming remained comparable in the drug-treated groups and normal control group. The frequency of dam-related behaviours (self-grooming, eating and drinking, and wandering active or passive) in drug-treated dams was also comparable to normal control dams. The frequency of nest-building activity was similar in drug-treated mother and normal control mothers.

Dams treated with the drug proceeded normally post-delivery and was terminated on day 21. Drug-treated dams did not present any abnormal type of behavior and they could not be physically distinguished from normal control dams, throughout gestation. The overall appearance of the normal control and drug-treated offspring was healthy and no differences were noted in litter size and offspring. No differences were found in the gestation length of control and drug-treated groups, nor were differences observed in litter size or number of stillborn pups.

No external signs of malformation were detected in the pups. There was no mortality in pups between drug treated groups compared with normal control group. From PND 1 to PND 21 there were no differences between the drug-treated group and the control group in the mean pups' body weight. There were no differences between the maternal groups in the number of pups per litter. The groups did not differ in the number of stillbirths, the viability index, and the lactation index. There were no significant differences in body weight, length or rate of growth of the offspring between the drug-treated groups and normal control group (PND 1 to 21) indicating normal postnatal growth unaffected by the prenatal drug treatment.

Physical development markers showed no drug treatment effect. All groups exhibited incisor eruptions (postnatal day 9) and eye openings (postnatal day 14). Pups of the drug-treated groups did not differ from their normal control counterparts in the time of pinna detachment. By PND 4, all of pups in all groups had their pinna detached. Pups born to drug-treated mothers did not differ from normal control pups in the time of incisor eruption and in the time of eye opening. The locomotors activity of the pups in drug-treated groups was comparable to that of normal control group.

TABLE 3
Comparison of physical and behavioral characteristics of rats
(dams and offspring) according to treatment group.

| | | Low | |
|---|---|---|---|
| Group | Control | dose | High dose |
| Premature delivery | none | none | none |
| Gestation period (no. | 20 | 20 | 20 |
| days) | | | |
| Foetal abnormalities | none | none | none |
| Foetal growth delay | none | none | none |
| Foetal death | none | none | none |
| Dam-pup interaction | normal | normal | normal |
| Dam-related behaviour | normal | normal | normal |
| Pup behavior | normal | normal | normal |
| Mean body weight | comparable in all groups | | |
| Postnatal growth | normal | normal | normal |
| Locomotion activity | normal | normal | normal |
| Pup Incisor eruptions | PND9 | PND9 | PND9 |
| Pup eye opening | PND4 | PND4 | PND4 |
| Pinna detachment | PND4 | PND4 | PND4 |

Note:
PND = postnatal day.

### Example 4

### Evidence of Bioavailability

The pharmacokinetic data of RetroMAD1 was derived in 6-8 weeks female ICR mice. Mice (48) were administered with single dose of RetroMAD1 of 70 ul per mouse which is a 50× dose of 0.2 mg/kg body weight given orally for ten days. Each day blood samples were drawn from the heart of three mice and one control. For the first day after the feed, the blood was collected after 30 min, 1 hour, 2 hour, 4 hour, 8 hour and 12 hours after oral administration and for the following days (up to day 10) the blood was collected just 30 min after administration. Each time point consisted of 3 mice fed orally with the drug and one control given PBS. Plasma concentration of RetroMAD1 was determined using an in house developed ELISA.

### ELISA for Detecting RetroMAD1 in Mice Sera: In house Capture ELISA with Anti Human-IgG-HRP

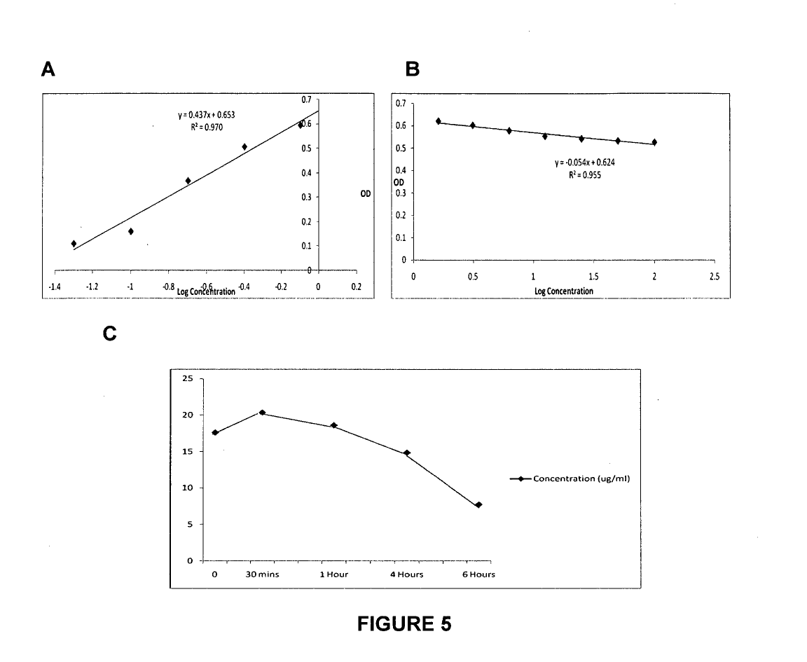

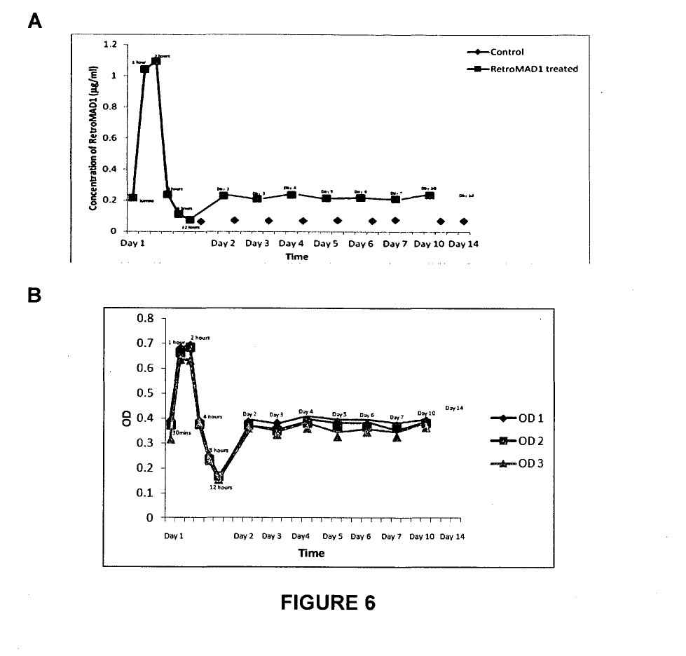

To prepare the capture antibody a cat was fed daily with RetroMAD1 and after 6 months blood harvested and serum extracted. This serum was used as the capture antibody. 100 ul/well of this polyclonal cat anti-RetroMAD1 antibody diluted 1:80 in coating buffer (0.2 M sodium carbonate-bicarbonate, ph 9.6) was adsorbed onto 96-well polystyrene ELISA plates. The plates were incubated at 4° C. overnight. Plates were washed three times with 0.05% Tween-20 in PBS 1×. 100 ul/well of mice serum diluted 1:2 in 0.05% BSA in PBS and were added to the wells. After incubation at 37° C. for 1 h, plates were washed similarly and 100 ul of anti RetroMAD1 positive human serum diluted 1:2000 in 0.05% BSA in PBS, was added. This antibody was obtained from the Department of Medical Microbiology, Faculty of Medicine, University Malaya, Malaysia. After incubation at 37° C. for 1 h, plates were washed and 100 ul/well Rabbit anti-human IgG HRP conjugate diluted 1:6000 in 0.05% BSA in PBS, was added. After incubation at 37° C. for 1 h in the dark, plates were washed and 100 ul/well of OPD added to each well. Plates were incubated in the dark for 30 min at room temperature and reaction stopped with 50 ul/well of 4N H2SO4. Optical densities (OD) were measured at 490 nm and 600 nm as background. All OD readings were then converted to Log values to obtain concentrations in ug/ml and the standard curves provided in FIG. 5. The results of the tests are provided in Table 4 and FIGS. 6A and B. The PK/PD data showed that RetroMAD1 was detected in the serum as early as 30 min post feeding at about 0.2 μg/ml that reached a maximum at 1-2hrs at 1-1.1 μg/ml before falling again to about 0.2 μg/ml at 4 hrs. By 12 hrs post feeding, levels were almost similar to the unfed controls indicating that the protein had been completely metabolized. Subsequent daily sampling 30 min post feeding indicated levels around 0.2 μg/ml. These data suggest bioavailability of the drug.

TABLE 4
Results of bioavailability test

| Day | Time | | OD 1 | OD 2 | OD 3 | Average | y = 0.437x + 0.6533 | |
|---|---|---|---|---|---|---|---|---|
| Day | 30 | mins | 0.391743 | 0.374396 | 0.317144 | 0.361094333 | −0.668662853 | 0.214455479 |
| 1 | 1 | hr | 0.683215 | 0.66296 | 0.637182 | 0.661119 | 0.017892449 | 1.042059336 |
| | 2 | hr | 0.632854 | 0.685153 | 0.692951 | 0.670319333 | 0.038945843 | 1.093819957 |
| | 4 | hr | 0.375195 | 0.376294 | 0.391285 | 0.380924667 | −0.623284516 | 0.238075927 |
| | 8 | hr | 0.234143 | 0.247498 | 0.229154 | 0.236931667 | −0.952787948 | 0.111483874 |
| | 12 | hr | 0.16735 | 0.154429 | 0.16771 | 0.163163 | −1.121594966 | 0.075579677 |
| | Control | | 0.132178 | | | 0.132178 | −1.192498856 | 0.064194991 |
| Day | 30 | mins | 0.387735 | 0.359613 | 0.372947 | 0.373431667 | −0.640430969 | 0.228859546 |
| 2 | Control | | 0.152749 | | | 0.152749 | −1.145425629 | 0.07154419 |
| Day | 30 | mins | 0.334864 | 0.352838 | 0.382846 | 0.356849333 | −0.678376812 | 0.209711955 |
| 3 | Control | | 0.149021 | | | 0.149021 | −1.153956522 | 0.070152553 |
| Day | 30 | mins | 0.360735 | 0.382153 | 0.395173 | 0.379353667 | −0.626879481 | 0.236113337 |
| 4 | Control | | 0.148574 | | | 0.148574 | −1.154979405 | 0.069987518 |
| Day | 30 | mins | 0.386559 | 0.367518 | 0.327878 | 0.360651667 | −0.66967582 | 0.213955857 |
| 5 | Control | | 0.156574 | | | 0.156574 | −1.136672769 | 0.073000735 |
| Day | 30 | mins | 0.347217 | 0.369173 | 0.3797746 | 0.3653882 | −0.658837071 | 0.219362774 |

| | | | | | |
|---|---|---|---|---|---|
| 6 | Control | 0.14443 | 0.14443 | −1.164462243 | 0.068475901 |

### Example 5

### Further Evidence of Bioavailability

In Guinea Pig PK/PD study, prior to experiment with RetroMAD1, the Guinea Pigs were starved overnight. The guinea pigs were then fed orally with RetroMAD1 according to their body weight; guinea pigs weighing from 380-430 g were fed orally with 250 μl of 3.5 mg/ml RetroMAD1, while guinea pigs weighing from 440-520 g were fed with 300 μl of 3.5 mg/ml RetroMAD1, and the controls were fed with water. At each time point, 3 guinea pigs were fed orally with RetroMAD1 and 3 guinea pig as control were fed with water. Before bleeding, the guinea pigs were given anesthesia (Ketamine and Xylazine) intramuscularly; the sedative dose was calculated using the following formula,

Ketamine=(45×body weight of the guinea pig)/(Concentration of Ketamine, 100 mg/ml)

Xylazine=(4.5×body weight of the guinea pig)/(Concentration of Xylazine, 20 mg/ml)

The guinea pigs were bled at 0, 30 mins, 1, 4 and 6 hours after feeding, blood samples were drawn from the heart. Serum of both control (untreated) and RetroMAD1-treated mice was collected for capture ELISA assay to determine the concentration of RetroMAD1 in the blood system.

Guinea pig organs were harvested. The organs are stomach, small intestine, liver, kidney.

Organs

| | |
|---|---|
| Stomach, Small | Collected into 15 ml of PBS for capture ELISA assay |
| Intestine | |
| Kidney, Liver | Snap freeze with liquid nitrogen |
| Kidney, Liver | Collected into distilled water and homogenized |
| Kidney, Liver | Collected into formalin for histology study |

### Capture ELISA

Capture ELISA using rabbit serum and anti-RetroMAD1 positive human serum was used to determined concentration of RetroMAD1 in the blood, stomach and small intestine.

In this capture ELISA, 100 μl of 1:1000 rabbit serum containing polyclonal rabbit anti-RetroMAD1 antibody was coated onto each well. The plates were incubated at 4° C. overnight. Plates were washed six times with 0.05% Tween-20 in PBS. The plates were then blocked with blocking buffer (10% BSA in PBS), 200 μl of blocking buffer was added to each well and was incubated for 2 hours at 37° C. Plates were then washed six times with 0.05% Tween-20 in PBS. 100 pl of guinea pig sample(serum/small intestine supernatant/stomach supernatant) were added to each wells and incubated at 37° C. for 1 hour, plates were then washed. 100 ul of 1:2500 anti-RetroMAD1 positive human serum. After incubation at 37° C. for 1 hour, the plates were washed. 100 μl 1:4800 Rabbit anti-human IgG HRP was added and incubated at 37° C. for 1 hour in the dark, plates were then washed. 100 ul of OPD added to each well and the plates were incubated in the dark for 30 min at room temperature. Finally, 50 ul of 4N H2SO4 was added to each well to stop the reaction. Optical densities (OD) were measured at 490 nm and 600 nm as background.

A standard curve was first generated by doing the capture ELISA as described above with RetroMAD1 of 1/2 dilution, the concentrations of RetroMAD1 are 100, 50, 25, 12.5, 6.25, 3.125, 1.6, 0.8, 0.4, 0.2 and 0.1 μg/ml. The equation of the standard curve was used to determine concentration of RetroMAD1 in serum, stomach and small intestine.

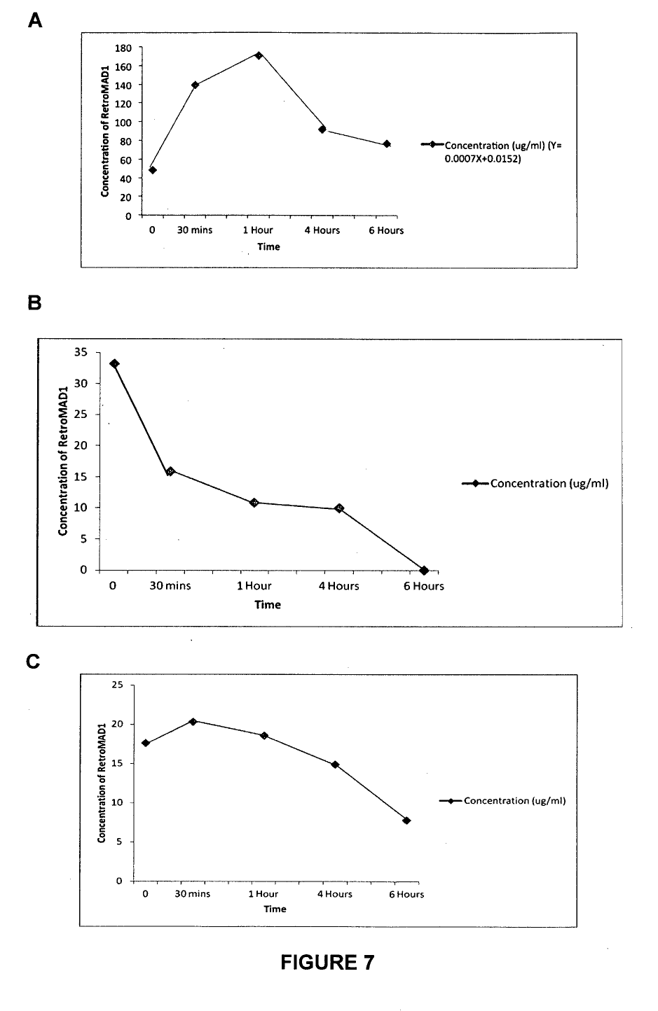

The PK/PD data for guinea pig serum is shown in Table 5A and FIG. 7A, result showed that RetroMAD1 was detected in the serum as early as 30 min post feeding at about 130 μg/ml that reached a maximum at 1hour at 170 μg/ml before falling again to about 90 μg/ml at 4 hours and 76 μg/ml at 6 hours. At 6 hours, the concentration of RetroMAD1 is more than the unfed controls indicating that the protein is not fully metabolized yet.

Data for guinea pig small intestine supernatant is shown in Table 5B and FIG. 7B. Result showed that highest concentration of RetroMAD1 was detected at 30 minutes at about 16 μg/ml. The concentration of RetroMAD1 then starts to fall to about 11 μg/ml at 1 hour, 9 μg/ml at 4 hours. And is then release from the small intestine at 6 hours where no RetroMAD1 was detected.

TABLE 5A
Results of bioavailability test in serum of guinea pig

| | | | | | Concentration |
|---|---|---|---|---|---|
| | | | | | (ug/ml) (Y = |
| | | | | | 0.0007X + |
| Time | OD1 | OD2 | OD3 | Average | 0.0152) |
| 0 | 0.047875 | 0.048515 | 0.050432 | 0.048941 | 48.2 |
| 30 mins | 0.118283 | 0.103765 | 0.115757 | 0.112602 | 139.15 |
| Control | 0.042267 | 0.042888 | 0.031889 | 0.039015 | 34.02 |
| 1 Hour | 0.132425 | 0.138091 | 0.132801 | 0.134439 | 170.34 |
| Control | 0.033272 | 0.043224 | 0.0398 | 0.038765 | 33.66 |
| 4 Hours | 0.089203 | 0.066944 | 0.082124 | 0.079424 | 91.75 |
| Control | 0.034081 | 0.031897 | 0.037074 | 0.034351 | 27.36 |
| 6 Hours | 0.06819 | 0.06453 | 0.074069 | 0.06893 | 76.76 |
| Control | 0.034571 | 0.032915 | 0.026507 | 0.031331 | 23.04 |

TABLE 5B
Results of bioavailability test in Supernatant (Small Intestine)
of guinea pig

| | | | | | Concentration |
|---|---|---|---|---|---|
| | | | | | (ug/ml) (Y = |
| | | | | | 0.0007X + |
| Time | OD1 | OD2 | OD3 | Average | 0.0152) |
| 0 | 0.036135 | 0.04063 | 0.038485 | 0.038417 | 33.17 |
| 30 mins | 0.035252 | 0.021182 | 0.022579 | 0.026338 | 15.91 |
| Control | 0.020616 | 0.021508 | 0.017995 | 0.02004 | 6.91 |
| 1 Hour | 0.021445 | 0.02472 | 0.022229 | 0.022798 | 10.85 |
| Control | 0.024589 | 0.021682 | 0.025826 | 0.024032 | 12.62 |
| 4 Hours | 0.022667 | 0.024702 | 0.019184 | 0.022184 | 9.98 |
| Control | 0.023728 | 0.031897 | 0.019516 | 0.025047 | 14.07 |
| 6 Hours | 0.017626 | 0.007617 | 0.007499 | 0.010914 | −6.12 |
| Control | 0.031773 | 0.036568 | 0.026049 | 0.031463 | 23.23 |

Data for guinea pig stomach supernatant is shown in Table 5C and FIG. 7C. Results showed that concentration of RetroMAD1 is highest at 30 minutes after feeding; 20.33 μg/ml.

Concentration of RetroMAD1 starts to fall after 30 minutes from 18.55 μg/ml at 1 hour to 14.86 μg/ml at 4 hours and 7.77 μg/ml at 6 hours.

TABLE 5C
Results of bioavailability test in Supernatant (Stomach)
of guinea pig

| | | | | | Concentration |
|---|---|---|---|---|---|
| | | | | | (ug/ml) (Y = |
| | | | | | 0.0007X + |
| Time | OD1 | OD2 | OD3 | Average | 0.0152) |
| 0 | 0.029778 | 0.026176 | 0.026629 | 0.027528 | 17.61 |
| 30 mins | 0.027148 | 0.029376 | 0.031765 | 0.02943 | 20.33 |
| Control | 0.019232 | 0.031765 | 0.023121 | 0.024706 | 13.58 |
| 1 Hour | 0.026634 | 0.020743 | 0.037239 | 0.028205 | 18.58 |
| Control | 0.029548 | 0.020057 | 0.020743 | 0.023449 | 11.78 |
| 4 Hours | 0.032441 | 0.023119 | 0.021245 | 0.025602 | 14.86 |
| Control | 0.026809 | 0.020738 | 0.018296 | 0.021948 | 9.64 |
| 6 Hours | 0.021402 | 0.023904 | 0.016614 | 0.02064 | 7.77 |
| Control | 0.023544 | 0.021402 | 0.024692 | 0.023213 | 11.45 |

### Example 6

### Thermostability Trials

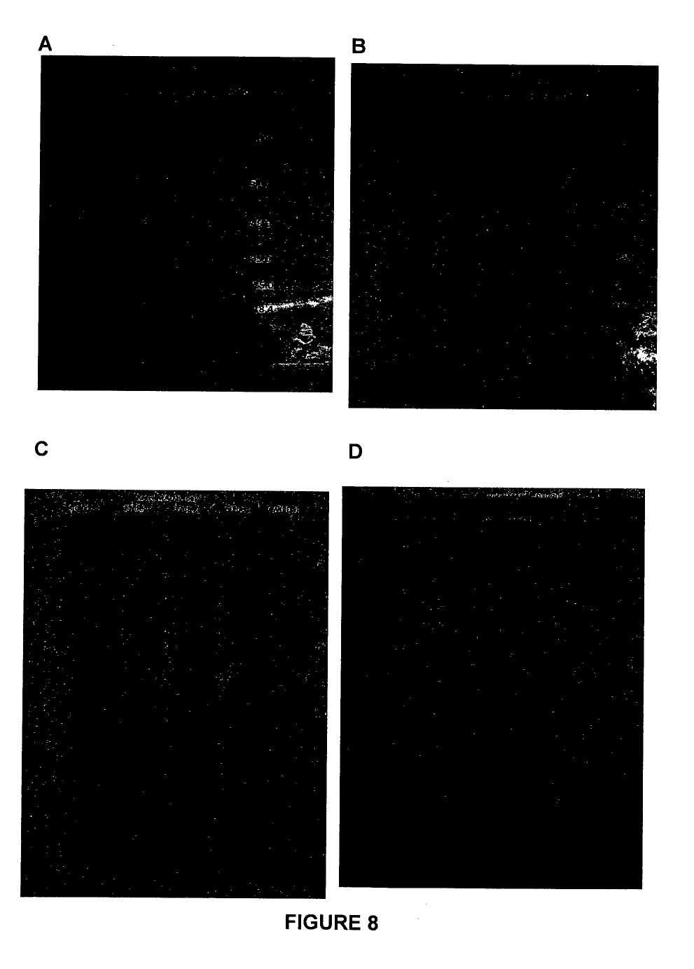

Protein stability under different temperatures was determined by keeping RetroMAD1 in multiple 1.5 ml Eppendorf tubes at 4° C. in a conventional refrigerator, 27° C.+/−1° C. in a laboratory which had 24 hour air-conditioning that maintained a narrow temperature range, in a conventional incubator oven set at 37° C. and in a laboratory oven set at 50° C. As RetroMAD1 is a protein of 41.2 kDa, running it on an SDS-PAGE gel and comparing the gel band of the sample stored at 4° C. with those kept at the other temperatures will reveal its stability. Up to day 7, the intensity of the gels remained the same irrespective of temperature up to 50C. Up to day 30, the intensity was similar for the samples stored at 4° C., 27+/−1° C. and 37° C. Unfortunately, a sample for 50° C. was not kept for the 30^{th} day. Based on the results as shown in FIG. 8, RetroMAD1 is stable up to 50° C. for a week and 37° C. for a month.

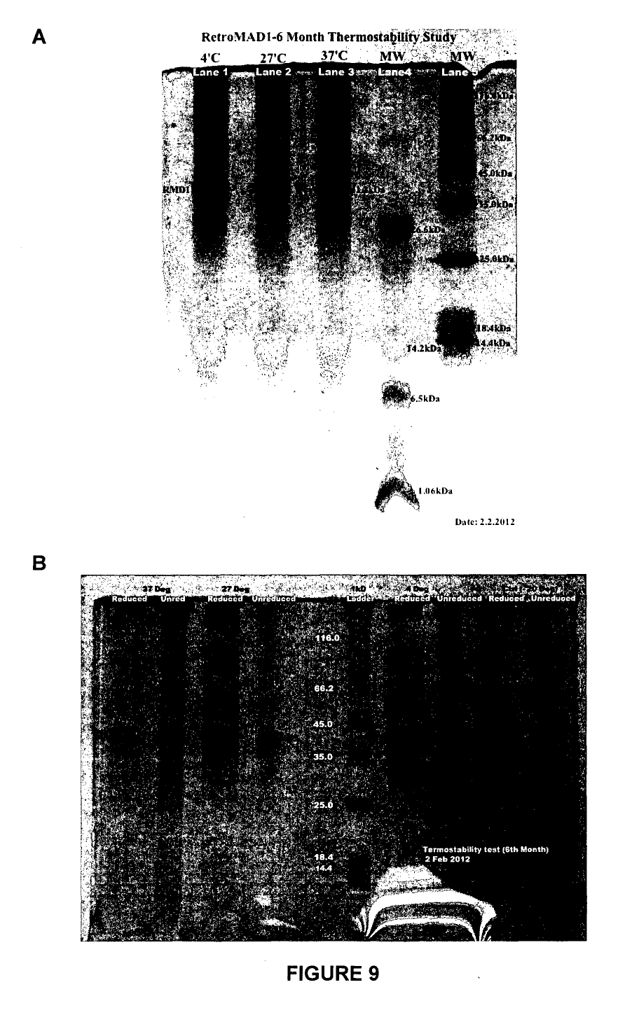

As shown in FIG. 9A, by using RetroMAD 1(RMD1) at 4° C. as a control, RMD 1 in 27° C. has overall similar amount and thickness of visible bands. There are no obvious or visible bands above 45.0 kDa for RMD1 in 37° C. compared to the control as well as RMD1 in 27° C.

Introducing a sample from −20° C. as a control actually to counter check the thermostability of sample from 4° C. which had been using throughout the experiment for 6 months duration showed clearly that the bands patterns on 27° C., 4° C. and −20° C. are similar while several cell debris bands were missing in 37° C. sample as shown in FIG. 9B. This confirms that RetroMAD1 can be stable up to 6 months.

### Example 7

### Stability Against Proteolytic Digestion

The ability of RetroMAD1 to withstand action of digestive enzymes acting at their pH optima is shown in Table 6 below.

50 mM DTT was prepared and added into pre-dissolved RetroMAD1 protein (1:1) made according to Example 1 and mixed. This was heated at 95° C. for 10 minutes and used to carry out enzyme assays with proteases such as Trypsin (pH8) (Lonza, Walkersville), α-Chymotrypsin (pH8) (Sigma-Aldrich) and Pepsin (pH2) (Sigma-Aldrich). After 10 minutes of heating at 95° C., the reaction was allowed to cool to room temperature (Approx. 10 mins) and proteases added to a final ratio of 1:100 (w/w) (protease:protein). This was incubated at 37° C. for 2 hours and protease activity terminated by incubating the mixture at 65° C. for 15 minutes. SDS-PAGE was used to analyze the fragments.

Other fusion proteins provided in Table 7 were made according to the method of Example 1 and the results of their fragmentation provided in Table 6A.

In particular, the stability of drugs, RetroGAD1 and Tamapal1, under gastric pH conditions and digestion of drugs by proteolytic enzymes such as trypsin, chymotrypsin and pepsin was determined.

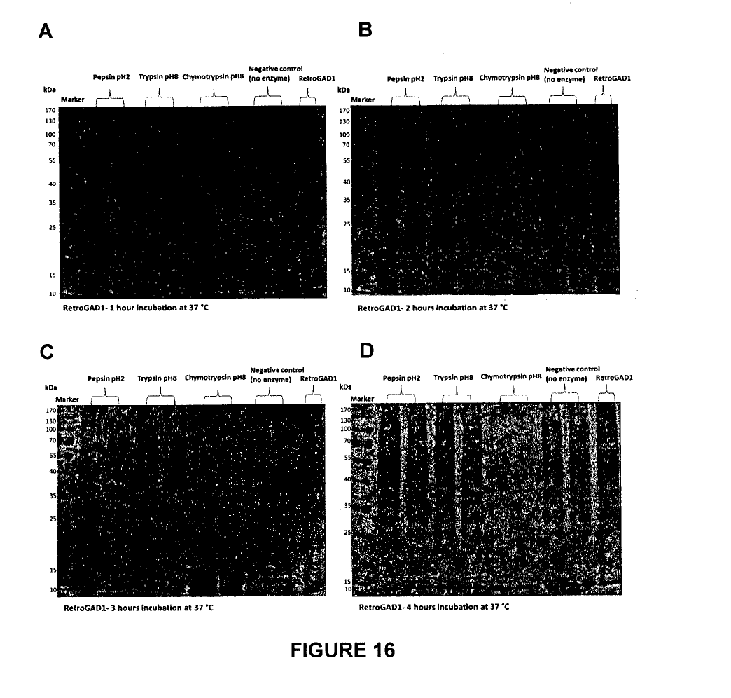

The stability of the drugs was tested by treating with proteases at various time points (1 hour, 2 hours, 3 hours and 4 hours) at 37° C. The integrity of the protein drugs were observed using SDS-page and compared to the control where the drugs are not treated with any protease. The results are provided in Table 6B below and FIGS. 16-18.

TABLE 6A
Results of fragmentation of fusion proteins according to the present invention

| | Size of | SEQ ID | | No of bands after protease digestion | | |
|---|---|---|---|---|---|---|
| Drug | drug | NO: | Structure of drug | Pepsin | Trypsin | Chymotrypsin |
| Amatilin | 40 kDa | 28 | A-B-C | No | No | No |
| (AM) | | | (AVBD103-MAP30-MYTILINC10C) | fragment | fragment | fragment |
| CT | 36 kDa | 29 | A-A-B-C | No | No | No |
| | | | (CERCROPIN A-CERCROPIN D- | fragment | fragment | fragment |
| | | | TAP29-DAP30-LATARCIN 2A) | | | |
| AB | 32 kDa | 30 | (RETROCYCLIN 101-MORMODICA | No | No | No |
| | | | ANTI-HIV PROTEIN 30) | fragment | fragment | fragment |
| BA | 32 kDa | 31 | (MORMODICA ANTI-HIV PROTEIN | No | No | No |
| | | | 30- RETROCYCLIN 101) | fragment | fragment | fragment |
| BC | 35 kDa | 32 | (MORMODICA ANTI-HIV PROTEIN | No | No | No |
| | | | 30- DERMASEPTIN 1) | fragment | fragment | fragment |
| CB | 35 kDa | 33 | DERMASEPTIN 1- MORMODICA | No | No | No |
| | | | ANTI-HIV PROTEIN 30 | fragment | fragment | fragment |
| Tamapal1 | 35.93 kDa | 34 | C-B-C | No | No | No |
| | | | TACHYPLESIN- MAP30- | fragment | fragment | fragment |
| | | | ALLOFERON1 | | | |
| K5 | 36.55 kDa | 35 | C-B-D | No | No | No |
| | | | (GAEGURIN 5-MAP30-(KLAKLAK)2 | fragment | fragment | fragment |
| RetroMAD1 | 41.2 kDa | 1 | A-B-C | No | No | No |
| | | | (RETROCYCLIN 101- MAP30- | fragment | fragment | fragment |
| | | | DERMASEPTIN 1) | | | |
| RetroGAD1 | 35.29 | 36 | A-B-C | No | No | No |
| | | | (RETROCYCLIN 101- GAP31- | fragment | fragment | fragment |
| | | | DERMASEPTIN 1) | | | |

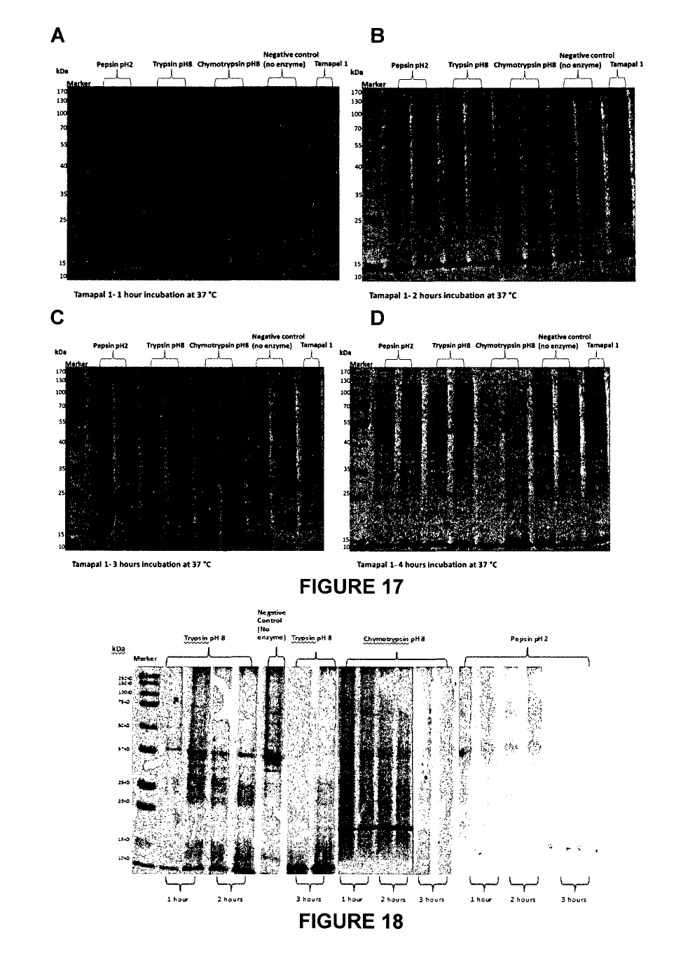

TABLE 6B
Summary of proteolytic digestion of RetroGAD1 (FIG. 16) and
Tamapal1 (FIG. 17) for 1 hour, 2 hours, 3 hours and 4 hours at
37° C. And RetroMAD1 (Figure 18) for 1 hour, 2 hours
and 3 hours at 37° C.

| | | Proteolytic enzyme | | |
|---|---|---|---|---|
| | | Pepsin | Trypsin | Chymotrypsin |
| Drug | Time | (pH 2) | (pH 8) | (pH 8) |
| RetroGAD1 | 1 hour | Not Digested | Not Digested | Digested |
| | 2 hours | Not Digested | Not Digested | Digested |
| | 3 hours | Not Digested | Not Digested | Digested |
| | 4 hours | Not Digested | Not Digested | Digested |
| Tamapal1 | 1 hour | Not Digested | Not Digested | Partially Digested |
| | 2 hours | Not Digested | Not Digested | Partially Digested |
| | 3 hours | Not Digested | Not Digested | Partially Digested |
| | 4 hours | Not Digested | Not Digested | Digested |
| RetroMAD1 | 1 hour | Not Digested | Not Digested | Not Digested |
| | 2 hours | Not Digested | Not Digested | Not Digested |
| | 3 hours | Not Digested | Partially | Partially Digested |
| | | | Digested | |

TABLE 7
Examples of fusion proteins according to the present invention

| SEQ | |
|---|---|
| ID | |
| NO: | SEQUENCE |
| 27 | [G-G-G-S]ₙ |
| 28 | SFGLCRLRRGFCAHGRCRFPSIPIGRCSRFVQCCRRVWVPGVGVPGVGGATGSDVNFDLSTATAKTYTK |
| | FIEDFRATLPFSHKVYDIPLLYSTISDSRRFILLNLTSYAYETISVAIDVTNVYVVAYRTRDVSYFFKE |
| | SPPEAYNILFKGTRKITLPYTGNYENLQTAAHKIRENIDLGLPALSSAITTLFYYNAQSAPSALLVLIQ |
| | TTAEAARFKYIERHVAKYVATNFKPNLAIISLENQWSALSKQIFLAQNQGGKFRNPVDLIKPTGERFQV |
| | TNVDSDVVKGNIKLLLNSRASTADENFITTMTLLGESVVNSCASRCKGHCRARRCGYYVSVLYRGRCYC |
| | KCLRCVPGVGVPGVG |
| 29 | LEKRKWKLFKKIEKVGQRVRDAVISAGPAVATVAQATALAKNVPGVGVPGVGGATGSDVSFRLSGATSK |
| | KKVYFISNLRKALPNEKKLYDIPLVRSSSGSKATAYTLNLANPSASQYSSFLDQIRNNVRDTSLIYGGT |
| | DVAVIGAPSTTDKFLRLNFQGPRGTVSLGLRRENLYVVAYLAMDNANVNRAYYFKNQITSAELTALFPE |
| | VVVANQKQLEYGEDYQAIEKNAKITTGDQSRKELGLGINLLITMIDGVNKKVRVVKDEARFLLIAIQMT |
| | AEAARFRYIQNLVTKNFPNKFDSENKVIQFQVSWSKISTAIFGDCKNGVFNKDYDFGFGKVRQAKDLQM |
| | GLLKYLGRPKSSSIEANSTDDTADVLVPGVGVPGVGKTCENLADTFRGPCFATSNC |
| 30 | MGRICRCICGRGICRCICGVPGVGVPGVGGSDVNFDLSTATAKTYTKFIEDFRATLPFSHKVYDIPLLY |
| | STISDSRRFILLDLTSYAYETISVAIDVTNVYVVAYRTRDVSYFFKESPPEAYNILFKGTRKITLPYTG |
| | NYENLQTAAHKIRENIDLGLPALSSAITTLFYYNAQSAPSALLVLIQTTAEAARFKYIERHVAKYVATN |
| | FKPNLAIISLENQWSALSKQIFLAQNQGGKFRNPVDLIKPTGERFQVTNVDSDVVKGNIKLLLNSRAST |
| | ADENFITTMTLLGESVVEFPW |
| 31 | MGSDVNFDLSTATAKTYTKFIEDFRATLPFSHKVYDIPLLYSTISDSRRFILLDLTSYAYETISVAIDV |
| | TNVYVVAYRTRDVSYFFKESPPEAYNILFKGTRKITLPYTGNYENLQTAAHKIRENIDLGLPALSSAIT |
| | TLFYYNAQSAPSALLVLIQTTAEAARFKYIERHVAKYVATNFKPNLAIISLENQWSALSKQIFLAQNQG |
| | GKFRNPVDLIKPTGERFQVTNVDSDVVKGNIKLLLNSRASTADENFITTMTLLGESVVEFPWVPGVGVP |
| | GVGGRICRCICGRGICRCICG |
| 32 | MGSDVNFDLSTATAKTYTKFIEDFRATLPFSHKVYDIPLLYSTISDSRRFILLDLTSYAYETISVAIDV |
| | TNVYVVAYRTRDVSYFFKESPPEAYNILFKGTRKITLPYTGNYENLQTAAHKIRENIDLGLPALSSAIT |
| | TLFYYNAQSAPSALLVLIQTTAEAARFKYIERHVAKYVATNFKPNLAIISLENQWSALSKQIFLAQNQG |
| | GKFRNPVDLIKPTGERFQVTNVDSDVVKGNIKLLLNSRASTADENFITTMTLLGESVVEFPWVPGVGVP |
| | GVGALWKTMLKELGTMALHAGKAALGAAADTISQGTQ* |
| 33 | MALWKTMLKELGTMALHAGKAALGAAADTISQGTQVPGVGVPGVGGSDVNFDLSTATAKTYTKFIEDFR |
| | ATLPFSHKVYDIPLLYSTISDSRRFILLDLTSYAYETISVAIDVTNVYVVAYRTRDVSYFFKESPPEAY |
| | NILFKGTRKITLPYTGNYENLQTAAHKIRENIDLGLPALSSAITTLFYYNAQSAPSALLVLIQTTAEAA |
| | RFKYIERHVAKYVATNFKPNLAIISLENQWSALSKQIFLAQNQGGKFRNPVDLIKPTGERFQVTNVDSD |
| | VVKGNIKLLLNSRASTADENFITTMTLLGESVVEFPW* |
| 34 | VPGVGVPGVGKWCFRVCYRGICYRRCRVPGVGVPGVGGATGSDVNFDLSTATAKTYTKFIEDFRATLPF |
| | SHKVYDIPLLYSTISDSRRFILLNLTSYAYETISVAIDVTNVYVVAYRTRDVSYFFKESPPEAYNILFK |
| | GTRKITLPYTGNYENLQTAAHKIRENIDLGLPALSSAITTLFYYNAQSAPSALLVLIQTTAEAARFKYI |
| | ERHVAKYVATNFKPNLAIISLENQWSALSKQIFLAQNQGGKFRNPVDLIKPTGERFQVTNVDSDVVKGN |
| | IKLLLNSRASTADENFITTMTLLGESVVNVPGVGVPGVGHGVSGHGQHGVHG |
| 35 | VPGVGVPGVGFLPLLAGLAANFLPTIICFISYKCVPGVGVPGVGGATGSDVNFDLSTATAKTYTKFIED |
| | FRATLPFSHKVYDIPLLYSTISDSRRFILLNLTSYAYETISVAIDVTNVYVVAYRTRDVSYFFKESPPE |
| | AYNILFKGTRKITLPYTGNYENLQTAAHKIRENIDLGLPALSSAITTLFYYNAQSAPSALLVLIQTTAE |
| | AARFKYIERHVAKYVATNFKPNLAIISLENQWSALSKQIFLAQNQGGKFRNPVDLIKPTGERFQVTNVD |
| | SDVVKGNIKLLLNSRASTADENFITTMTLLGESVVNVPGVGVPGVGKLAKLAK KLAKLAK |

The human G.I. is divided into the oral cavity, the stomach, the small intestines and the large intestines. Protease enzymes occur in the stomach, in the form of pepsin, and in the front part of the small intestines called the duodenum, in the form of trypsin and chymotrypsin. Pepsin is most active at pH 2 while trypsin and chymotrypsin are most active at pH 8. By running SDS-PAGE gels after incubation with the respective enzyme at its pH optima, single bands corresponding to the correct molecular size indicated that no enzymatic breakdown was observed for that period of incubation. Based on the results in the table 6 below, several compounds of this class demonstrated this attribute for a 2 hour incubation period with pepsin, trypsin and chymotrypsin individually because food does not normally retain in either the stomach or the duodenum for longer than 2 hours. This 2 hour incubation period for a drug to be orally administered before meals is far more than sufficient to prove stability within the G.I. with regard to enzymatic cleavage.

Conjugating these peptides with MAP30, surprisingly rendered the fusion protein stable for oral administration as shown in its ability to survive protease digestion.

Also, RetroGAD1 and Tamapal1 were not digested by pepsin (pH2) and trypsin (pH8) after 1 hour, 2 hours, 3 hours and 4 hours of digestion. Conversely, RetroGAD1 and Tamapal1 were digested by chymotrypsin (pH8) at different points of time. RetroGAD1 was digested by chymotrypsin after 1 hour, Tamapal1 was only partially digested after 3 hours and digested after 4 hours. For RetroMAD1, it was not digested by pepsin (pH2), chymotrypsin (pH8) and trypsin (pH8) up to 2 hours. These results indicated that Tamapal1 and RetroMAD1 are the most stable drugs, followed by RetroGAD1. Hence, the stability of the three drugs under in vitro gastric conditions based on the results is RetroMAD1>Tamapal1>RetroGAD1. The significant outcome of this study is to develop an understanding on the stability of the drugs (RetroMAD1, RetroGAD1 and Tamapal1) in human digestive system, thus allows oral drug delivery.

### Example 8

### Expression Profile of HSV-Infected Cells Treated with RetroMAD1

4 sets of cells were prepared:

1. Vero Cells

2. Vero Cells+RetroMAD1

3. Vero Cells+Virus

4. Vero Cells+RetroMAD1+Virus

*Time point of the sample preparation is 72 hours

Vero cells (African Green monkey kidney cell line) were obtained from American Type Culture Collections, Rockville, Md. They were used as the host cells for HSV-2. The cells were cultured using Dulbeco's Modified Eagle Medium (DMEM), supplemented with 10% Foetal bovine serum (FBS).

Herpes simplex 2 (HSV-2) virus stocks were obtained by inoculating monolayer of Vero cells in a 75 cm² tissue culture flasks with virus in maintenance medium containing 2% FBS and the cells were allowed to continue propagating at 37° C. for 4 days until the cytopathic effect (CPE) are confirmed. The cells and supernatant were then harvested by gentle pipetting. The media was removed from the flasks. 4 mL of trypsin added to each flask and placed back in incubator for 5 minutes. The flasks were removed from incubator and 4 mL of media added to each flask to inactivate trypsin. Cells were collected into 15 mL tubes and spun at 3000 rpm for 5-10 minutes at room temperature. The supernatant was removed from 15 ml tubes and 5 mL of PBS added to each tube. The cells were resuspended in PBS to remove excess trypsin and media. The cells were spun at 3000 rpm for 5-10 minutes at room temperature. The supernatant was removed from tubes and 1 mL of fresh lysis buffer added to each tube. The cells were resuspended in fresh lysis buffer and place the tubes in at 4° C. for 2-4 hours. The cell lysates were transferred to 1.5 mL microcentrifuge tubes and spun at 40000 rpm for 1 hour at 4° C. The supernatant was finally removed and transferred to a clean microcentrifuge tube and the remaining lysate stored in −80° C. freezer. The protein concentration was determined according to the instructions of GE Healthcare 2D quant kit. A standard curve (0-50 μg) was prepared using 2 mg/ml BSA standard solution and the protein concentration determined using the standard curve. Drystrips were rehydrated according to a method known in the art and first dimension isoelectric focusing carried our using the IPGphor Regular Strip Holder. Equilibration was carried our and then second dimension gel electrophoresis carried out by preparing 12.5% stacking gel and placing the strips on top of the stacking gel. Filter paper was loaded with protein marker on the stacking gel by making a well and the gel run at 120V. Mass spectrometry analysis was then carried out by first staining the gels and then destaining them. The gels were analysed using PDQQuest Software. The gels obtained for the 4 sets of cells above were compared and the protein spots with at least 2 fold increase or decrease in intensity were picked. These protein spots were analysed using MALDI TOF-TOF and search against MASCOT database done to retrieve protein spot identity. MASCOT search results that gave protein scores greater than 51 were considered significant. UniProt was then used to identify the function of the protein.

The results, in particular, the ability of RetroMAD1 to up-regulate cellular pathways in normal and virally infected cells is shown in Table 8 below. Influence of gene expression at a cellular level is proof of RetroMAD1's ability to penetrate and be readily absorbed by cells.

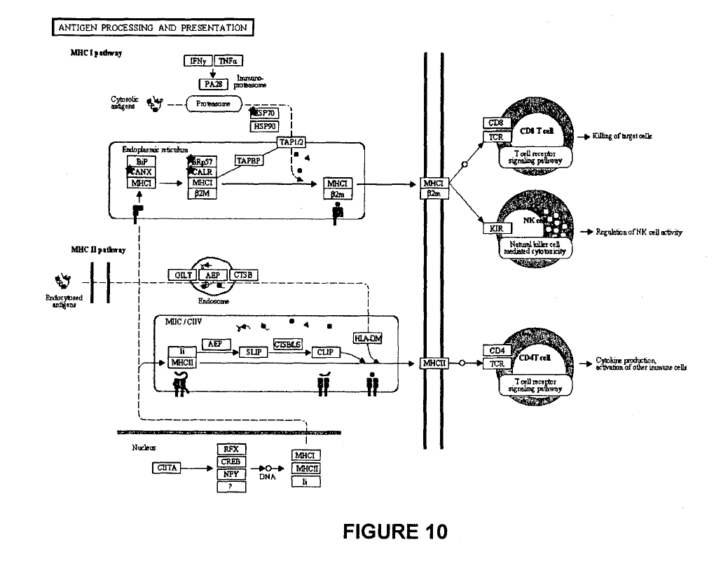

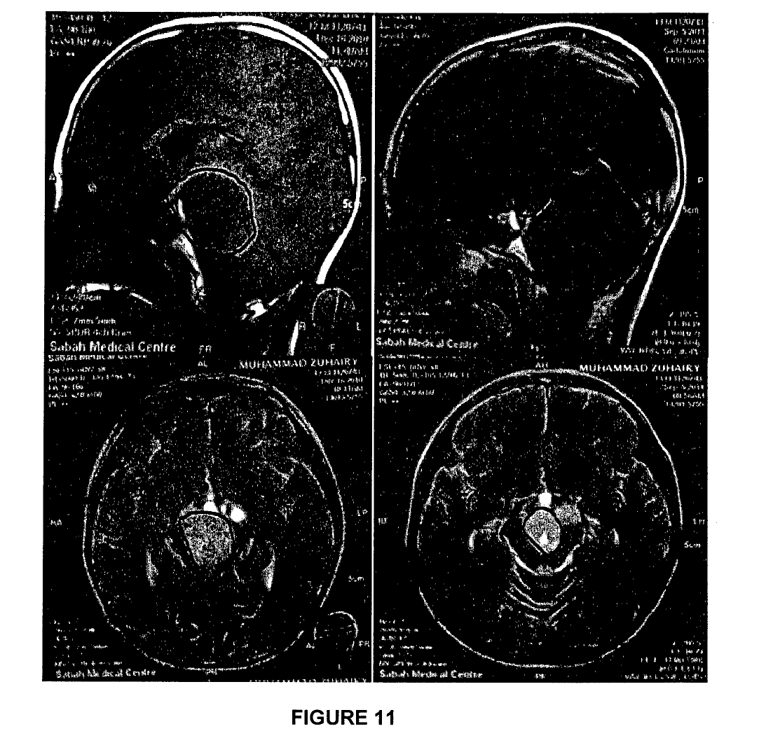

Viruses are known to hijack the cell's machinery to its advantages and major histocompatibility (MHC) class 1 antigen presentation molecules are usually targeted due to its important role in the immune system. From the Table 8 it was evident that the virus had down-regulated the expression of proteins (sequestosome-1, calnexin, heat shock cognate, calreticulin, endoplasmin and protein disulfide-isomerase) involved in the MHC class I pathway. This was confirmed in FIG. 10 where the proteins were uploaded on david.abcc.ncifcrf.gov to produce the related pathways.

However, the expression of these proteins was augmented after the cells were treated with RetroMAD1. Sequestosome-1, a protein responsible in the aggregation of a key initiator caspase, CASP8; was observed to be significantly up-regulated by as much as 11-fold. Alpha-enolase, a protein with glycolytic function as well as patholphysiological roles in many eukaryotes processes was also significantly suppressed by the virus. However, the expression of this protein was induced upon treatment with RetroMAD1. In addition to alpha-enolase, annexin Al was observed to be similarly repressed by the virus and its expression was restored upon treatment with the compound. Annexin Al is a calcium-dependent phospholipid-binding protein which plays an important role in cellular processes such as proliferation and apoptosis as well as in preventing the fusion of raft-associated vesicles at selected membrane domains.

Among the differentially expressed proteins, nucleoside diphosphate kinase with an ability in regulating cell cycle was also restored in treated cells and this is suggestive that RetroMAD1 would be able to re-establish chromosomal stability in virally infected cells. RetroMAD1 is presumed to target the MHC class I pathway's proteins where it helps to re-establish the cell's ability in presenting viral peptides to the T-cells and ensure viral elimination in the immune system.

TABLE 8
Expression profile of HSV-infected cells treated with RetroMAD1

| Entrez | | | | RetroMad1 treated | Virally | RetroMad1 treated |
|---|---|---|---|---|---|---|
| ID | Protein Accession | Protein | Pathway involved | healthy cells | infected cells | virally infected cells |
| 8878 | SQSTM_PONAB | Sequestosome-1 | — | +1.01 | −8.47 | +11.06 |
| 821 | CALX_PONAB | Calnexin | Antigen processing and | −2.51 | −3.77 | +6.17 |
| | | | presentation, interaction in | | | |
| | | | MAPK3/ERK1 | | | |
| 811 | CALR_CHLAE | Calreticulin | Cell cycle | +2.56 | −1.07 | +3.65 |
| 3312 | HSP7C_SAGOE | Heat shock cognate | Antigen processing and | −1.80 | −9.07 | +2.00 |
| | | protein | presentation | | | |
| — | PDIA1_MACFU | Protein disulfide- | — | +1.87 | −5.03 | +2.03 |
| | | isomerase | | | | |
| — | ENPL_MACFA | Endoplasmin | IL6-mediated signaling | +2.02 | −3.64 | +4.34 |
| 2023 | ENOA_PONAB | Alpha-enolase | — | −1.56 | −6.32 | +1.30 |
| 301 | ANXA1_PANTR | Annexin A1 | — | −2.31 | −7.70 | +2.29 |
| — | NDKB_PONAB | Nucleoside diphosphate | — | +1.55 | −1.11 | +2.48 |
| | | kinase | | | | |
| 4691 | NUCL_PONAB | Nucleolin | — | −1.55 | −10.04 | +17.89 |

### Example 9

### Preliminary Screening Against Lung Cancer Cell Lines (A549) and Breast (MCF-7) Cancer Cell Lines

### Normal and Cancer Cell Lines

Cell lines used in this study were established cell lines. The human breast carcinoma (MCF-7), human lung carcinoma (A549), human normal breast epithelium (184B5) and human normal bronchus epithelium (NL20) were purchased from the American Type Tissue Culture Collection, Manassas, USA. A549 and MCF-7 were grown in RPMI-1640 (Roswell Park Memorial Institute) and DMEM (Dulbecco's modified Eagles Medium), respectively while NL20 and 184B5 were grown in F-12K (ATCC, USA) and Mammary Epithelial Growth Medium (Lonza), respectively. Growth media was supplemented with 10% heat-inactivated foetal bovine serum (FBS, Gibco). Cells were maintained in humidified air with 5% CO₂ at 37° C. Cells undergoing exponential growth were used throughout the experiments.

### Determination of Cell Viability, Growth Inhibition and Half-Maximal Inhibitory Concentration (IC₅₀)

The anti-proliferative activities of RetroMAD1 were measured using a colorimetric MTS assay which is composed of solutions of a novel tetrazolium compound 3-(4,5-dimethylthiazol-2-yl)-5-(3-carboxymethoxyphenyl)-2-(4-sulphonyl)-2H-tetrazolium, inner salt, MTS and an electron coupling reagent (phenazine methosulphate; PMS) (Promega, Madison, Wis.). This assay is based on the cleavage of the yellow dye MTS to purple formazan crystals by dehydrogenase activity in mitochondria, a conversion that occurs only in living cells. Prior to each experiment, cells from a number of flasks were washed thoroughly with phosphate buffered saline (PBS) (1×), harvested by treatment at 37° C. with a solution of Trypsin-EDTA (1×) and re-suspended in the culture medium. The cells were then counted and were seeded in each well of a 96-well flat-bottom plate at a concentration of 1×10⁴ cells/well for MCF-7, A549 and 184B5 cells and 2×10⁴ cells/well for NL20 cells. After 24 h of incubation at 37° C. with 5% CO₂, the cells were treated with various concentrations of RetroMAD1 for 24, 48 and 72 h. Control wells received culture medium without RetroMAD1 and blank wells contained culture medium with different concentrations of RetroMAD1 without cells. After 24, 48 and 72 h of incubation, cell proliferation was determined by the colorimetric MTS assay. Briefly, 20 μl per well of MTS reagent was added to the plates and incubated at 37° C. for 1 h in a humidified 5% CO₂ atmosphere. The intensity of formazan, reduced product of MTS after reaction with active mitochondria of live cells, was determined by measuring the absorbance at a wavelength of 490 nm using GloMax Multi Detection System (Promega, USA). Absorbance is directly proportional to the number of live cells in the culture. At least three replications for each sample were used to determine the anti-proliferative activity. Percentages of cell viability and growth inhibition were calculated using the following formulas:

$$Percentage of cell viability = \frac{[ Mean O D of the test group - Mean O D of the blank group ]}{[ \begin{matrix}Mean O D of the control group - \\ Mean O D of the blank group\end{matrix} ]} \times 100 \%$$

Percentage of growth inhibition=100%−Percentage of cell viability

The IC₅₀ value (the concentration of drug that inhibits cell growth by 50% compared to untreated control) was determined from the dose response curve of the anti-proliferative activity with cell viability (Y-axis) against concentrations of RetroMAD1 (X-axis). Comparative study of the 24-hr IC50 values between a normal and a cancerous lung cell line gave an experimental Therapeutic Index of 2.94. The results are shown in Table 9 below.

TABLE 9
IC50 results of RetroMAD1 on breast can lung cancer cell lines.

| IC₅₀ (μg/mL) of RetroMAD1 | |
|---|---|
| Breast Cells | Lung Cells |

| | Cancer - | Normal - | Cancer - | Normal - |
|---|---|---|---|---|
| | Human | human | Human | Human |
| | breast | breast | lung | bronchus |
| | carcinoma | epithelium | carcinoma | epithelium |
| Time | (MCF-7) | (184B5) | (A549) | (NL20) |
| 24 h | 94.0 | ≦500.0 | 109.0 | 321.0 |
| 48 h | 78.5 | 180.0 | 80.0 | 254.0 |
| 72 h | 77.0 | 90.0 | 80.0 | 164.5 |

### Example 10

### RetroMAD1 was Tested on a Patient with a Pontine Glioblastoma

A 13-year old ethnic Malay boy presenting a case of pontine glioblastoma was treated for 5 months using oral RetroMAD1 at 0.2 mg/kg body weight with informed consent on compassionate grounds. He was first diagnosed in December 2010 after severe bouts of vomiting several times a day with a maximum of 14×/day. The initial MRI revealed a 5 cm diameter pontine globlastoma that exerted pressure upon the brain necessitating installing a EVD (Extra Ventricular Drainage) shunt to drain excess CSF (Cerebrospinal Fluid) from the ventricular space into the stomach. The tumour was considered to be inoperable without extreme risk and radiation was opted for without chemotherapy. Radiation therapy was carried out in February 2011 and when no significant improvements were noted, the father of the child was told that the child might have only a few months to live. The father of the boy applied for use of RetroMAD1 as an experimental drug and treatment began at 0.2 mg/kg body weight per dose taken before food with water to dilute the RetroMAD1 on an empty stomach three times a day on a daily basis. After a week, the boy mentioned that all headaches had ceased and began to return to schooling and even stopped the use of steroids. He remained fairly asymptomatic for the next 5 months while he was on the above mentioned dosage regime of RetroMAD1 and when another MRI was taken, it was noted that the pontine glioblastoma had shrunk from a 5 cm diameter tumour to an approximately 2.5 cm diameter tumour. Comparing his blood profile before and after RetroMAD1 treatment (Table 10), it appeared that only alkaline phosphotase was above the normal range at 166 IU/L, however, before treatment, it was even higher at 204 IU/L. In order to protect the confidentiality of the patient, the details of the patient have been undisclosed.

TABLE 10
Blood results of patient

| | Result | | | |
|---|---|---|---|---|
| | Before | After | | |
| Test Name | Treatment | Treatment | Unit | Normal Range |

FULL BLOOD COUNT

| | | | | |
|---|---|---|---|---|
| Haemoglobin | 13.5 | 12.9 | gm % | 12.5-17.5 |
| RCC | 4.7 | 4.5 | ×10\hat{ }12/L | 4.5-6.0 × 10\hat{ }12 |
| PCV | 39 | 39 | % | 40.0-50.0 |
| MCV | 83 | 87 | fl | 78-97 |
| MCH | 29 | 29 | pg | 26-34 |
| MCHC | 34 | 33 | g/dl | 31-37 |
| RDW | 13 | 15 | % | <16 |

TOTAL WHITE
DIFF. COUNT

| | | | | |
|---|---|---|---|---|
| Total WCC | 13 | 6.2 | ×10\hat{ }9/L | 4.0-11.0 × 10\hat{ }9 |
| Neutrophils | 70.4 | 58.2 | % | 40-74 |
| Lymphocytes | 20.3 | 34.3 | % | 20-45 |
| Monocytes | 8.2 | 5.5 | % | 3.4-7 |
| Eosinophils | 1 | 1.9 | % | 0-7 |
| Basophils | 0.1 | 0.1 | % | <1.5 |
| Platelets | 370 | 323 | ×10\hat{ }9/L | 150-400 × 10\hat{ }9 |
| ESR | nil | 13 | mm/hr | 0-15 |

PERIPHERAL BLOOD
FILM

| | | |
|---|---|---|
| HB | nil | Normal |
| RBC | nil | Normochromic normocytic |
| WBC | nil | Morphologically normal |
| Platelets | nil | Adequate |
| IMPRESSION | nil | Normal blood film |

LIPID PROFILE

| | | | | |
|---|---|---|---|---|
| Total Cholestrol | nil | 3.6 | mmol/L | <5.2 |
| HDL Chol | nil | 1.2 | mmol/L | >0.9 |
| LDL Col | nil | 2.2 | mmol/L | <2.6 |
| Triglycerides | nil | 0.4 | mmol/L | <1.7 |
| Chol/HDL Ratio | nil | 3.0 | | <4.5 |
| Fasting Glucose | 5.5 | 5.0 | mmol/L | 3.5-5.4 |
| Uric acid | nil | 0.35 | mmol/L | 0.20-0.43 |

RENAL FUNCTION TEST

| | | | | |
|---|---|---|---|---|
| Urea | 3.6 | 2.2 | mmol/L | 1.7-8.5 |
| Creatinine | 57 | 81 | umol/L | 50-120 |
| Inorganic Phosp | nil | 1.4 | mmol/L | 0.8-1.6 |
| Calcium | nil | 2.4 | mmol/L | 2.0-2.6 |

ELECTROLYTE

| | | | | |
|---|---|---|---|---|
| Sodium | 131 | 141 | mmol/L | 135-148 |
| Potassium | 4.1 | 3.8 | mmol/L | 35-5.2 |
| Chloride | 94 | 106 | mmol/L | 94-111 |

LIVER FUNCTION TEST

| | | | | |
|---|---|---|---|---|
| Total protein | 80 | 70 | g/L | 66-87 |
| Albumin | 45 | 40 | g/L | 34-54 |
| Globulin | 35 | 30 | g/L | 18-42 |
| A/G ratio | 1.3 | 1.3 | | 1.0-2.2 |
| Total Bilirubin | 4 | 5 | umol/L | <20.0 |
| AST (SGOT) | 18 | 18 | IU/L | 15-37 |
| ALT (SGPT) | 30 | 26 | IU/L | 8-65 |
| ALP | 204 | 166 | IU/L | 50-136 |
| GGT | 25 | 20 | IU/L | 11-85 |

IMMONOLOGY/SEROLOGY

| VDRL | nil | Non-reactive | | |
|---|---|---|---|---|
| Hep Bs Ag | nil | Non-reactive | | |
| Hep Bs Ab | nil | Non-reactive | | |
| HIV I&II (EIA) | nil | Non-reactive | | |
| AFP | nil | 2.1 | IU/ml | <12 |
| TSH | 0.36 | 0.9 | mIU/L | 0.30-5.50 |

BLOOD GROUPING

| | | |
|---|---|---|
| Blood Group | nil | O |
| Rhesus Factor | nil | Positive |
| Rheumatoid F | nil | Negative |

URINE ANALYSIS

| | | | |
|---|---|---|---|
| Colour | nil | Yellow | |
| Specific Gravity | nil | 1.02 | 1.005-1.025 |
| pH | nil | 6 | |
| nitrites | nil | Negative | |

+

| | | |
|---|---|---|
| Protein | nil | |
| Glucose | nil | Negative |
| Ketone | nil | Negative |
| Blood | nil | Negative |
| Urobilinogen | nil | Normal |
| Bilirubin | nil | Negative |

MICROSCOPY

| | | | |
|---|---|---|---|
| WBC/hpf | nil | 0-1 | ×10\hat{ }6/L |
| RBC/hpf | nil | 0-1 | ×10\hat{ }6/L |
| Epithelial Cells | nil | Not Seen | |
| Casts | nil | Not Seen | |
| Crystals | nil | Not Seen | |

### Example 11

### Pharmacokinetic Study for Various Drugs of the Present Invention

Mice pK study is the study of the pharmacokinetics of the drug. pK includes study of the absorption, distribution, metabolism and excretion. Pharmacokinetics of RetroMAD1, RetroGAD1, and Tamapal1 (as provided in Table 1c) was studied in ICR strain mice aged between 4-6 weeks.

The pharmacokinetic data of RetroMAD1, RetroGAD1, and Tamapal1 was derived in 6-8 weeks female ICR mice. For each PK study for RetroMAD1, RetroGAD1, and Tamapal1, 81 mice were administered with single dose of 70 ml per mouse which is a 50× dose of 0.2 mg/kg body weight for RetroMAD1, 0.7 ml per mouse for RetroGAD1, and 1 ml per mouse for Tamapal1. These drugs were given orally at time points, 0.5-, 1-, 2-, 4-, 8- and 12-hours on Day 1 and daily for Day 2, 3, 4, 5, 6, 7 and 10. Prior to administering the drug, the mice will be starved for 2 hours. At these time points, 0.5-, 1-, 2-, 4-, 8- and 12-hours on Day 1 and at Day 2, 3, 4, 5, 6, 7 and 10, 3 mice were fed orally with the drug (as treatment) and 3 mice were fed with water (as control). Before bleeding, each mouse was given 0.15 mL of anesthetic drug (Ketamine and Xylazine) via intraperitoneal injection. Each day blood samples were drawn from the heart of three treated mice and three controls at each time point. For the first day after the feed, the blood was collected after 30 min, 1 hour, 2 hours, 4 hours, 8 hours and 12 hours after oral administration and for the following days (up to day 10) the blood was collected just 30 min after administration. The blood samples were centrifuged and the serum was collected for ELISA. This was to determine the concentration of the drug in the blood system upon feeding (drug vs. water). Also, the organs including stomach, small and large intestine, liver and kidney were harvested. Harvested organs were homogenized in PBS and centrifuged to collect the supernatants. These supernatants were filtered and used for ELISA. Direct ELISA was used to determine concentration of RetroGAD1, and Tamapal1 in the blood serum, stomach, liver, kidney and intestine, while a capture ELISA was used for RetroMAD1.

A direct ELISA was used for detecting RetroGAD1 and Tamapal1 in mice Sera. In direct ELISA, a 96-well U-bottomed was coated with 5 μl of samples of mouse serum, supernatant of stomach, liver, kidney and intestine with 95 μof coating buffer (0.2 M sodium carbonate-bicarbonate, pH 9.6). The sample coated plate was incubated at 4° C. overnight. Plates were washed six times with 0.05% Tween-20 in PBS 1×. 100 ul/well of rabbit anti-RetroGAD1/Tamapal1 antibody diluted 1:500 in 5% BSA in PBS and were added to the wells. After incubation at 37° C. for 1 hour, plates were washed similarly and 100 μl/well of anti-rabbit IgG diluted 1:10000 in 5% BSA in PBS was added. After incubation at 37° C. for 1 hour, plates were washed and 100 μl/well streptavidin-HRP diluted 1:10000 in 5% BSA in PBS was added. After incubation at 37° C. for 1 hour in the dark, plates were washed and 100 μl/well of OPD added to each well. Plates were incubated in the dark for 30 minutes at room temperature and reaction stopped with 50 μl/well of 4N H2SO4. Optical density (OD) for each sample was measured at 490 nm and 600 nm as background. A standard curve was then generated by doing the direct ELISA as described above with RetroGAD1 and Tamapal1 of ½ dilution, the concentrations of RetroGAD1, and Tamapal1 at 100, 50, 25, 12.5, 6.25, 3.125, 1.6, 0.8, 0.4, 0.2 and 0.1 μg/ml. The equation of the standard curve was used to determine concentration of RetroGAD1, and Tamapal1 in serum, stomach, liver, kidney and intestine.

ELISA for detecting RetroMAD1 in mice Sera is an in house Capture ELISA with anti-human-IgG-HRP. To prepare the capture antibody, a cat was fed daily with RetroMAD1 and after 6 months, blood was harvested and serum extracted. This serum was used as the capture antibody. 100 μl/well of this polyclonal cat anti-RetroMAD1 antibody diluted 1:80 in coating buffer (0.2 M sodium carbonate-bicarbonate, ph 9.6) was adsorbed onto 96-well polystyrene ELISA plates. The plates were incubated at 4° C. overnight. Plates were washed three times with 0.05% Tween-20 in PBS 1×. 100 μl/well of mice serum diluted 1:2 in 0.05% BSA in PBS and were added to the wells. After incubation at 37° C. for 1 hour, plates were washed similarly and 100u1 of anti RetroMAD1 positive human serum diluted 1:2000 in 0.05% BSA in PBS was added. After incubation at 37° C. for 1 hour, plates were washed and 100 μl/well Rabbit anti-human IgG HRP conjugate diluted 1:6000 in 0.05% BSA in PBS, was added. After incubation at 37° C. for 1 hour in the dark, plates were washed and 100 μl/well of OPD added to each well. Plates were incubated in the dark for 30 minutes at room temperature and reaction stopped with 50 μl/well of 4N H2SO4. Optical density (OD) for each sample was measured at 490 nm and 600 nm as background. All OD readings were then converted to Log values to obtain concentrations in μg/ml and the standard curves.

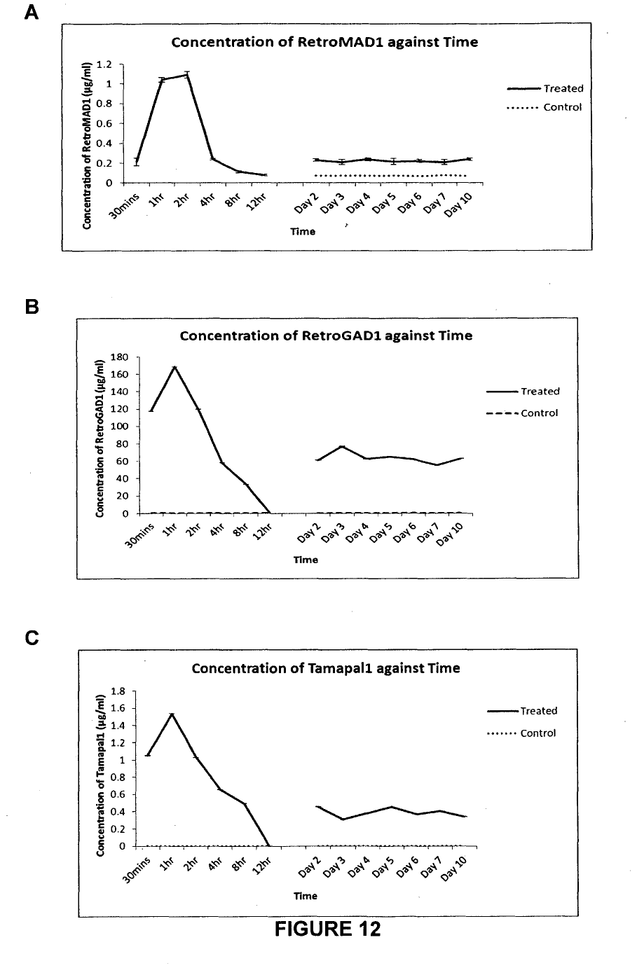

The mice pK results for RetroMAD1 are shown in FIG. 12A. The pK data showed that RetroMAD1 was detected in the serum as early as 30 minutes post feeding at about 0.2 μg/ml that reached a maximum at 1-2hours at 1-1.1 μg/ml before dropping again to about 0.2 μg/ml at 4 hours. By 12 hours post feeding, levels were almost similar to the unfed controls indicating that the protein had been completely metabolized. Subsequent daily sampling at 30 minutes post feeding indicated levels around 0.2 μg/ml.

The mice pK data for RetroGAD1 are shown in FIG. 12B. The results showed that RetroGAD1 was detected in the serum as early as 30 minutes post feeding at about 118 μg/ml that reached a maximum at 1 hour at 169 μg/ml and 120 μg/ml before dropping again to 58.3 μg/ml at 4 hours and 33.7 μg/ml at 8 hours. By 12 hours post feeding, levels were similar to the unfed controls indicating that the drug had been completely eliminated from the blood. Subsequently daily sampling at 30 minutes post feeding indicated levels around 50 μg/ml.

The mice pK data for Tamapal1 are shown in FIG. 12C The results showed that Tamapal1 was detected in the serum as early as 30 minutes post feeding at about 1.05 μg/ml that reached a maximum at 1 hour at 1.54 μg/ml and 1.03 μg/ml before dropping again to 0.656 μg/ml at 4 hours and 0.493 μg/ml at 8 hours. By 12 hours post feeding, levels were similar to the unfed controls indicating that the drug had been completely eliminated from the blood. Subsequently daily sampling at 30 minutes post feeding indicated levels around 0.45 μg/ml.

Subsequent daily sampling 30 minutes post feeding levels around 0.2 μg/ml for RetroMAD1, 50 μg/ml for RetroMAD1, and 0.45 μg/ml Tamapal1, these data suggest bioavailability of the drugs.

### Example 12

### Organ Pharmacokinetics for RetroMAD1, RetroGAD1 and Tamapal1 in Mice

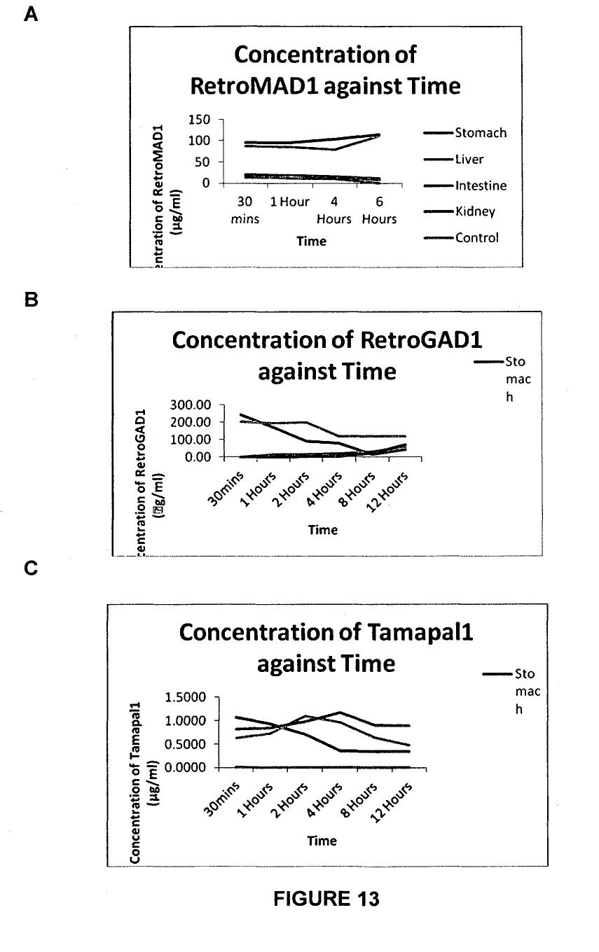

Mice Pk data of stomach, liver, kidney and intestine studies the pharmacokinetics of the drug. From the results as shown in FIGS. 13A-C after RetroGAD1 and Tamapal1 are each orally given to mice and RetroMAD1 orally given to guinea pigs; these drugs were absorbed into the stomach and then distributed into the blood. Subsequently, metabolized and excreted in the kidney and intestine.

For RetroMAD1, pK study was carried out in guinea pigs. Data for guinea pigs small intestine supernatant is shown in Table 11 and FIG. 13A. Results showed that thr highest concentration of RetroMAD1 was detected at 30 minutes at about 16 μg/ml. The concentration of RetroMAD1 then started to decrease to about 11 μg/ml at 1 hour, and to 9 μg/ml at 4 hours. The protein drug was then released from the small intestine at 6 hours where no RetroMAD1 was detected.

TABLE 11
Concentration of RetroMAD1 in guinea pig stomach, liver,
intestine and kidney after oral administration of
RetroMAD1 at 30 mins, 1 hours, 4 hours and 6 hours

| | Concentration of RetroMAD1 (μg/ml) | | | | |
|---|---|---|---|---|---|
| Time | Stomach | Liver | Kidney | Small Intestine | Control |
| 30 mins | 20.33 | 86.28 | 94.59 | 15.91 | 13.58 |
| 1 Hour | 18.58 | 85.86 | 94.91 | 10.85 | 12.62 |
| 4 Hours | 14.86 | 78 | 102.68 | 9.98 | 14.07 |
| 6 Hours | 7.77 | 111.22 | 114.12 | 0 | 11.45 |

As for RetroGAD1, result showed (Table 12 and FIG. 13B) that the drug was absorbed into the stomach and blood system. The concentration of RetroGAD1 was detected at 30 minutes at 241.50 μg/ml in the stomach. Then the concentration in the stomach started to drop to about 170.47 μg/ml at 1 hour, and 92.62 μg/ml at 2 hours. RetroGAD1 was then released into the blood system at 1-2 hours and the concentration peaked at 1-2 hours at 169 μg/ml and 120 μg/ml. RetroGAD1 begun to increase in the liver from 2-4 hours and was detected to be 118.66 μg/ml. in the intestine, RetroGAD1 started to peak from 8 and 12 hours at 31.90 μg/ml and 60.15 μg/ml respectively. RetroGAD1 was also detected in the kidney at 22.02 μg/ml and 68.93 μg/ml at 8 hours and 12 hours respectively.

TABLE 12
Concentration of RetroGAD1 in stomach, liver and intestine at after
oral administration of RetroGAD1 at 0.5, 1, 2, 4, 8, 12 hours

Concentration of RetroGAD1 (μg/ml)

| Time | Stomach | Liver | Intestine | Control |
|---|---|---|---|---|
| 30 mins | 241.50 | 202.61 | 0.00 | 0.00 |
| 1 Hours | 170.47 | 192.71 | 16.63 | 0.00 |
| 2 Hours | 92.62 | 198.53 | 16.73 | 3.50 |
| 4 Hours | 80.10 | 118.66 | 18.60 | 3.50 |
| 8 Hours | 15.82 | 117.89 | 31.90 | 22.02 |
| 12 Hours | 41.13 | 117.89 | 60.15 | 68.93 |

TABLE 13
Concentration of Tamapal1 in stomach, liver, intestine and kidney at
after oral administration of Tamapal1 at 0.5, 1, 2, 4, 8, 12 hours

| | Concentration of Tamapal1 (μg/ml) | | | | |
|---|---|---|---|---|---|
| Time | Stomach | Liver | Intestine | Kidney | Control |
| 30 mins | 1.0666 | 0.6305 | 0.8114 | 0.0000 | 0.0000 |
| 1 Hours | 0.9357 | 0.7289 | 0.8514 | 0.0000 | 0.0000 |
| 2 Hours | 0.7156 | 1.0873 | 0.9822 | 0.0000 | 0.0000 |
| 4 Hours | 0.3635 | 0.9620 | 1.1676 | 0.0000 | 0.0001 |
| 8 Hours | 0.3487 | 0.6425 | 0.9097 | 0.0117 | 0.0000 |
| 12 Hours | 0.3480 | 0.4738 | 0.8927 | 0.0000 | 0.0001 |

As for Tamapal1, result showed (Table 13 and FIG. 13C) that the drug was absorbed into the stomach and blood system. The concentration of Tamapal1 was detected at 30 minutes at about 0.716 μg/ml in the stomach. Then the concentration in the stomach was about 0.936 μg/ml at 1 hour, and 1.066 μg/ml at 2 hours. Tamapal1 was then released into the blood system at 1-2 hours, and the concentration peaked at 1-2 hours at 1.45 μg/ml and 1.03 μg/m1 respectively. Tamapal1 begun to increase in the liver from 2 to 4 hours and was detected to be 1.087 μg/ml and 0.942 μg/ml. In the intestine, Tamapal1 started to peak from 8 and 12 hours at 0.982 μg/ml and 1.17 μg/ml respectively. Tamapal1 was also detected in the kidney at 0.0117 μg/ml at 8 hours and at 12 hours Tamapal1 was not detected.

### Example 13

### Thermostability Trials on Various Drugs

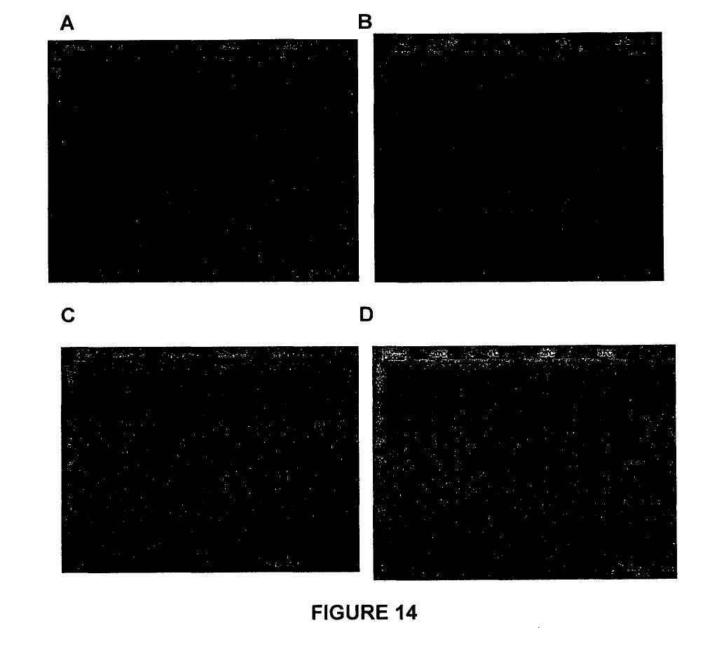

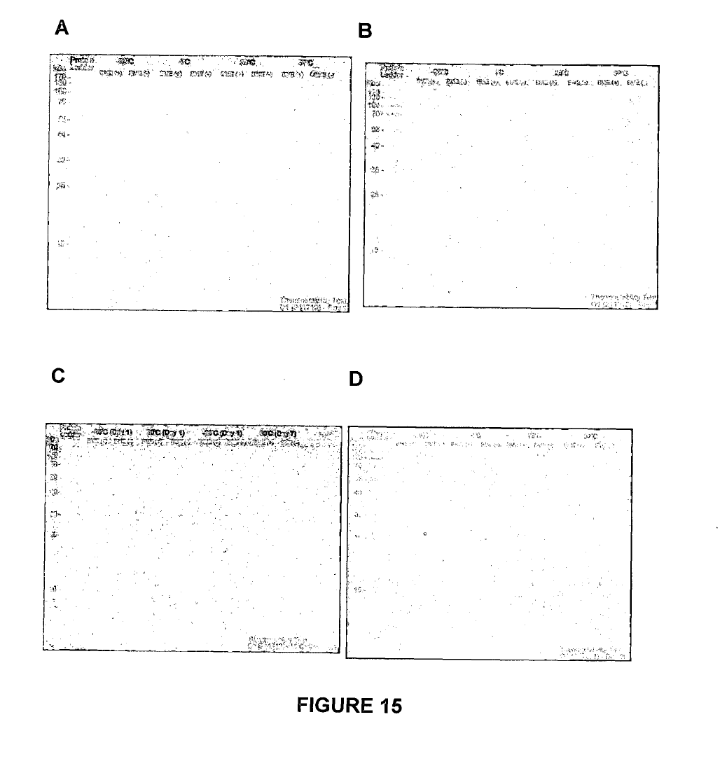

Thermostability trials as disclosed in Example 6 are carried out for the other drugs—RetroGAD1 and Tamapal1. The protein drugs RetroGAD1 and Tamapal1 are incubated at −20° C., 4° C., 26° C., 37° C. and 50° C. for different time points (1 day, 7 days and 30 days). The structural nature of protein drugs was then determined by SDS-page with the comparison to the control (protein drugs are incubated in −20° C.). The results are shown in FIGS. 14 and 15 respectively.

### Example 14

### The Antiviral Activity of RetroGAD1, RetroMAD1 and Tamapal1 Against Herpes Simplex Virus Type 2 (HSV-2), a Potential Oncogenic Virus

The effect of RetroGAD1, RetroMAD1 and Tamapal1 on the growth of Vero cells was examined to rule out any direct cytotoxicity. Monolayer cultures of Vero cells were exposed to increasing concentrations of RetroGAD1, RetroMAD1 and Tamapal1. The experimental protocol described in Example 8 was followed. After 24, 48 and 72 hours of incubation, cell viability was determined using MTS assay as described in Example 9. Results obtained showed that the accepted maximal nontoxic dose (MNTD) of RetroGAD1 and Tamapal1 was 10 μg/ml. For RetroMAD1 the MNTD was 50 μg/ml. At the chosen MNTD, the peptides did not impair the cell viability with respect to the untreated control group.

The antiviral activity of RetroGAD1, RetroMAD1 and Tamapal1 was evaluated by simultaneous treatment. For simultaneous treatment the mixture of the respective peptide and virus was inoculated onto Vero cells and incubated for 24, 48 and 72 hours at 37° C. under 5% CO₂ atmosphere. At the end of the time period the samples were harvested and viral DNA was extracted. The eluted DNA was then subjected to RT-PCR.

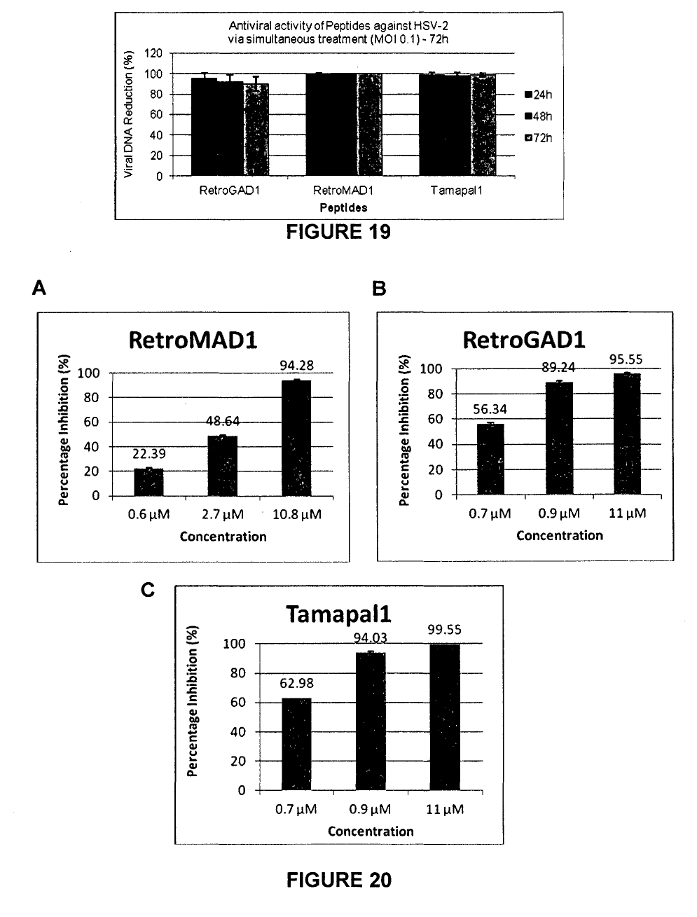

The results obtained suggested that all the three peptides have strong inhibitory activity against HSV-2 via simultaneous treatment at the maximal non-toxic dose (MNTD). RetroGAD1 exhibited 95.45, 91.71 and 89.95% inhibitory activity, respectively, at 24, 48 and 72hours (table 14 and FIG. 19). RetroMAD1 showed 99.67, 99.96 and 99.87% of viral reduction, respectively, at 24, 48 and 72 hours (Table 14 and FIG. 19). Tamapal1 showed 98.75, 98.00 and 98.98% inhibition, respectively, at 24, 48 and 72 hours (Table 14 and FIG. 19).

TABLE 14
Percentage of viral reduction by RetroGAD1, RetroMAD1 and
Tamapal1 in simultaneous treatment at 72 hours determined by PCR.

| | Time | | |
|---|---|---|---|
| Peptides | 24 h | 48 h | 72 h |
| RetroGAD1 | 95.45% | 91.71% | 89.95% |
| RetroMAD1 | 99.67% | 99.96% | 99.87% |
| Tamapal1 | 98.75% | 98.00% | 98.98% |

### Example 15

### NS2B and NS3 are Two of Seven Non-Structural Proteins Which may be Translated From the Single Open Reading Frame (ORF) in a Flavivirus RNA, and Forms the Serine Protease Complex

NS2B-NS3. It is a crucial molecule in viral replication for processing non-structural regions and therefore is an attractive target for the development of antiviral drugs or compounds. An NS2B-NS3 protease assay using fluorogenic peptides was conducted to investigate the inhibitory characteristics of the drug against the protease at various concentrations and temperatures, using the method established by Rohana et. al. (2000).

Reaction mixtures were prepared with the following reagents: 2 μM isolated NS2B-NS3 protein complex from the DENV-2 viral genome, buffer at pH 8.5 (200 mM Tris-HCl) and different concentrations of the drugs respectively. After incubation at 37° C. for 30 minutes, 100 μM fluorogenic peptide substrate was added to the mixture, which was further incubated for another 30 minutes. Triplicates were performed for each concentration and readings were taken with a Tecan Infinite M200 Pro fluorescence spectrophotometer. Substrate cleavage was optimized at the emission of 440 nm upon excitation at 350 nm.

All of the drugs showed strong inhibition against this protease. Although RetroMAD1 has least inhibition activity against NS2B-NS3 compared to the other drugs, it managed to inhibit 94.28% of NS2B-NS3 at the concentration of 10.8 μM (FIG. 20A). RetroGAD1 inhibited 95.55% of NS2B-NS3 at 11 μM (FIG. 20B). Tamapal1 showed the strongest inhibition against NS2B-NS3 where more than 50% of NS2B-NS3 is inhibited by just using concentration of 0.7 μM. At 11 μM of Tamapal1 inhibition was nearly 100% of NS2B-NS3 (FIG. 20C).

### Example 16

### The Antiviral Activity of RetroMAD1 Against Dengue Viruses (DENV-1, DENV-2, DENV-3, DENV-4) Showing NS2B NS3 Inhibition was Effective

The antiviral activity of RetroMAD1 was evaluated by simultaneous treatment. For simultaneous treatment the mixture of the respective peptide and virus was inoculated onto Vero cells and incubated for 24, 48 and 72 hours at 37° C. under 5% CO₂ atmosphere as described in Examples 8 and 9 above. At the end of the incubation period the samples were harvested and viral RNA was extracted. The eluted RNA was then subjected to RT-PCR.

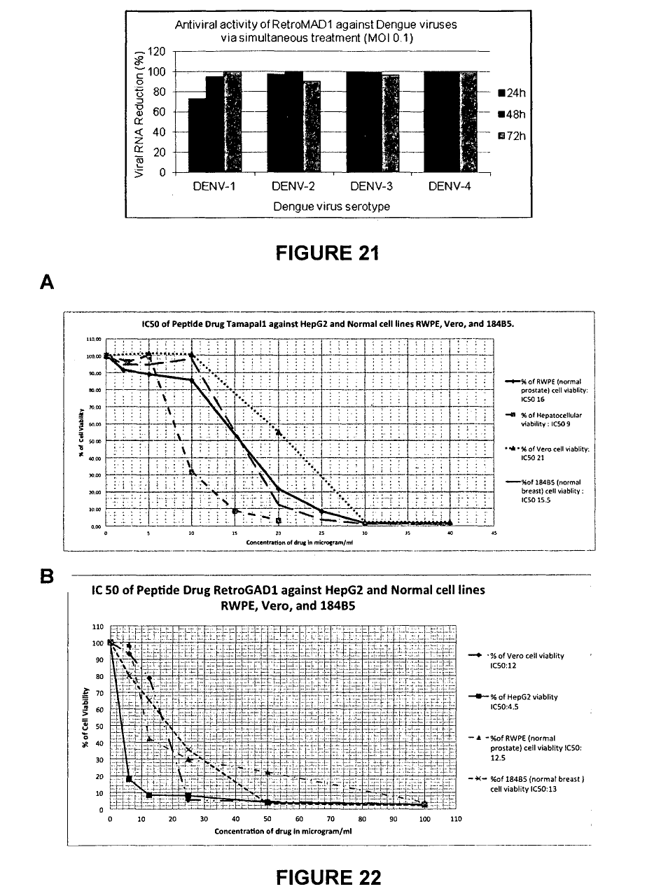

The results obtained suggested that RetroMAD1 had a strong inhibitory activity on all four Dengue virus serotypes (DENV-1, DENV-2, DENV-3 and DENV-4) via simultaneous treatment at the maximal non-toxic dose (MNTD), 50 μg/ml of RetroMAD1 exhibited 99.50, 89.80. 96.15 and 99.90% inhibitory activity, respectively, against DENV-1, DENV-2, DENV-3 and DENV-4 at 72 hours (Table 15 and FIG. 21).

TABLE 15
Percentage of viral reduction by RetroGAD1, RetroMAD1 and
Tamapal1 in simultaneous treatment at 72 h determined by PCR.

| | Time | | |
|---|---|---|---|
| Peptides | 24 h | 48 h | 72 h |
| DENV-1 | 72.60 | 94.85 | 99.50 |
| DENV-2 | 96.65 | 99.45 | 89.80 |
| DENV-3 | 99.80 | 98.75 | 96.15 |
| DENV-4 | 100.00 | 100.00 | 99.90 |

### Example 17

### Activity of Tamapal1 and RetroGAD1 against HepG2 Liver Cancer Cell Line vs. Normal Cell Culture and Treatment

The HepG2 and Vero cells were purchased and cultured in Dulbecco's Modified Eagle Medium (DMEM) (HyClone) containing 10% Fetal bovine serum (FBS) (HyClone). The flask was placed in an incubator at 37° C. to allow virus adsorption. The normal cells RWPE was grown in KBM-CD (Lonza) and PC3 grown in RPMI 1640 (Lonza)

### In Vitro Determination of Anti-Cancer Activity

All the cells were grown in standard cell medium (DMEM) supplemented with 10% fetal bovine serum in a 5% CO2 atmosphere. The cells were then transferred into 96 well plate at the concentration of 1×10⁴ cells per well for cytotoxicity test. The cells were treated with our candidate drugs Tamapal1 and RetroGAD1.

The in vitro cytotoxicity analysis was carried out on our candidate drugs to determine the IC50 to all cell lines used in this experiment. The concentrated stock of drugs was diluted with respective media (depending on the cell line used) before adding to a pre-plated monolayer of cells in 96-well plates. A series of suitable controls for in vitro determination was included in every plate and the plates are incubated in the optimal conditions.

At 24 h of incubation, proliferation was measured by the colorimetric MTS (Promega CeilTiter 96® AQueous Non-Radioactive Cell Proliferation Assay (Promega, USA) according to the manufacturer's protocol (Malich et al., 1997) assay.

The assay was carried out as per manufacture's instruction. The half maximal inhibitory concentration (IC50) value was calculated using the formula:

$$Percentage of cell viability \text{:} \frac{[ Mean O D of the test group - Mean O D of the blank group ]}{[ \begin{matrix}Mean O D of the control group - \\ Mean O D of the blank group\end{matrix} ]} \times 100 \%$$

Tamapal1 was shown to have anticancer activity against Prostate cancer PC3 and Hepatocellular Carcinoma HepG2. When tested against an array of normal cell lines for eg. Vero, RWPE and 184B5. IC 50 results showed one and a half to four times increase when compared to the normal cell lines (FIG. 22A). This shows that the drug killed the cancer cell and did not affect the normal cell lines.

RetroGAD1 greatly contributed to anticancer activity in present study. IC50 4.5 to 6 μg/ml against HegG2 cell line obtained in present study implied the potential use of RetroGAD1 in the Hepatocarcinoma cancer treatment. When tested against the array of normal cell lines for eg: Vero, RWPE, 184B5 and it was found that the IC50 value escalated twice when compared to our carcinoma cells (FIG. 22B).

These preliminary and onset of data implies that the drug possess high therapeutic index and non-toxic to normal cell lines. These results show the selective nature of the drugs and would help to future quantify the relative safety of the drugs.

### Example 18

### Activity of Tamapal1 Against Prostate Cancer Cell Line

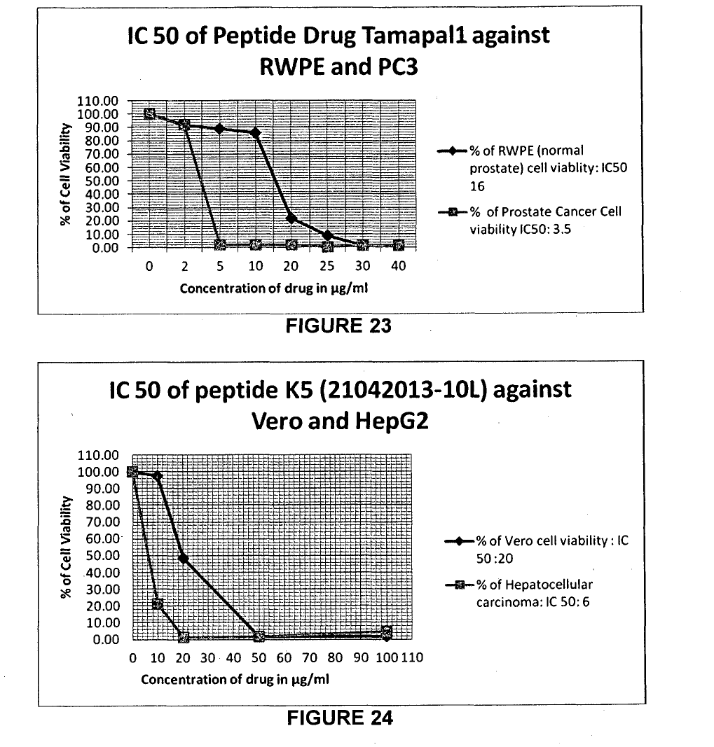

The same example as that in Example 17 was carried out with prostate cancer cell line (PC3) and Tamapal1. There was a large therapeutic index of 4 obtained when PC3 was tested with the normal prostate cells (RWPE) (FIG. 23). Therefore when prostate cancer cells were treated with Tamapal1, the normal cells remain unaffected.

### Example 19

### K5 Activity Against HepG2 Compared with Vero Cells

The same example as that in Example 17 was carried out with HepG2 and K5 peptide. The peptide drug K5 has a therapeutic index of 3.8 (FIG. 24). Hence, showing that K5 targeted the cancerous cell and not the normal cell at low concentration.

### Example 20

### Drug Mechanism Using the Merck Millipore MUSE Platform Caspase Activity

There are a number of caspases in mammalian cells that have been shown to be involved in the early stages of apoptosis, e.g. Caspase 2, Caspase 3, Caspase 6, Caspase 7, Caspase 8, Caspase 9 and Caspase 10. The functions of these enzymes are not yet entirely clear, but it appears that after an initial signal to the cell to undergo apoptosis, they may be responsible for the activation, amplification and execution of the apoptotic cascade. Because of the central importance of the caspases in apoptosis, their detection by flow cytometry was carried out using the MUSE platform.

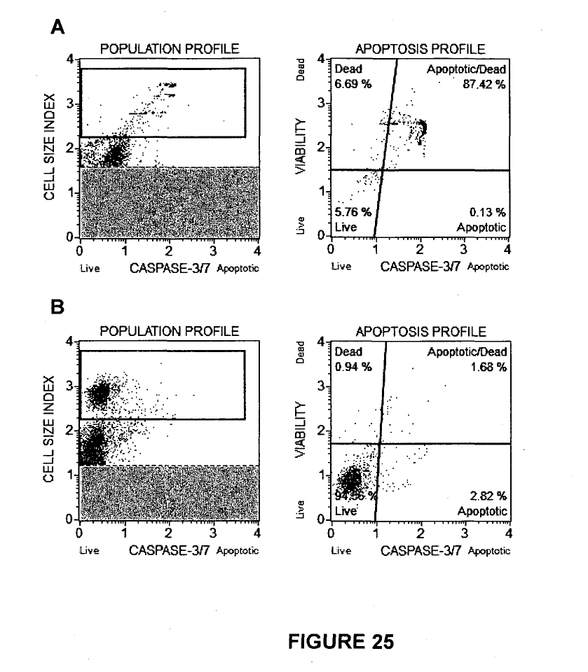

The drugs were tested against HepG2 using Muse Kits for caspase. The Kits for caspase were purchased from Muse™ Caspase-3/7 Kit, Merck Millipore. Samples were prepared for the test according to the manufacturer's instructions. The cells were stained and analysed for caspase activity. The concentrated stock of drug RetroGAD1, Tamapal1, and K5 was diluted to different concentrations with respective media (depending on the cell line used) before adding to a pre-plated monolayer of cells in plates. The results (FIGS. 25A and B) showed that when the samples were treated for different concentrations, the cells were induced for apoptosis. The assay provided relative percentage of cells that are live, in the early and late stages of apoptosis, and dead. As the concentration of RetroGAD1 was increased, the induction of apoptosis also increased gradually Table 16.

TABLE 16
Caspase activity for different drug concentration of RetroGAD1

| | % of cells |
|---|---|
| | expressing |
| Concentration of | caspase |
| RetroGAD1 in μg/ml | activity |
| 30 | 87.49 |
| 25 | 75.45 |
| 15 | 18.52 |
| 10 | 18.28 |
| 5 | 9.89 |

TABLE 17
Caspase activity for different drug concentration of Tamapal1

| | % of cells |
|---|---|
| | expressing |
| Concentration of | caspase |
| Tamapal1 in μg/ml | activity |
| 10 | 0.10 |
| 5 | 2.82 |
| 3.5 | 2.10 |
| 2 | 1.65 |

TABLE 18
Caspase activity for different drug concentration of K5

| | % of cells |
|---|---|
| | expressing |
| Concentration of K5 | caspase |
| in μg/ml | activity |
| 20 | 1.78 |
| 13 | 1.15 |
| 5 | 1.15 |

In conclusion, RetroGAD1 showed apoptotic properties by activation of cells expressing caspase activity, while Tamapal1 and K5 did not show a significant percentage of caspase activity.

### P13 Kinase Pathway

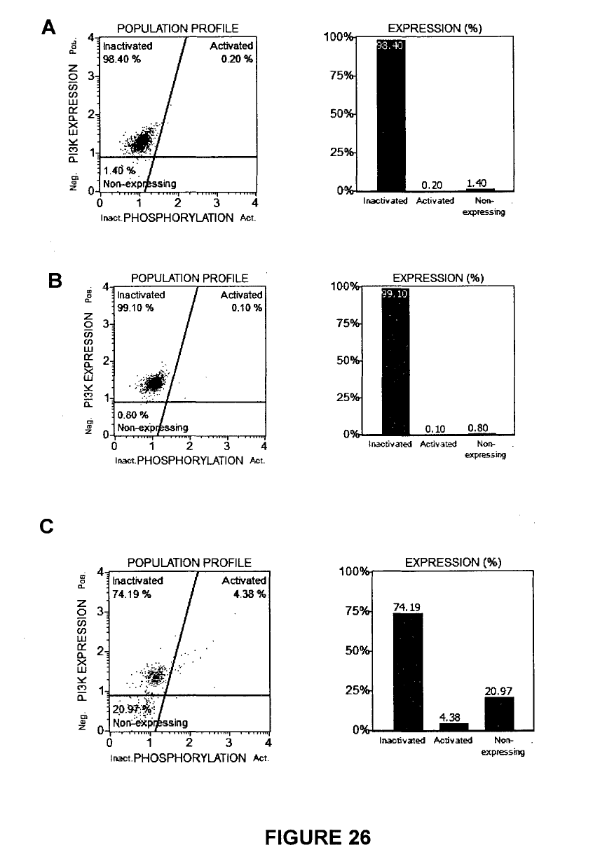

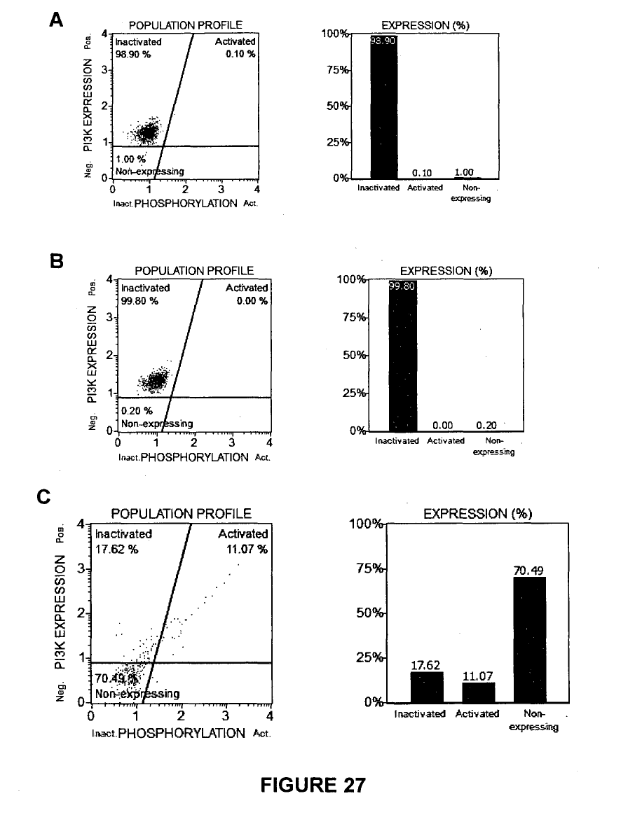

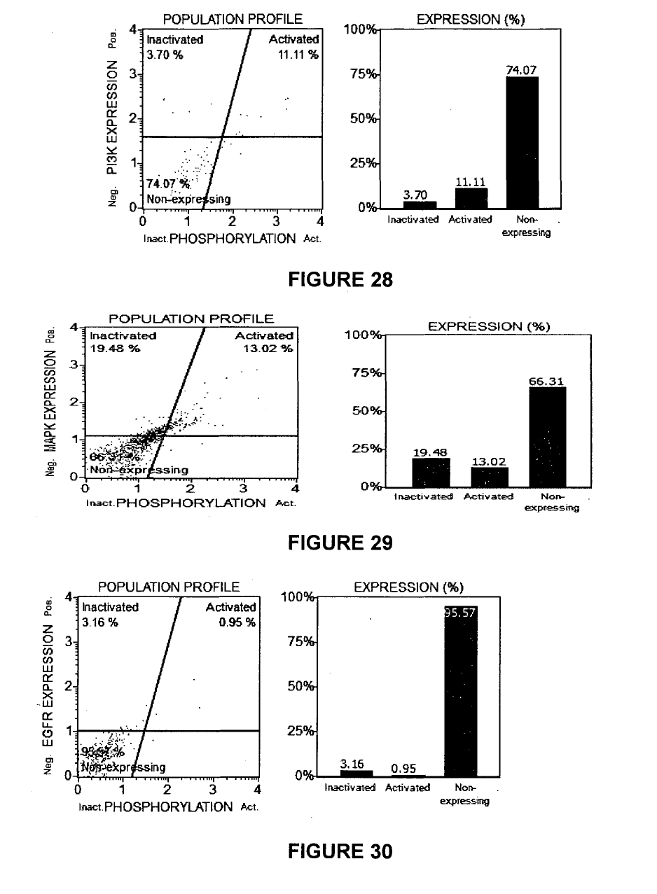

RetroGAD1, Tamapal1 and K5 were tested against HepG2 using Muse Kits for P13. The Kits for PI3 kinase were purchased from Muse™, Merck Millipore. The samples were prepared for the test according to the manufacturer's instruction. The cells were stained and analysed for P13 activity. The concentrated stock of the candidate drug was diluted to different concentrations with DMEM before adding to a pre-plated monolayer of cells in plates. The results are shown in Table 19 and FIGS. 26, 27 and 28 of the treated samples at different concentrations. Table 19 gives the results for K5 and Tamapal1, tested from lower to higher concentration. At higher concentration, the inactivation percentage decreases owing to the condition of higher toxicity to cells.

TABLE 19
The percentage of PI3 Kinase inactivation by
peptide Drug K5 and Tamapal1

| | Concentrations | % of PI3 Kinase |
|---|---|---|
| Drug | (μg/ml) | inactivation |
| K5 | 5 | 98.4 |
| | 13 | 99.10 |
| | 40 | 74.19 |
| Tamapal1 | 5 | 98.90 |
| | 15 | 99.80 |
| | 30 | 17.62 |

The results showed that Tamapal1 and K5 could inhibit or inactivate 90% of the PI3 pathway in hepatocarcinoma.

### MAPK Pathway

Flow cytometry analysis was used to study the action of RetroGAD1 in MAPK pathway inhibition in HepG2 cells. The MAPK pathway Flowcytometry kit was purchased from Muse™ MAPK Activation Dual Detection Kit, Millipore. The experiments were conducted as described by the manufacturer for different concentrations of RetroGAD1, Tamapal1 and K5. When the cell lines were treated with the drug, there was some evidence of inactivation of the MAPK pathway. However, as a relatively high concentration of drug was used (30 μg/m1) resulting in just below 20% inactivation, it may be assumed that the MAPK pathway was not significantly targeted by the drugs. One of the results is depicted below in FIG. 29.

### EGFR Pathway

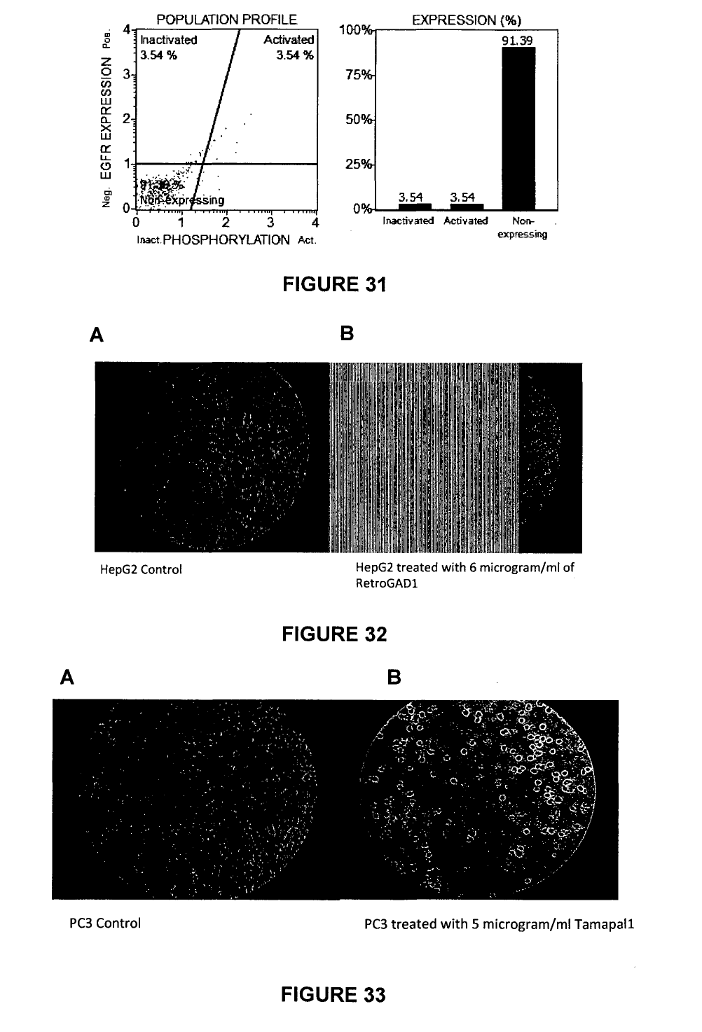

Flow cytometry analysis was used to study the action of RetroGAD1, Tamapal1 and K5 in EGFR pathway inhibition in HepG2 cells. The Flowcytometry kits for EGFR pathway was purchased from Muse™ EGFR Activation Detection Kit, Millipore. The experiments were conducted as described by the manufacturer for different concentrations of RetroGAD1. There was evidence in inactivation of the pathway which showed about 1 to 4 percent of inactivation when there was an increase in the concentration of the respective peptide drug. The low inactivation result suggested that this pathway is not targeted by the drugs. One of the results is depicted in FIGS. 30 and 31.

### Example 21

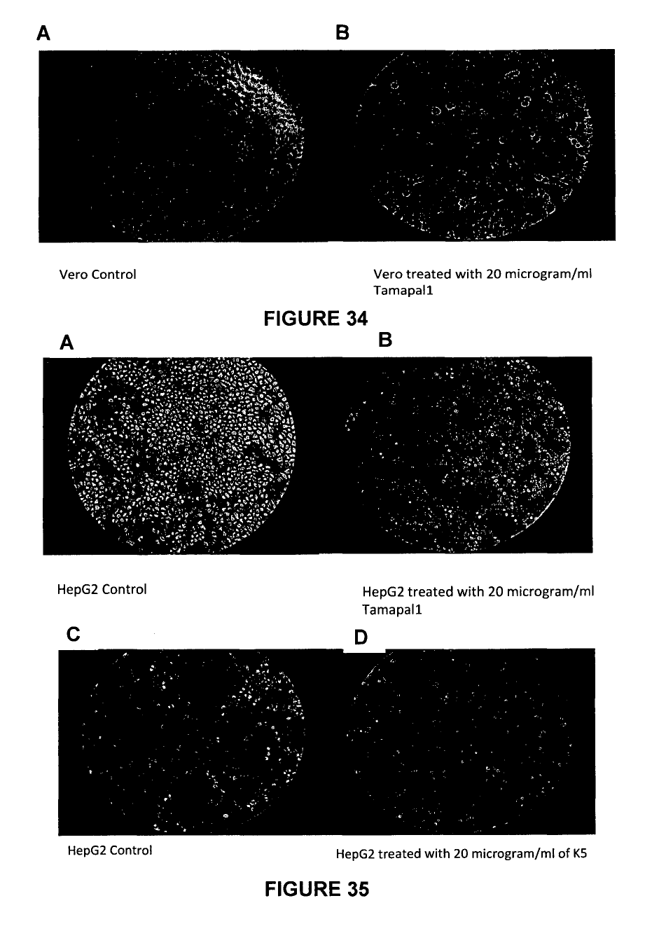

Cancer cells HepG2 and PC3 were plated onto a 96-well plate for the MTS assay done in Example 17. The cells were treated with RetroGAD1, Tamapal1 and K5 respectively at different concentrations. After 24 hours cells were view under XV-I-OPTIKA-100 microscope at 40× magnification and pictures were taken of control and treated cells. The results are showing treated and untreated cells in FIGS. 32-35.

### Example 22

### RT-PCR Microarray Results Suggesting a Potential Mechanism of Action

This assay was conducted in a 96-well plate which was pre-configured with the most appropriate TaqMan® Gene Expression Assay for a specific pathway in cancer. The panel of assays in the TaqMan® Array 96-well Human Apoptosis Plate targeted genes from both of the signaling pathways that initiate mammalian apoptosis, the death receptor regulated pathway and the BCL-2 family pathway. Genes such as caspases which are involved in the final mechanisms of both cell death pathways are also present in the panel.

The PCR array is a set of optimized real-time PCR primer assays on 96-well which focuses on apoptosis profile in cancer pathway. The RNA were harvested from HepG2 cells treated with Tamapal1 with its IC50 value using RNAqueous®-4PCR Kit by Applied Biosystems and converted to cDNA using High Capacity RNA to cDNA Kits by Applied Biosystems according to the manufacturer's instruction. The final samples were aliquoted and RT PCR was performed to study the gene expression and the multi-gene profiling capability of a microarray.

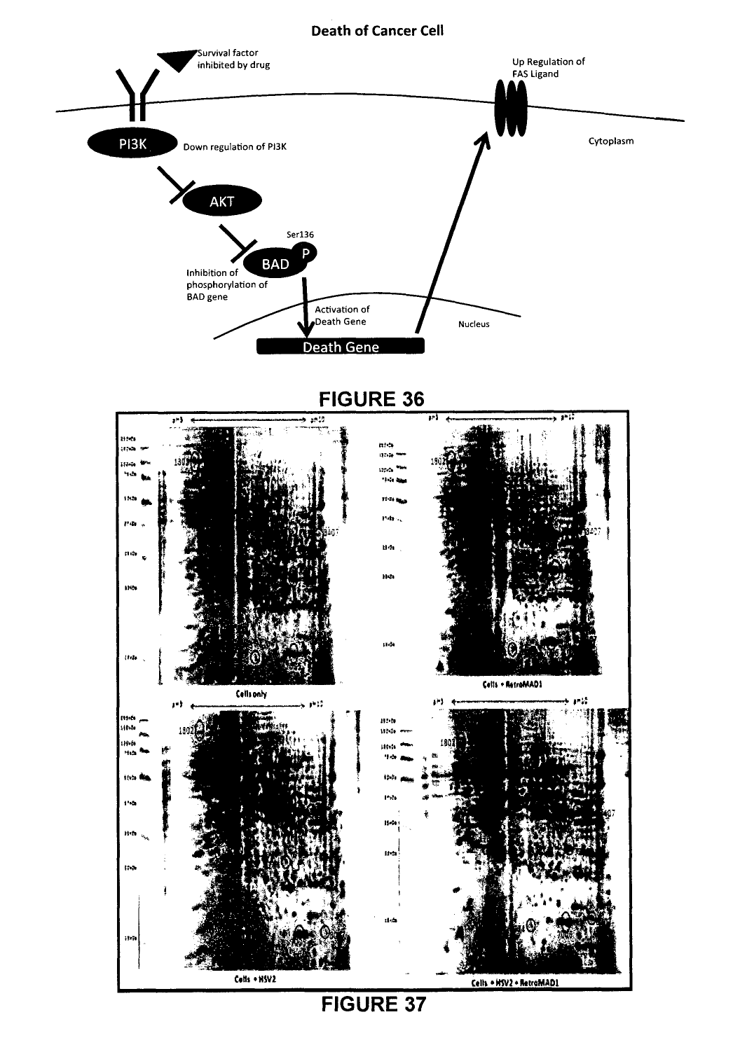

The results are provided in Table 20 and the possible role of the fusion protein (Tamapal1) given in FIG. 36. The flow cytometry results showed that the PI3 kinase pathway was down regulated by Tamapal1 and K5 in HepG2 cell lines.

One of the consequences of PI3K or AKT activation is engagement of an anti apoptotic pathway. This involves a variety of substrates downstream of AKT that are inhibited or activated to prevent apoptosis. For example, AKT prevents release of cytochrome c from mitochondria and inactivate forehead (FKHR). AKT phosphorylates and inactivates a pro-death protease, caspase 9, and the anti-apoptotic factor BAD. AKT via IKK induces nuclear translocation of the survival protein NF-KB AND MDM2 and targets the tumor suppressor gene P53 for degradation by the proteosome (Mayo LD and Donner DB 2001).

The results revealed a complex network of remarkable redundancy that connected signals from the tumour microenvironment with BAD phosphorylation, which in turn showed an up-regulated profile in the RT PCR array. So, the levels of cancer progression were ultimately reduced.

TABLE 20
The results for HepG2 when treated with Tamapal1

| | Control | | Expression of | |
|---|---|---|---|---|
| | CT | Tamapal1 | genes (compare | |
| Genes | value | CT value | to the control) | Function |
| Bcl-2-associated | 35.56 | 21.87 | Up regulated | Pro- |
| death promoter | | | | apoptotic |
| (BAD) | | | | |
| BCL2-like 13 | 15.67 | 35.07 | Down regulated | Pro- |
| (BCL2L13) | | | | apoptotic |
| Fas Ligand | 22.76 | 15.26 | Up regulated | Death |
| (FAS) | | | | receptor |
| Lymphotoxin | 32.2 | 25.03 | Up regulated | Tumour |
| Beta (LTB) | | | | necrosis |
| | | | | factor |

### Example 23

### Proteomics Analysis Indicating Pathways Involved in the Inhibition of HSV2, a Potential Oncogenic Virus

A protein profile was obtained from two dimensional gel electrophoresis and mass spectrometry analysis to study the effect of RetroMAD1 on protein expression in Herpes Simplex Virus 2 (HSV2) infected cells. 2D gel electrophoresis analysis revealed significantly altered levels of proteins expression, proteins were identified by tandem MS (MS/MS).

Equal amounts of total protein from (i) cells only, (ii) RetroMAD1 treated cells, (iii) HSV2-infected cells, and (iv) RetroMAD1 treated-infected cells, were subjected to 2D gel electrophoresis. 250 μg of proteins were rehydrated into 13 cm immobilized pH gradient (IPG) strips (pH 3-11 nonlinear) (GE Healthcare). The first dimension was electrophoresed on the IPGphor III machine (GE Healthcare) at 20° C. with the following settings: step 1 at 500V for 1 hour; step 2 at 500-1000V for 1 hour; step 3 at 1000-8000V for 2.5 hour, and step 4 at 8000V for 0.5 h. After first dimensional separation, the gel was equilibrated as follows; first reduction with 64.8 mM of dithiothreitol-SDS equilibration buffer (50 mM Tris-HCl [pH 8.8], 6 M urea, 30% glycerol, 2% SDS, and 0.002% bromophenol blue) for 15 minutes, followed by alkylation with 135.2 mM of iodoacetamide-SDS equilibration buffer for another 15 minutes. The second dimension electrophoresis was carried out using the SE600 Ruby system (GE Healthcare) at 25° C. in an electrode buffer (25 mM Tris, 192 mM glycine, and 0.1% [wt/vol] SDS) with the following settings: step 1 at 100V/gel for 45 minutes; step 2 at 300V/gel until the run is completed. After electrophoresis, the gels were fixed with destaining solution for 30 minutes, followed by staining with hot Coomasie blue for 10 minutes. The gels were scanned using Ettan DIGE Imager (GE Healthcare). Gel images were analyzed using PDQuest 2-D Analysis Software (Bio-Rad, USA) and only protein spots which showed significant differences (more than 2.0 fold) were selected for mass spectrometry analysis. Identification of proteins was performed by using Mascot sequence matching software [Matrix Science] with Uniprot database.

The HSV2 replication cycle involves: (1) viral attachment; (2) viral entry; (3) membrane fusion; (4) RNA release; (5) viral protein production; (6) RNA replication; (7) viral assembly; (8) viral transport and maturation and lastly (9) viral release. There are two important HSV viral glycoproteins, namely glycoprotein B (gB) and glycoprotein D (gD) that are essential for facilitating efficient virus entry via the interaction with the host heparan sulphate receptors and associated co-receptors. Glycoprotein B (gB) precursor is transiently associated with calnexin, a membrane-bound chaperone, in the ER that assist in viral entry. Thus, down regulation of calnexin leads to a reduction in virus entry into the cells. Proteins involved in viral RNA release and nuclear transport like Protein disulfide-isomerase (PDI) was upregulated in RetroMAD1 treated cells. PDI has been demonstrated to play a role in redox control at the cell surface. In response to increased extracellular reduction, PDI may help to re-establish redox homeostasis by rearranging and forming disulfide bonds, thereby protecting the cell against this aggression. The viral replication and the increased expression of the viral proteins as well as the introduction of the RetroMAD1 induced cellular stress to the host cell and triggered the increased expression of the heat shock protein 70 kDa and chaperone proteins including protein disulfide isomerise, superoxide dismutase and peroxiredoxin-6 to respond to the accumulation of unfolded or misfolded viral or host proteins. RetroMAD1 down regulate cofilin1, a key regulator of actin cytoskeleton dynamics that inhibit HSV-induced rearrangements of actin cytoskeleton which is important for infectivity.

Other proteins identified, Glyceraldehyde-3-phosphate dehydrogenase and Triosephosphate isomerase involved in glycolysis pathway were found to be down-regulated. Thus, decrease of energy source needed for variety of cellular processes may lead to the inhibition of replication and amplification of viral DNA and RNA. Proteins involved in viral RNA transcription and translation such as 40S ribosomal protein and Heterogeneous nuclear ribonucleo protein A1 were down regulated and lead to a decrease in viral replication in host cells. Nucleolin was found to be down regulated by RetroMAD1. UL12, an alkaline nuclease, encoded by HSV and suggested to be involved in viral DNA maturation and nuclear egress of nucleocapsids form a complex with nucleolin, a nucleolus marker, in infected cells. Knockdown of nucleolin in HSV-infected cells reduced capsid accumulation. These results indicated that nucleolin was a cellular factor required for efficient nuclear egress of HSV nucleocapsids in infected cells.

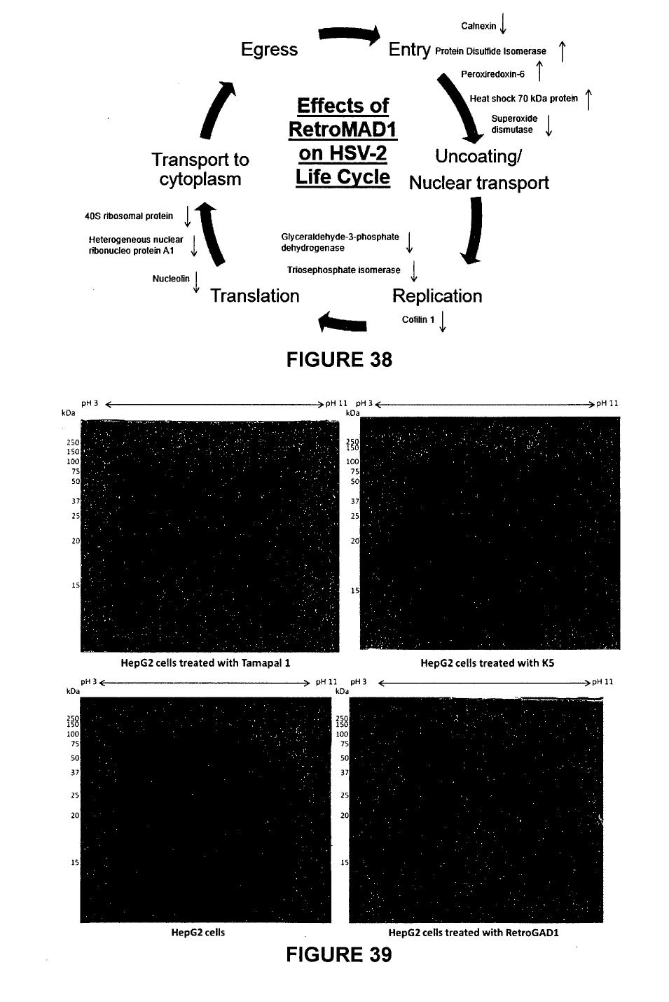

Base on the findings of this study, proteins that are differentially expressed were involved in several biological processes, including viral entry, protein folding, viral transcription and translation regulations, cytoskeletal assembly, and cellular metabolisms. This indicates that antiviral activities of RetroMAD1 could act on various action on the virus infection pathways, that is via blocking of viral adsorption, replication and also via virucidal effects. In conclusion, the inhibitory effect of RetroMAD1 occurred at various stages of viral life cycle and strongly suggests its potential as a broad spectrum antiviral agent. The protein profile is shown in FIG. 37 and the up/down regulation shown in Table 21. The effect of RetroMAD1 on the actual pathway is provided in FIG. 38.

TABLE 21
Fold changes of differential proteins in cells treated with RetroMAD1,
Cell infected by HSV2 and HSV2 infected cells treated with RetroMAD1.
Symbols “+” indicate upregulation and “−” indicate downregulation.

| | | | Cells + | Cells + Virus |
|---|---|---|---|---|
| | | Cells + | Virus | (HSV2) + |
| | Cells | RetroMAD1 | (HSV2) | RetroMAD1 |
| Protein Folding | | | | |
| Protein disulfide- | 0.00 | +1.87 | −5.03 | +2.03 |
| isomerase | | | | |
| Calnexin | 0.00 | −2.51 | +3.77 | −6.17 |
| Heat shock 70 kDa | 0.00 | −1.80 | −9.07 | +1.84 |
| protein | | | | |
| Energy, Transport, | | | | |
| Metabolism | | | | |
| Nucleoside diphosphate | 0.00 | +1.55 | −1.11 | +2.48 |
| kinase | | | | |
| Glyceraldehyde-3- | 0.00 | −1.27 | +2.90 | −1.24 |
| phosphate dehydrogenase | | | | |
| Triosephosphate | 0.00 | +2.60 | +2.41 | +1.47 |
| isomerase | | | | |
| Oxidative Proteins | | | | |
| Superoxide dismutase | 0.00 | +1.14 | −3.38 | +3.52 |
| Peroxiredoxin-6 | 0.00 | +1.82 | −1.30 | +1.98 |
| Transcription/Translation | | | | |
| 40S ribosomal protein | 0.00 | +4.7 | +2.78 | −1.73 |
| Heterogeneous nuclear | 0.00 | −1.07 | −2.14 | −1.08 |
| ribonucleo protein A1 | | | | |
| Nucleolin | 0.00 | −1.55 | −10.04 | +17.89 |
| Cytoskeleton | | | | |
| Cofilin-1 | 0.00 | +1.01 | +2.94 | +1.27 |

### Example 24

### Proteomics Analysis Showing Mechanisms Against Cell Proliferation in HepG2 Treated with RetroGAD1, Tamapal1 and K5

The same experiment as Example 23 was carried out using RetroGAD1, Tamapal1 and K5

TABLE 22
Fold changes of differential proteins in HepG2 cells treated with RetroGAD1, Tamapal1 and K5.
Symbols “+” indicate upregulation and “−” indicate downregulation.

| | | | | HepG2 | |
|---|---|---|---|---|---|
| | | | | cells | HepG2 |
| | | | HepG2 cells | treated | cells |
| | | | treated with | with | treated |
| Spot | Protein ID | Functions | RetroGAD1 | Tamapal1 | with K5 |
| | Chaperone | | | | |
| 2625 | heat shock 70 kDa protein 5 | protein folding, cell invasion | +1.63 | 0.00 | +3.37 |
| | (glucose-regulated protein, | and migration | | | |
| | 78 kDa) | | | | |
| 3611 | heat shock cognate 71 kDa | protein folding, cell | +6.13 | +3.18 | +6.57 |
| | protein isoform 2 | profileration, invasion and | | | |
| | | migration | | | |
| 1506 | Chain A, Human Protein | cell invasion, migration and | +2.88 | +2.46 | +3.53 |
| | Disulfide Isomerase, Nmr, 40 | adhesion | | | |
| | Structures | | | | |
| | Cellular receptor | | | | |
| 4705 | 48 kDa histamine receptor | cell proliferation, invasion | −3.34 | −6.32 | −4.17 |
| | subunit peptide 4 | and migration | | | |
| | Glycolytic enzyme | | | | |
| 4103 | ENO1 protein, partial | cell invasion and migration | −3.83 | −3.29 | −4.67 |
| 5406 | Pyruvate kinase muscle | cell proliferation | +2.35 | +2.08 | +5.49 |
| | isozyme (PKM2), partial | | | | |
| | Components of | | | | |
| | cytoskeletal filaments | | | | |
| 4703 | alpha-tubulin | cell proliferation, | −3.86 | −10.87 | −3.43 |
| | | maintenance of cell shape, | | | |
| | | cell migration and | | | |
| | | intracellular transport | | | |
| | Calcium-binding protein | | | | |
| 502 | Calreticulin precursor variant | protein folding, cell | −4.29 | −2.70 | −10.21 |
| | | proliferation, invasion, | | | |
| | | migration and adhesion | | | |
| | Phospholipid-binding | | | | |
| | protein | | | | |
| 2104 | Annexin A2 | cell invasion and migration, | −3.46 | −2.06 | −2.10 |
| | | induces proliferation of | | | |
| | | hepatocytes | | | |

Analysis of a two-dimensional (2D) gel electrophoresis and mass spectrometry identified 11 proteins which were differentially expressed in HepG2 after drug treatments with RetroGAD1, Tamapal1 and K5, compared to untreated HepG2 cell line. These results are found in Table 22. In HepG2 cells, proteins such as 1) 48 kDa histamine receptor, 2) ENO1 protein, 3) Alpha-tubulin, 4) Calreticulin, and 5) Annexin A2 are normally overexpressed. However, treatment of HepG2 cells with RetroGAD1, Tamapal1 and K5 showed down-regulation of these proteins and ultimately suppression of cancer cell activities:

### Cellular Receptor

48 kDa histamine receptors are normally over-expressed in cancer cells contributing to cancer cell proliferation. Upon treatment with RetroGAD1, Tamapal1 and K5, expression of histamine receptors in HepG2 cells was down-regulated, consequently inducing cell apoptosis and reducing cancer cell growth.

### Glycolytic Enzyme

Enolase 1 (ENO1) proteins are glycolytic enzymes that are highly expressed in cancer cells, which facilitate cell invasion and migration. These results showed that ENO1 protein expression in HepG2 cells were down-regulated by RetroGAD1, Tamapal1 and K5. Impairment of the glycolytic pathway results in reduction of cell proliferation and inhibition of cell invasion and migration in cancers.

### Components of Cytoskeletal Filaments

Alpha-tubulins are components of microtubules that are essential for the formation of mitotic spindles and cytoskeleton in cells, which play roles in cell migration, intracellular transport and mitosis. Expression of alpha-tubulin was down-regulated by RetroGAD1, Tamapal1 and K5, suggesting that cell migration and proliferation in cancers might be inhibited.

### Calcium-Binding Protein

Calreticulin is an intracellular calcium binding protein and it is normally over-expressed in cancer cells. Overexpression of calreticulin in cancer cells promote cell invasion and migration. Expression of calreticulin was down-regulated by RetroGAD1, Tamapal1 and K5, ultimately inhibiting cancer cell invasion and migration.

### Phospholipid-Binding Protein

Annexin A2 is a calcium-dependent, phospholipid-binding protein that is over-expressed in cancer cells. Up-regulation of annexin A2 contributes to cell proliferation, invasion, migration and adhesion in cancer cells via binding to its protein partner. Down-regulation of annexin A2 by RetroGAD1, Tamapal1 and K5 reduces the binding of annexin A2 binding to its protein partner, hence preventing cell invasion and migration.

Some proteins were shown to be up-regulated by RetroGAD1, Tamapal1 and K5 in HepG2, such as Pyruvate kinase muscle isozyme (PKM2), Protein disulfide isomerase (PDI), Heat shock cognate 71 kDa (HSC70), and Heat shock 70 kDa protein 5 (glucose-regulated protein 78 kDa).

### Glycolytic Enzyme

Pyruvate kinase muscle isozyme (PKM2) are glycolytic enzymes which are up-regulated by RetroGAD1, Tamapal1 and K5 in HepG2 cells, compared with untreated cells. PKM2 exists in two forms: tetramer (active form) and dimer (inactive form). Cancer cells over-expressed PKM2 in an inactive dimeric form to keep the rate of glycolysis low, resulting in accumulation of metabolic intermediates for the synthesis of precursor substances, such as nucleotides, amino acids, and lipids which are the material basis for cell proliferation (Wu & Le, 2013). Expression of PKM2 in HepG2 cells was greatly induced by RetroGAD1, Tamapal1 and K5 compared to untreated cells. By increasing the concentration of PKM2, it resulted in increasing the rate of tetrameric PKM2 formation, which overrode the inactive dimeric PKM2, resulting in suppression of cell proliferation since all precursor substances are being used-up.

### Chaperone

The expression of protein disulfide isomerase (PDI) was up-regulated by RetroGAD1, Tamapal1 and K5 in HepG2 cancer cell line, suggesting that overexpressing PDI in cancer cells may induce cell death in cancer. Up-regulation of PDI results in competitive inhibition of Fe³⁺ driven sequestration of caspase-3, hence promoting apoptosis (Sliskovic & Mutus, 2006). Overexpression of PDI in tumour cells suppressed the HIF-1α-regulated gene, which is the transcription activator of VEGF via interaction with Ref-1. Overexpression of PDI results in a redox state favouring the formation of a disulfide bond which stops Ref-1 activity. Inactivated Ref-1 affects HIF-1α transcription activity of VEGF, hence inhibits cancer cell growth (Hashimoto & Imaoka, 2013).

Secreted heat shock cognate 71 kDa proteins (HSC70) have recently been identified as growth arrest signals in inhibiting cancer cell growth (Nirde et al., 2010). Therefore, RetroGAD1, Tamapal1 and K5 inhibit cancer cell proliferation by inducing high expression of HSC70 in cells. Wei et al., (2012) reported that overexpression of HSP70, also known as GRP78 suppresses cancer migration in skHep-1 cells. Down regulation of GRP78 has been correlated with up regulation of vimentin, an epithelial-mesenchymal transition (EMT) marker (Tai et al, 2012) and promotes cell migration. RetroGAD1 and K5 may thus inhibit cancer cell migration through upregulation of GRP78, which plays a role in suppressing cancer cell invasion and migration.

### Example 25

### Acute Toxicity Testing in ICR Mice for Various Drugs

The acute toxicity study was used to determine a safe dose for RetroMAD1, RetroGAD1 and Tamapal1.

Adult male and female Sprague-Dawley rats (weighing about 200 g±20) were used for the trial. Rats were divided into 3 groups: control, low dose and high dose. Mice were six weeks old. The experimental protocol is provided in Table 34 below.

TABLE 23
Experimental protocol for Example 21 *BW = Body Weight

| | RetroMAD1 | RetroGAD1 | Tamapal1 |
|---|---|---|---|
| Groups | | Control, Low Dose, High Dose | |
| Female rats per | 6 (4 for control) | 4 | 4 |
| group | | | |
| Male rats per group | 6 | 4 | 4 |
| Dosing: Control | Distilled water | | |
| Dosing: Low Dose | 20 mg/kg BW | 5 mg/kg BW | 10 mg/kg BW |
| | (4 mg/ml/ | (1 mg/2 ml/ | (2 mg/2 ml/ |
| | 200 g rat) | 200 g rat) | 200 g rat) |
| Dosing: High Dose | 100 mg/kg BW | 15 mg/kg BW | 40 mg/kg BW |
| | (20 mg/4 ml/ | (3 mg/5.1 ml/ | (8 mg/5 ml/ |
| | 200 g rat) | 200 g rat) | 200 g rat) |

The test animals were fasted overnight (Day 0) prior to dosing on Day 1. The animals were given standard rat pellets and normal saline. Food was withheld for a further 3 to 4 hours after dosing. The animals were observed over a period of 2 weeks for mortality. The animals were fasted on day 14 and sacrificed on day 15 by the use of Ketamine anesthesia. Hematological and serum biochemical parameters were determined following standard methods (Tietz et al., 1983).

The study was approved by the ethics committee for animal experimentation, Faculty of Medicine, university of Malaya, Malaysia. The study was conducted in the Faculty of Medicine, university of Malaya, Malaysia. All animals received human care according to the criteria outlined in the “Guide for the Care and Use of laboratory Animals” prepared by the National Academy of Sciences and published by the National Institute of Health.

RetroMAD1 was fed at much higher doses (4 mg and 20 mg/200 g rat) compared to Tamapal1 (2 mg and 8 mg/200 g rat) while the lowest doses were that of RetroGAD1 (1 mg and 3 mg/200 g rat). The readings obtained for both the male and female fed groups were compared against their respective unfed controls and readings falling outside of the upper and lower limits of the standard deviation of the controls were interpreted as significant to be addressed. All animals survived the trials and no mortalities or abnormal behavior was observed.

TABLE 24
Hematology report for RetroMAD1, RetroGAD1 and Tamapal1 where F is female;
M is male; C is control; LD is low dose; and HD is high dose.

| RBC ×10¹²/ | Hb | PCV | MCV | MCHC | WBC | | B Neut | | S Neut | |
|---|---|---|---|---|---|---|---|---|---|---|
| L | g/L | L/L | fL | g/L | ×10⁹/L | % | ×10⁹/L | % | ×10⁹/L | % |

RetroMAD1

| | | | | | | | | | | | | |
|---|---|---|---|---|---|---|---|---|---|---|---|---|
| F-C | Mean | 7.31 | 148.00 | 0.43 | 59.32 | 341.85 | 8.56 | 1.25 | 0.12 | 10.75 | 0.99 | 80.00 |
| | SD | 0.47 | 10.92 | 0.02 | 3.19 | 13.38 | 3.40 | 0.50 | 0.09 | 3.59 | 0.72 | 4.83 |
| F-LD | Mean | 7.10 | 146.67 | 0.45 | 63.47 | 325.96 | 10.40 | 2.17 | 0.22 | 14.17 | 1.47 | 76.17 |
| | SD | 0.19 | 6.89 | 0.02 | 3.70 | 7.33 | 1.05 | 0.41 | 0.04 | 3.49 | 0.35 | 2.79 |
| F-HD | Mean | 7.43 | 143.50 | 0.44 | 59.47 | 325.00 | 10.42 | 2.50 | 0.27 | 17.17 | 1.84 | 72.33 |
| | SD | 0.33 | 3.89 | 0.01 | 1.58 | 5.67 | 2.33 | 0.55 | 0.11 | 3.49 | 0.71 | 3.72 |
| M-C | Mean | 7.34 | 153.17 | 0.46 | 62.98 | 331.92 | 9.63 | 1.50 | 0.15 | 12.33 | 1.19 | 79.83 |
| | SD | 0.41 | 3.43 | 0.01 | 2.85 | 7.91 | 3.09 | 0.55 | 0.08 | 2.16 | 0.41 | 3.66 |
| M-LD | Mean | 7.39 | 148.50 | 0.48 | 64.64 | 311.45 | 11.16 | 2.17 | 0.24 | 14.83 | 1.59 | 75.83 |
| | SD | 0.33 | 9.27 | 0.01 | 3.18 | 17.08 | 2.69 | 0.41 | 0.08 | 3.31 | 0.23 | 3.06 |
| M-HD | Mean | 7.23 | 146.17 | 0.46 | 63.74 | 317.80 | 9.67 | 2.00 | 0.19 | 15.33 | 1.45 | 74.67 |
| | SD | 0.42 | 7.41 | 0.02 | 2.68 | 5.49 | 3.08 | 0.00 | 0.06 | 4.13 | 0.49 | 3.44 |

RetroGAD1

| | | | | | | | | | | | | |
|---|---|---|---|---|---|---|---|---|---|---|---|---|
| F-C | Mean | 7.56 | 151.25 | 0.49 | 64.87 | 308.81 | 11.71 | 2.00 | 0.23 | 20.00 | 2.22 | 71.00 |
| | SD | 0.08 | 3.86 | 0.02 | 2.60 | 6.21 | 4.33 | 0.00 | 0.09 | 4.69 | 0.56 | 5.29 |
| F-LD | Mean | 7.46 | 143.75 | 0.46 | 61.72 | 312.74 | 5.00 | 1.50 | 0.07 | 17.75 | 0.85 | 73.75 |
| | SD | 1.06 | 17.95 | 0.06 | 1.03 | 2.58 | 1.43 | 0.58 | 0.03 | 4.86 | 0.14 | 4.72 |
| F-HD | Mean | 7.53 | 142.50 | 0.47 | 62.21 | 305.36 | 5.18 | 1.75 | 0.09 | 18.00 | 0.93 | 72.25 |
| | SD | 0.28 | 4.51 | 0.02 | 3.95 | 19.61 | 0.30 | 0.50 | 0.02 | 3.74 | 0.18 | 4.57 |
| M-C | Mean | 7.54 | 146.25 | 0.48 | 64.19 | 303.31 | 9.98 | 2.00 | 0.20 | 23.00 | 2.28 | 67.00 |
| | SD | 0.64 | 8.18 | 0.03 | 4.21 | 4.32 | 2.35 | 0.82 | 0.08 | 7.26 | 0.81 | 4.69 |
| M-LD | Mean | 7.04 | 137.00 | 0.45 | 63.38 | 307.73 | 6.62 | 2.50 | 0.17 | 22.25 | 1.47 | 67.50 |
| | SD | 0.63 | 10.23 | 0.03 | 2.82 | 5.19 | 0.60 | 0.58 | 0.05 | 3.10 | 0.20 | 2.89 |
| M-HD | Mean | 7.42 | 148.75 | 0.49 | 66.24 | 303.57 | 6.79 | 2.00 | 0.14 | 17.50 | 1.19 | 73.00 |
| | SD | 0.45 | 1.50 | 0.00 | 3.88 | 3.06 | 0.90 | 0.82 | 0.06 | 0.58 | 0.18 | 2.16 |

TAMAPAL1

| | | | | | | | | | | | | |
|---|---|---|---|---|---|---|---|---|---|---|---|---|
| F-C | Mean | 7.56 | 151.25 | 0.49 | 64.87 | 308.81 | 11.71 | 2.00 | 0.23 | 20.00 | 2.22 | 71.00 |
| | SD | 0.08 | 3.86 | 0.02 | 2.60 | 6.21 | 4.33 | 0.00 | 0.09 | 4.69 | 0.56 | 5.29 |
| F-LD | Mean | 7.23 | 141.00 | 0.46 | 63.02 | 310.48 | 6.74 | 2.00 | 0.15 | 18.50 | 1.28 | 72.50 |
| | SD | 0.38 | 5.03 | 0.02 | 2.80 | 18.49 | 2.10 | 0.82 | 0.10 | 5.32 | 0.58 | 4.20 |
| F-HD | Mean | 7.51 | 141.25 | 0.45 | 59.92 | 316.65 | 5.91 | 2.00 | 0.12 | 18.25 | 1.07 | 72.50 |
| | SD | 0.54 | 11.44 | 0.03 | 6.78 | 34.68 | 0.70 | 0.00 | 0.01 | 0.96 | 0.09 | 2.08 |
| M-C | Mean | 7.54 | 146.25 | 0.48 | 64.19 | 303.31 | 9.98 | 2.00 | 0.20 | 23.00 | 2.28 | 67.00 |
| | SD | 0.64 | 8.18 | 0.03 | 4.21 | 4.32 | 2.35 | 0.82 | 0.08 | 7.26 | 0.81 | 4.69 |
| M-LD | Mean | 7.58 | 151.25 | 0.49 | 64.43 | 310.21 | 9.19 | 2.00 | 0.18 | 25.75 | 2.36 | 66.00 |
| | SD | 0.40 | 7.23 | 0.01 | 2.71 | 10.72 | 0.67 | 0.00 | 0.01 | 6.85 | 0.67 | 7.62 |
| M-HD | Mean | 7.20 | 142.50 | 0.46 | 64.29 | 307.93 | 8.62 | 2.25 | 0.18 | 24.00 | 2.05 | 67.00 |
| | SD | 1.03 | 20.57 | 0.06 | 3.54 | 5.48 | 3.47 | 0.50 | 0.05 | 3.16 | 0.82 | 4.24 |

RetroMAD1

| | | | | | | | | | | | | |
|---|---|---|---|---|---|---|---|---|---|---|---|---|
| F-C | Mean | 7.31 | 148.00 | 0.43 | 59.32 | 341.85 | 8.56 | 1.25 | 0.12 | 10.75 | 0.99 | 80.00 |
| | SD | 0.47 | 10.92 | 0.02 | 3.19 | 13.38 | 3.40 | 0.50 | 0.09 | 3.59 | 0.72 | 4.83 |
| F-LD | Mean | 7.10 | 146.67 | 0.45 | 63.47 | 325.96 | 10.40 | 2.17 | 0.22 | 14.17 | 1.47 | 76.17 |
| | SD | 0.19 | 6.89 | 0.02 | 3.70 | 7.33 | 1.05 | 0.41 | 0.04 | 3.49 | 0.35 | 2.79 |
| F-HD | Mean | 7.43 | 143.50 | 0.44 | 59.47 | 325.00 | 10.42 | 2.50 | 0.27 | 17.17 | 1.84 | 72.33 |
| | SD | 0.33 | 3.89 | 0.01 | 1.58 | 5.67 | 2.33 | 0.55 | 0.11 | 3.49 | 0.71 | 3.72 |
| M-C | Mean | 7.34 | 153.17 | 0.46 | 62.98 | 331.92 | 9.63 | 1.50 | 0.15 | 12.33 | 1.19 | 79.83 |
| | SD | 0.41 | 3.43 | 0.01 | 2.85 | 7.91 | 3.09 | 0.55 | 0.08 | 2.16 | 0.41 | 3.66 |
| M-LD | Mean | 7.39 | 148.50 | 0.48 | 64.64 | 311.45 | 11.16 | 2.17 | 0.24 | 14.83 | 1.59 | 75.83 |
| | SD | 0.33 | 9.27 | 0.01 | 3.18 | 17.08 | 2.69 | 0.41 | 0.08 | 3.31 | 0.23 | 3.06 |
| M-HD | Mean | 7.23 | 146.17 | 0.46 | 63.74 | 317.80 | 9.67 | 2.00 | 0.19 | 15.33 | 1.45 | 74.67 |
| | SD | 0.42 | 7.41 | 0.02 | 2.68 | 5.49 | 3.08 | 0.00 | 0.06 | 4.13 | 0.49 | 3.44 |

RetroGAD1

| | | | | | | | | | | | | |
|---|---|---|---|---|---|---|---|---|---|---|---|---|
| F-C | Mean | 7.56 | 151.25 | 0.49 | 64.87 | 308.81 | 11.71 | 2.00 | 0.23 | 20.00 | 2.22 | 71.00 |
| | SD | 0.08 | 3.86 | 0.02 | 2.60 | 6.21 | 4.33 | 0.00 | 0.09 | 4.69 | 0.56 | 5.29 |
| F-LD | Mean | 7.46 | 143.75 | 0.46 | 61.72 | 312.74 | 5.00 | 1.50 | 0.07 | 17.75 | 0.85 | 73.75 |
| | SD | 1.06 | 17.95 | 0.06 | 1.03 | 2.58 | 1.43 | 0.58 | 0.03 | 4.86 | 0.14 | 4.72 |
| F-HD | Mean | 7.53 | 142.50 | 0.47 | 62.21 | 305.36 | 5.18 | 1.75 | 0.09 | 18.00 | 0.93 | 72.25 |
| | SD | 0.28 | 4.51 | 0.02 | 3.95 | 19.61 | 0.30 | 0.50 | 0.02 | 3.74 | 0.18 | 4.57 |
| M-C | Mean | 7.54 | 146.25 | 0.48 | 64.19 | 303.31 | 9.98 | 2.00 | 0.20 | 23.00 | 2.28 | 67.00 |
| | SD | 0.64 | 8.18 | 0.03 | 4.21 | 4.32 | 2.35 | 0.82 | 0.08 | 7.26 | 0.81 | 4.69 |
| M-LD | Mean | 7.04 | 137.00 | 0.45 | 63.38 | 307.73 | 6.62 | 2.50 | 0.17 | 22.25 | 1.47 | 67.50 |
| | SD | 0.63 | 10.23 | 0.03 | 2.82 | 5.19 | 0.60 | 0.58 | 0.05 | 3.10 | 0.20 | 2.89 |
| M-HD | Mean | 7.42 | 148.75 | 0.49 | 66.24 | 303.57 | 6.79 | 2.00 | 0.14 | 17.50 | 1.19 | 73.00 |
| | SD | 0.45 | 1.50 | 0.00 | 3.88 | 3.06 | 0.90 | 0.82 | 0.06 | 0.58 | 0.18 | 2.16 |

TAMAPAL1

| | | | | | | | | | | | | |
|---|---|---|---|---|---|---|---|---|---|---|---|---|
| F-C | Mean | 7.56 | 151.25 | 0.49 | 64.87 | 308.81 | 11.71 | 2.00 | 0.23 | 20.00 | 2.22 | 71.00 |
| | SD | 0.08 | 3.86 | 0.02 | 2.60 | 6.21 | 4.33 | 0.00 | 0.09 | 4.69 | 0.56 | 5.29 |
| F-LD | Mean | 7.23 | 141.00 | 0.46 | 63.02 | 310.48 | 6.74 | 2.00 | 0.15 | 18.50 | 1.28 | 72.50 |
| | SD | 0.38 | 5.03 | 0.02 | 2.80 | 18.49 | 2.10 | 0.82 | 0.10 | 5.32 | 0.58 | 4.20 |
| F-HD | Mean | 7.5 | 141.25 | 0.45 | 59.92 | 316.65 | 5.91 | 2.00 | 0.12 | 18.25 | 1.07 | 72.50 |
| | SD | 0.54 | 11.44 | 0.03 | 6.78 | 34.68 | 0.70 | 0.00 | 0.01 | 0.96 | 0.09 | 2.08 |
| M-C | Mean | 7.54 | 146.25 | 0.48 | 64.19 | 303.31 | 9.98 | 2.00 | 0.20 | 23.00 | 2.28 | 67.00 |
| | SD | 0.64 | 8.18 | 0.03 | 4.21 | 4.32 | 2.35 | 0.82 | 0.08 | 7.26 | 0.81 | 4.69 |
| M-LD | Mean | 7.58 | 151.25 | 0.49 | 64.43 | 310.21 | 9.19 | 2.00 | 0.18 | 25.75 | 2.36 | 66.00 |
| | SD | 0.40 | 7.23 | 0.01 | 2.71 | 10.72 | 0.67 | 0.00 | 0.01 | 6.85 | 0.67 | 7.62 |
| M-HD | Mean | 7.20 | 142.50 | 0.46 | 64.29 | 307.93 | 8.62 | 2.25 | 0.18 | 24.00 | 2.05 | 67.00 |
| | SD | 1.03 | 20.57 | 0.06 | 3.54 | 5.48 | 3.47 | 0.50 | 0.05 | 3.16 | 0.82 | 4.24 |

### RetroMAD1

All data for low and high dose males were unremarkable and comparable against the controls. The females exhibited higher percentages of WBC (White Blood Cells) although the mean numbers were within the standard deviation of the controls. Thrombocyte counts were seen to increase by 40% over the control and there was no significant difference between the low and high doses which will indicate a risk of abnormal blot clots if these data are repeated in primate toxicology trials. Also, it should be noted that these values were not significantly elevated if compared to the other mice from the other control groups for RetroGAD1 and Tamapal1.

In the males, all parameters were within the standard deviation of the mean indicating sex-related metabolic differences may account for the observations in the female group.

### RetroGAD1

In the female population, both low and high doses resulted in a large drop in numbers of WBC, B Neutrophil, S Neutrophil and Lymphocytes. In the male populations, only WBC dropped in the low dose but not in the high dose and S Neutrophils only in the high dose. More parameter deviations were observed in females compared to males.

### Tamapal1

In the female population, only high doses of Tamapal1 consistently caused a drop in WBC, B Neutrophils, S Neutrophils and Lymphocytes in a dose dependent manner. In the males, all parameters were within the standard deviation of the mean indicating sex-related metabolic differences may account for the observations noted only in the female group.

All the drugs tested showed that hematology parameters were very much more affected in female compared to male populations probably due to sex-related metabolic differences. Males were generally unaffected. Nonetheless, as all rats survived and behaved normally, histology data would have to be reviewed in order to get a clearer picture. Nevertheless, it shows that the rats survived 100× and 500× the therapeutic dose given to cats and dogs in multicentre trials for experimentally treating FIV, FeLV and CPV2. The data also shows that RetroGAD1 gave more parameter deviations in females even though the protein concentrations given were the lowest of the three indicating that drug safety from a hematology safety viewpoint was as follows—RetroMAD1 >Tamapal1 >RetroGAD1.

### REFERENCES

1. Bergmeyer H U, (1980); Clin. Chimica. Acta., 105: 147-154;

2. Brudno M., (2003b); Bioinformatics; 19 Suppl 1:154-162;

3. Cole et. al. (2002); PNAS, V99(4):1813-1818;

4. Karlin and Altschul (Karlin & Altschul (1993) Proc. Natl. Acad. Sci. USA 90: 5873-5877)

5. Sambrook and Green, Molecular Cloning: A Laboratory Manual (Fourth Edition), Cold Springs Harbor Laboratory, New York (2012);

6. Tietz N W, Rinker A D, Shaw L M (1983); J. Clin. Chem. Clin. Biochem., 21:731-748;

7. Wang et. al. (2003); J. Immunol. 170:4708-4716,

8. U.S. Pat. No. 6,652,861

9. Hoskin D. W. and Ramamoorthy A. (2008) Biochemica et Biophysica Acta 1778:357-375;

10. Tang Y Q, Yuan J, Osapay G et al. (October 1999). “A cyclic antimicrobial peptide produced in primate leukocytes by the ligation of two truncated alpha-defensins”. Science 286 (5439): 498-502;

11. Leonova L, Kokryakov V N, Aleshina G et al. (September 2001). “Circular minidefensins and posttranslational generation of molecular diversity”. J. Leukoc. Biol. 70 (3): 461-4.

12. Wang Wet at Activity of alpha- and theta-defensins against primary isolates of HIV-1. Journal of Immunology 173(1): 515-520 (2004);

13. U.S. Pat. No. 6,652,861;

14. Tang C et at 2010 Org Biomol Che 8:984-7;

15. Fan, J-M., et al, Mol Biotechnol, 2008, 39, 79-86;

16. Wang et al, Cell, 1999, 99, 433-442;

17. Di Francesco, C. (2007) FDA Approves Nexavar for Patients with Inoperable Liver Cancer: FDA News Release. U.S. Food and Drug Administration: Protecting and Promoting Your Health. <http://www.fda.gov/NewsEvents/Newsroom/PressAnnouncements/2007/ucm109030.htm>Date accessed: 12 Apr. 2013.

18. Gedaly R, Angulo P, Hundley J, Daily M F, Chen C, Koch A and Evers B M (2010) PI-103 and Sorafenib Inhibit Hepatocellular Carcinoma Cell Proliferation by Blocking Ras/Raf/MAPK and PI3K/AKT/mTOR Pathways. Anticancer Research 30: 4951-4958.

19. Rohana, Y., Clum, S., Wetzel, M., Krishnamurthy, H. M., Padmanabhan, R. (2000) Purified NS2B/NS3 Serine Protease of Dengue Virus Type 2 Exhibits Cofactor NS2B Dependance for Cleavage of Substrates with Dibasic Amino Acids in Vitro. The Journal of Biological Chemistry 275: 9963-9969.

20. Malich et al., (1997) The sensitivity and specificity of the tetrazolium assay for detecting the in vitro cytotoxicity of 20 chemicals using human cell lines. Toxicology 124 (3): 179-92.

21. Mayo L D and Donner D B (September 2001). “A phosphatidylinositol 3-kinase/Akt pathway promotes translocation of Mdm2 from the cytoplasm to the nucleus”. Proc Natl Sci USA. 98 (20): 11598-603

22. Hashimoto, S. and S. Imaoka (2013). “Protein-disulfide isomerase regulates the thyroid hormone receptor-mediated gene expression via redox factor-1 through thiol reduction-oxidation.” J Biol Chem 288(3): 1706-1716.

23. Nirde, P., et al. (2010). “Heat shock cognate 70 protein secretion as a new growth arrest signal for cancer cells.” Oncogene 29 (1): 117-127.

24. Sliskovic, I. and B. Mutus (2006). “Reversible inhibition of caspase-3 activity by iron(III): potential role in physiological control of apoptosis.” FEBS Lett 580 (9): 2233-2237.

25. Tai, C. J., et al. (2012). “Survivin-mediated cancer cell migration through GRP78 and epithelial-mesenchymal transition (EMT) marker expression in Mahlavu cells.” Ann Surg Oncol 19 (1): 336-343.

26. Wu, S. and H. Le (2013). “Dual roles of PKM2 in cancer metabolism.” Acta Biochim Biophys Sin (Shanghai) 45(1): 27-35.

27. Wei, P. L., et al. (2012). “Silencing of glucose-regulated protein 78 (GRP78) enhances cell migration through the upregulation of vimentin in hepatocellular carcinoma cells.” Ann Surg Oncol 19 Suppl 3: S572-579.

28. Tietz N W, Rinker A D, Shaw L M (1983). IFCC methods for the measurement of catalytic concentration of enzymes. Part 5. IFCC method for alkaline phosphatase. J. Clinical. Chem. Clinical. Biochem., 21: 731-748.

1-85. (canceled)

86. A method of treating cancer in a subject, the method comprising a step of administering at least one fusion protein comprising at least one polypeptide B, which is a Type 1 Ribosome Inactivating Protein (RIP) or fragment thereof; and
(i) at least one polypeptide A which is an antimicrobial peptide; and/or
(ii) at least one polypeptide C which is a Cationic Antimicrobial Peptide (CAP) or fragment thereof
to the subject in need thereof.

87. The method according to claim 86, wherein the fusion protein regulates the major histocompatibility (MHC) class I pathway.

88. The method according to claim 86, wherein the fusion protein further comprises at least one polypeptide D, which is a synthetic anticancer polypeptide, or a fragment thereof.

89. The method according to claim 88, wherein the polypeptide D is a Bax-derived membrane-active peptide.

90. The method according to claim 88, wherein the polypeptide D is selected from the group consisting of (KLAKLAK)2, SSX2 and D-K4R2L9 .

91. The method according to claim 86, wherein the polypeptide A is a defensin.

92. The method according to claim 91, wherein the defensin is selected from the group consisting of an alpha defensin, beta defensin, gamma defensin, and big defensin an analogue, or a fragment thereof.

93. The method according to claim 86, wherein the fusion protein comprises the structure A-B-C, A-C-B, C-A-B, C-B-A, B-A-C, B-C-A, A-B-C-C, A-B, B-C, B-C-C, C-C-B-C-C, C-B-C, C-B-D, C-D-B, B-D-C, B-C-D, D-C-B or D-B-C.

94. The method according to claim 86, wherein the fusion protein comprises polypeptides A, B and C.

95. The method according to claim 86, further comprising at least one linker peptide between each of the polypeptides.

96. The method according to claim 95, wherein the linker peptide has SEQ ID NO: 3 or SEQ ID NO: 27.

97. The method according to claim 86, wherein polypeptide A is:
(i) a theta defensin selected from the group consisting of Rhesus minidefensin (RTD-1), RTD-2, RTD-3, Retrocyclin-1, Retrocyclin-2, Retrocyclin-3, synthetic retrocyclin congener RC100, RC101, RC102, RC103, RC104, RC105, RC106, RC107, RC108, RC110, RC111, RC112, RC113 and RC114;
(ii) an alpha-defensin selected from the group consisting of human neutrophil protein 1 (HNP-1), HNP-2, HNP-3, HNP-4, Human defensin 5 and Human defensin 6, an analogue, or a fragment thereof; or
(iii) a beta-defensin selected from the group consisting of DEFB 1, DEFB 4A, DEFB 4B, DEFB 103A, DEFB 103B, DEFB 104A, DEFB 104B, DEFB 105A, DEFB 105B, DEFB 106A, DEFB 106B, DEFB 107A, DEFB 107B, DEFB 108B, DEFB108 P1-4, DEFB 109 P1, DEFB 109 P1B, DEFB 109 P2-3, DEFB 110, DEFB 112-119 and DEFB 121-136.

98. The method according to claim 86, wherein polypeptide B is selected from the group consisting of α-Ebulitin, β-Ebulitin, γ-Ebulitin, Nigritin f1, Nigritin f2, Amarandin-S, Amaranthus antiviral/RIP, Amarandin-1, Amarandin-2, Amaranthin, Atriplex patens RIP, Beta vulgaris RIP, Betavulgin, Celosia cristata RIP, Chenopodium album RIP, CAP30B, Spinaca oleracea RIP, Quinqueginsin, Asparin 1, Asparin 2, Agrostin, Dianthin 29, DAP-30, DAP-32, Dianthin 30, Dianthus chinensis RIP1, Dianthus chinensis RIP2, Dianthus chinensis RIPS, Lychnin, Petroglaucin, Petrograndin, Saponaria ocymoides RIP, Vacuolas saporin, Saporin-1, Saporin-2, Saporin-3, Saporin-5, Saporin-6, Saporin-7, Saporin-9, Vaccaria hispanica RIP, Benincasin, Alpha-benincasin, Beta-benincasin, Hispin, Byrodin I, Byrodin II, Colocin I, Colocin 2, Cucumis figarei RIP, Melonin, C. moschata RIP, Cucurmosin, Moschatin, Moschatin I, Moschatin II, Moschatin III, Moschatin IV, Moschatin V, Pepocin, Gynostemmin I, Gynostemmin II, Gynostemmin III, Gynostemmin IV, Gynostemmin V, Gynostemma pentaphyllum RIP, Lagenin, Luffaculin, Luffangulin, Luffin-alpha, Luffin-B, MOR-I, MOR-II, Momordin II, Alpha-momorcharin, Beta-momorcharin, Delta-momorcharin, Gamma-momorcharin, Momorcochin, Momorcochin-S, Sechiumin, Momorgrosvin, Trichoanguin, Alpha-kirilowin, Beta-kirilowin, Alpha-trichosanthin, TAP-29, Trichokirin, Trichomislin, Trichosanthin, Karasurin-A, Karasurin-B, Trichomaglin, Trichobakin, Crotin 2, Crotin 3, Euserratin 1, Euserratin 2, Antiviral Protein GAP-31, Gelonin, Hura crepitans RIP, Curcin, Jathropa curcas RIP, Mapalmin, Manutin 1, Manutin 2, Alpha-pisavin, Charibdin, Hyacinthus orientalis RIP, Musarmin 1, Musarmin 2, Musarmin 3, Musarmin 4, Iris hollandica RIP, Cleroendrum aculeatum RIP, CIP-29, CIP-34, Crip-31, Bouganin, Bougainvilla spectbilis RIP, Bougainvillea×buttiana Antiviral protein 1 (BBAP1), malic enzyme 1 (MED, ME2, MAP-S, pokewood antiviral protein (PAPa-1), PAPa-2, PAP-alpha, PAP-I, PAP-II, PAP-S, PD-SI, DP-S2, Dodecandrin, Anti-viral protein PAP, PIP, PIP2, Phytolacca octandra anti-viral protein, Phytolacca, octandra anti-viral protein II, Hordeum vulgare RIP-I, Hordeum vulgare RIP-II, Hordeum vulgare sub sp. Vulgare Translational inhibitor II, Secale cereale RIP, Tritin, Zea, diploperemis RIP-I, Zea diploperemis RIP-II, Malus×domestica RIP, Momordica Anti-HIV Protein (MAP30), Gelonium multiflorum (GAP31), pokewood antiviral protein (PAP), Mirabilis expansa 1 (MED, malic enzyme 2 (ME2), Bougainvillea×buttiana antiviral protein 1 (BBAP1), phage MU1, betavulgin (Bvg), curcin 2, saporin 6, Dianthin 29, Maize ribosome-inactivating protein (B-32), Ribosome-Inactivating Protein from Tobacco (TRIP), beetin (BE), BE27, Mirabilis antiviral protein (MAP), Trichosanthin (TCS), alpha-luffin, DAP30, DAP32, alpha-Momorcharin (α-MMC), β-MMC luffin and saporin.

99. The method according to claim 86, wherein polypeptide C is selected from the group consisting of Aurein 1.2, Antiviral protein Y3, Alloferon 1, Lactoferricin B, Siamycin II, Cecropin A(1-8)-Magainin 2(1-12) hybrid (CE-MA), Maximin 1, Maximin 3, Maximin 4, Maximin 5, Brevinin-1, Ranatuerin-2P, Cecropin A, Melittin, Indolicidin, Dermaseptin 1, Dermaseptin 4, Dermaseptin-S1, and Latarcin, Siamycin I and II, Mundticin KS, Enterocin CRL-35, NP-06, Plectasin, Circulin A, Circulin B, Ginkbilobin, Alpha-Basrubin, Lunatusin, Sesquin, Kalata B1, Kalata B8, Cycloviolin A, Cycloviolin B, Cycloviolin C, Cycloviolin D, Vary Peptide E, Palicourein, VHL-1, Circulin C, Circulin D, Circulin E, Circulin F, Cycloviolacin O13, Cycloviolacin O14, Cycloviolacin O24, Cycloviolacin Y1, Cycloviolacin Y4, Cycloviolacin Y5, Polyphemusin I, Tachyplesin I, Rat Defensin NP3, Rat Defensin NP4, Cow cathelicidin BMAP-28, Human α-Defensin HNP-1, Human α-Defensin HNP-2, Human α-Defensin HNP-3, Human α-Defensin HNP-4, Human α-Defensin HNP-5, Human α-Defensin HNP-6, Human β-defensin III HBD3, LL-37 Cathelicidin Human/Chimpanzee, Caerin 1.1, Uperin 3.6, Ranatuerin 6, Esculentin-1, Gaegurin 5 and Gaegurin 6.

100. The method according to claim 86, wherein;
(i) the Type 1 RIP is MAP30, the CAP is Dermaseptin 1 and the polypeptide A is Retrocyclin 101
(ii) the Type 1 RIP is MAP30, the CAP is Alloferon 1 and the polypeptide A is Tachyplesin; or (iii) the Type 1 RIP is MAP30, the polypeptide D is (KLAKKLAK)2 and the polypeptide A is Gaegurin 5.

101. The method according to claim 100, wherein:
the fusion protein in (i) comprises the amino acid sequence SEQ ID NO: 1;
the fusion protein in (ii) comprises the amino acid sequence SEQ ID NO: 34; and
the fusion protein in (iii) comprises the amino acid sequence SEQ ID NO: 35.

102. The method according to claim 86, wherein the fusion protein is capable of PI3 kinase inhibition.

103. The method according to claim 86, wherein the fusion protein is suitable for oral administration.

104. The method according to claim 86, wherein the fusion protein further comprises at least one G-rich oligonucleotide or siRNA.

105. The method according to claim 86, wherein the cancer is selected from the group consisting of Non-Hodgkin's Lymphoma, brain, lung, colon, epidermoid, squamous cell, bladder, gastric, pancreatic, breast, head, neck, renal, kidney, liver, ovarian, prostate, colorectal, uterine, rectal, oesophageal, testicular, gynecological, thyroid cancer, melanoma, hematologic malignancies such as acute myelogenous leukemia, multiple myeloma, chronic myelogneous leukemia, myeloid cell leukemia, glioma, pontine glioblastoma, Kaposi's sarcoma, or any other type of solid or liquid cancer.

106. The method according to claim 105, wherein the cancer is liver cancer.

107. The method according to claim 87, wherein the regulating of the MHC class I pathway is by the upregulation of at least one MHC class 1 antigen presentation molecule selected from the group consisting of sequestosome-1, calnexin, heat shock cognate, calreticulin, endoplasmin and protein disulfide-isomerase.
